(19) United States
(12) Patent Application Publication
(10) Pub. No.: US 2023/0028445 A1
(43) Pub. Date: Jan. 26, 2023

(54) IDENTIFICATION OF GENOMIC STRUCTURAL VARIANTS USING LONG-READ SEQUENCING

(71) Applicant: BLACK HAWK GENOMICS, LLC, Lubhock, TX (US)

(72) Inventors: Tatum Owatha, Lubock, TX (US); Ji Lee, Sparks, MD (US)

(21) Appl. No.: 17/774,345

(22) PCT Filed: Oct. 12, 2020
(86) PCT No.: PCT/US2020/055293

§ 371 Date: May 4, 2022

Related U.S. Application Data
(60) Provisional application No. 62/981,146, filed on Feb. 25, 2020.
(60) Provisional application No. 62/913,886, filed on Oct. 11, 2019.

(51) Int. Cl. C12Q 1/6883; C12Q 1/6811

(52) U.S. Cl. CPC C12Q 1/6883; C12Q 1/6811; C12Q 1/6806

(57) ABSTRACT

Provided herein are systems and methods for detecting genomic structural variants using a non-application gene-editing sample preparation followed by long-read sequencing.

### CROSS-REFERENCE TO RELATED APPLICATIONS

This application claims the benefit of U.S. Provisional Application No. 62/913,886 filed Oct. 11, 2019; and of U.S. Provisional Application No. 62/981,146, filed Feb. 25, 2020, each of which is incorporated herein by reference.

### BACKGROUND OF THE INVENTION

A genetic abnormality or genomic variation in the genetic makeup of an individual can cause a genetic disease or disorder in the individual. The genetic abnormality or genomic variation can range for a discrete mutation in a single base (e.g. single nucleotide variant) to a chromosomal abnormality or structural variant (SV) (e.g. copy number variant, segmental inversions, etc.) comprising the rearrangement, addition or deletion of one or more genes. Currently, more than 100,000 genetic variants have been classified as disease-causing in public databases. For example, sickle cell disease is caused by a single nucleotide mutation in the beta-globin gene; Fragile X syndrome is caused by tandem duplication of the CGG trinucleotide repeated over 200 times; and Down Syndrome is commonly caused by complete duplication of chromosome 21. Short-read sequencing technologies can identify small genomic variations such as single nucleotide variants, insertions and deletions, with high accuracy. However, these technologies are unable to identify structural variants larger than a few hundred base pairs with good accuracy. Several methods have emerged to try to detect structural variants; but they all have their limitations. For example, microscopy using fluorescent probes is low-throughput, is quite expensive, and has low resolution. Quantitative PCR (qPCR) and microarray assays are high-throughput and inexpensive but cannot identify unknown structural variants. Short-read sequencers, which are high-throughput and inexpensive, have difficulty resolving SVs and frequently are coupled with another technology, such as optical mapping or linked read sequencing, to identify SVs accurately. Whole genome sequencing using long-read sequencers can be used to detect large structural variants; however, whole genome sequencing is expensive, and some long-read sequencers have difficulty resolving very large structural variants. As such, there is an immediate need, especially in the clinical setting, for a fast, high-throughput yet cost-effective method to identify genomic structural variants, in particular, de novo structural variants.

### SUMMARY OF THE INVENTION

In one aspect, provided herein is a method for identifying a set of guide RNAs (gRNAs) that are hybridizable to a genomic region of interest in a genome comprising designing a plurality of gRNAs, wherein at least one gRNA is hybridizable to a target site within the genomic region of interest and is configured to produce a genomic variant that comprises at least 1000 bp; and said plurality of gRNAs comprises a plurality of CRISPR RNAs (crRNAs), wherein said plurality of crRNAs comprises a GC of at least about 20% to about 80%.

In another aspect, provided herein is a method for identifying a set of guide RNAs (gRNAs) that are hybridizable to a genomic region of interest in a genome comprising designing a plurality of gRNAs, wherein at least one gRNA is hybridizable to a target site within the genomic region of interest and is configured to produce a genomic variant that comprises at least 1000 bp; and said plurality of gRNAs comprises a plurality of CRISPR RNAs (crRNAs), wherein said plurality of crRNAs comprises a self-complementarity score of zero.

In another aspect, provided herein is a method for identifying a set of guide RNAs (gRNAs) that are hybridizable to a genomic region of interest in a genome comprising designing a plurality of gRNAs, wherein at least one gRNA is hybridizable to a target site within the genomic region of interest and is configured to produce a genomic variant that comprises at least 1000 bp; and said plurality of gRNAs comprises a plurality of CRISPR RNAs (crRNAs), wherein said plurality of crRNAs comprises an efficiency score of about 0.2.

In another aspect, provided herein is a method for identifying a set of guide RNAs (gRNAs) that are hybridizable to a genomic region of interest in a genome comprising designing a plurality of gRNAs, wherein at least one gRNA is hybridizable to a target site within the genomic region of interest and is configured to produce a genomic variant that comprises at least 1000 bp; and said plurality of gRNAs comprises a plurality of CRISPR RNAs (crRNAs), wherein said plurality of crRNAs comprises a mismatch profile of MM0<2, MM1<3, MM2<3, and MM3<21.

In some embodiments, the plurality of crRNAs comprises a mismatch profile of MM3<5.

In another aspect, provided herein is a method of detecting a genomic variant in a sample, the method comprising enriching said sample for a genomic region of interest comprising said genomic variant using a gene-editing based approach; and sequencing said enriched sample comprising said genomic region of interest using long-read sequencing.

In some embodiments, said genomic variant comprises a structural variant. In some cases, said genomic variant comprises at least 50 bp. In some embodiments, said genomic variant comprises a structural variant. In some cases, said genomic variant comprises at least 1000 bp.

In some embodiments, said gene-editing based approach comprises use of a clustered regularly interspersed short palindromic repeats (CRISPR)-Cas system. In some cases, said CRISPR-Cas system comprises Cas9.

In some embodiments, step (a) of enriching of said sample further comprises amplification of said genomic region of interest. In some embodiments, step (a) of enriching said sample does not require amplification of said genomic region of interest. In some embodiments, step (a) of enriching of said sample further comprises coupling a sequence of dAMPs to said genomic variant. In some embodiments, step (a) of enriching of said sample further comprises coupling a plurality of barcode molecules to said genomic variant. In some embodiments, step (a) of enriching of said sample further comprises coupling said genomic variant to a magnetic bead.

In some embodiments, said long-read sequencing comprises nanopore sequencing. In some embodiments, said long-read sequencing comprises single molecule, real-time (SMRT) sequencing.

In some embodiments, said CRISPR-Cas system further comprises a crRNA comprising a sequence of Tables 1-117.

In some embodiments, said genomic region of interest comprises two or more repeat regions. In some embodiments, said genomic region of interest comprises a GC content of greater than 30%.

In some embodiments, said sample comprises at least 10 genomic regions of interest.

In some embodiments, said genomic variant is associated with a disorder. In some cases, the disorder is selected from the group consisting of acute lymphoblastic leukemia (ALL), alpha-thalassemia, ataxia-telangiectasia (AT), autosomal recessive deafness 16, autosomal recessive deafness 22, beta-thalassemia, breast cancer, Canavan disease, cancer, celiac disease, chronic myeloid leukemia (CIVIL), cystic fibrosis, cystinosis, deafness infertility syndrome (DIS), Duchenne muscular dystrophy, Ehlers-Danlos syndrome type III and IV, Ellis-van Creveld syndrome, Fabry disease, familial adenomatous polyposis (FAP), familiar cutaneous melanoma, Fragile X, gastric cancer (including hereditary diffuse gastric cancer), Gaucher disease, hereditary predisposition to develop cancer, Huntington disease, hypophosphatasia (HPP), incontinentia pigmenti, Krabbe disease, Leber congenital amaurosis (LCA), Loeys-Dietz syndrome, Long QT syndrome, Lynch syndrome, Marfan syndrome, mental disorder, medium-chain acyl-coenzyme A dehydrogenase (MCAD) deficiency, MUTYH-associated polyposis, neuroblastoma, neuronal ceroid-lipofuscinoses (NCLs), Niemann-Pick Type C disease, pancreatic cancer syndromes, papillary renal carcinoma, Parkinson disease, phenylketonuria, Pompe disease, propiopnic acidemia, rheumatoid arthritis, solid tumors, spinal muscular atrophy, spinocerebellar ataxia, susceptibility to breast cancer, Tay-Sachs disease, very long-chain acyl-coenzyme A dehydrogenase deficiency, Von Hippel-Lindau syndrome, Wilms tumor, Wilson disease, Wolfram syndrome type 1, X-linked creatine deficiency syndrome, X-linked hemophilia A, X-linked retinitis pigmentosa.

Provided herein is a method of designing a probe to target a genomic region of interest, the method comprising designing a plurality of nucleic acid probe options to target said genomic region of interest; selecting a first set of candidates from said plurality of nucleic acid probe options with a GC content of at least 20%; selecting a second set of candidates from said first set of candidates with a self-complementarity score of zero or a complementarity score of 1; selecting a third set of candidates from said second set of candidates with an efficiency greater than 0.2; and selecting a fourth set of candidates from said third set of candidates with a mismatch profile of MM0=0 or MM0=1, MM1=0 or MM1=1 or MM1=2, MM2=0 or MM2=1 or MM2=2, and MM3<21, wherein said fourth set of candidates comprises said probe to target a genomic region of interest, wherein said fourth set of candidates comprises said probe to target a genomic region of interest.

In some embodiments, the fourth set of candidates comprises a mismatch profile of MM3<5. In some embodiments, said designing comprises using CHOPCHOP.

In some embodiments, said first set of candidates have a GC content of about 40% to about 80%.

In some embodiments, said nucleic acid probe of interest comprises a crRNA. In some embodiments, the probability of said crRNA cutting said genomic region of interest is greater than or equal to 80%. In some embodiments, the method further comprises estimating on-target value of said crRNA. In some embodiments, the method further comprises estimating off-target value of said crRNA.

In another aspect, provided herein is a kit comprising a set of guide RNAs (gRNAs) that are hybridizable to a genomic region of interest in a genome comprising designing a plurality of gRNAs, wherein at least one gRNA is hybridizable to a target site within the genomic region of interest and is configured to produce a genomic variant that comprises at least 1000 bp; and said plurality of gRNAs comprises a plurality of CRISPR RNAs (crRNAs), wherein said plurality of crRNAs comprises a GC of at least about 40% to about 80%.

In another aspect, provided herein is a kit comprising a set of guide RNAs (gRNAs) that are hybridizable to a genomic region of interest in a genome comprising designing a plurality of gRNAs, wherein at least one gRNA is hybridizable to a target site within the genomic region of interest and is configured to produce a genomic variant that comprises at least 1000 bp; and said plurality of gRNAs comprises a plurality of CRISPR RNAs (crRNAs), wherein said plurality of crRNAs comprises a self-complementarity score of zero.

In another aspect, provided herein is a kit comprising a set of guide RNAs (gRNAs) that are hybridizable to a genomic region of interest in a genome comprising designing a plurality of gRNAs, wherein at least one gRNA is hybridizable to a target site within the genomic region of interest and is configured to produce a genomic variant that comprises at least 1000 bp; and said plurality of gRNAs comprises a plurality of CRISPR RNAs (crRNAs), wherein said plurality of crRNAs comprises an efficiency score of about 0.2.

In another aspect, provided herein is a kit comprising a set of guide RNAs (gRNAs) that are hybridizable to a genomic region of interest in a genome comprising designing a plurality of gRNAs, wherein at least one gRNA is hybridizable to a target site within the genomic region of interest and is configured to produce a genomic variant that comprises at least 1000 bp; and said plurality of gRNAs comprises a plurality of CRISPR RNAs (crRNAs), wherein said plurality of crRNAs comprises a mismatch profile of MM0=0 or MM0=1, MM1=0 or MM1=1 or MM1=2, MM2=0 or MM2=1 or MM2=2, and MM3<21.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 provides exemplary genomic abnormalities and variants.

FIG. 2 provides an exemplary target enrichment sample preparation approach, in accordance with the embodiments provided herein.

FIG. 3 provides an exemplary design approach for crRNA probes, in accordance with the embodiments provided herein.

FIGS. 4A and 4B provide exemplary coverage of a crRNA probe embodiment, in accordance with the embodiments provided herein.

FIG. 5 provides an exemplary computer control system that is programmed to implement the methods provided, in accordance with the embodiments provided herein.

FIG. 6 provides an exemplary design approach for crRNA probes, in accordance with the embodiments provided herein.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise.

The term “subject,” as used herein, generally refers to an animal, such as a mammal (e.g., human) or avian (e.g., bird), or other organism, such as plant. For example, the subject can be a vertebrate, a mammal, a rodent (e.g., a mouse), a primate, a simian, or a human. Animals may include, but are not limited to, farm animals, sport animals, and pets. A subject may be a healthy or asymptomatic individual, an individual that has or is suspected of having a disease (e.g., a genetic disorder) or a pre-disposition to a disease, and/or an individual that is in need of therapy or suspected of needing therapy. A subject can be a patient.

The term “genome,” as used herein, generally refers to genomic information from a subject, which may be, for example, at least a portion or an entirety of a subject's hereditary information. A genome can be encoded either in DNA or in RNA. A genome can include the sequence of all chromosomes together in an organism. For example, the human genome ordinarily has a total of 46 chromosomes. The sequence of all these together may constitute a human genome.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, sequencing system by Illumina®, Pacific Biosciences (PacBio®), Oxford Nanopore®, Life Technologies (Ion Torrent®), Roche®, Genapsys®, and MGI Tech®. Sequencing may be performed without using nucleic acid amplification. Alternatively, or in addition, sequencing may be performed using nucleic acid amplification, polymerase chain reaction (PCR) (e.g. digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample may include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, of fine needle aspirate. The sample may be a fluid sample, such as blood sample, urine sample, or saliva sample. The sample may be a skin sample. The sample may be a cheek swab. The sample may be a plasma or serum sample. The sample may include cells or may be cell-free. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “short read,” as used herein, generally refers to a read length of a DNA or RNA polynucleotide of about 100 to about 600 bp.

The term “long read,” as used herein, generally refers to a read length of a DNA or RNA polynucleotide of greater than 1 Kbp.

The term “ribonucleoprotein (RNP),” as used herein is a ribonucleoprotein is a ribonucleic acid (RNA)-protein complex.

The term “CRISPR-Cas system,” as used herein, generally refers the clustered regularly short palindromic repeats (CRISPR system) which comprises an array of two types of DNA sequences: (i) repetitive, flanking DNA sequences; and (ii) spacer sequences that are endogenously derived from a virus, and can be used to target DNA or RNA sequences for cleaving using the CRISPR-associated (Cas) enzyme (ribonucleoprotein) complex that are used to cleave the CRISPR sites that are complementary to those in spacer regions.

The term “barcoding,” as used herein, is the ligation of known, unique sequences to target DNA molecules, between the adapter and the ROI in order for the target sequence recognition in the downstream analysis, i.e. post-base calling.

The term “multiplexing,” as used herein, is the running of multiple samples in a single flow cell, identifying each sample's DNA molecules through unique ‘barcode’ molecules that have been attached to the DNA ends. The decoded sequences of a sample's DNA will be identified downstream once the sequences have been basecalled.

The term “crRNA,” as used herein, are the RNA sequences that recognize the target site. Together with the tracrRNA, this forms a single guide RNA (sgRNA) and when several are used together, gRNA.

The term “tracrRNA,” as used herein, refers to trans-activating-crRNA specific to Type II Cas/CRISPR system. It is used to process the pre-crRNA along with an RNase III. The tracrRNA provides structural support to the ribonucleoprotein and anneals to the pre-crRNA for processing via the internal endonuclease activity of the Cas protein. Non-limiting examples of Cas enzymes can include Cas1, Cas1B, Cas2, Cas3, Cas4, Cas5, Cash, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof. In some cases, a catalytically dead Cas protein can be used, for example a dCas9. An unmodified CRISPR enzyme can have DNA cleavage activity, such as Cas9.

The term “protospacer,” as used herein, refers to a sequence acquired from a pathogenic organism's DNA molecule. The sequence is converted into DNA and forms the gene of the crRNA, which, along with the PAM in the substrate sequence, directs the Cas-crRNA-tracrRNA ternary complex to cleave target molecule.

The term “protospacer adjacent motif (PAM),” as used herein, refers to pathogenic sequences from host sequences i.e. the crRNA gene. It is adjacent to the 3′ end of the protospacer and facilitates the pathogen's sequence to be cut by the Cas-crRNA-tracrRNA ternary complex. In Type II CRISPR systems, NGG (where N is any nucleotide as per FASTA conventions), defines the sequences that will be cleaved. If a PAM is 3′ of crRNA sequence in the DNA, the crRNA-tracrRNA-Cas9 ternary complex will not cleave the host DNA/genome sequence.

The term “untranslated region (UTR),” as used herein, refers to the untranslated region (UTR) of a mRNA transcript and is present both at the 5′ and 3′ ends of the protein coding region. It is not translated via the protein synthesis process by the ribosome.

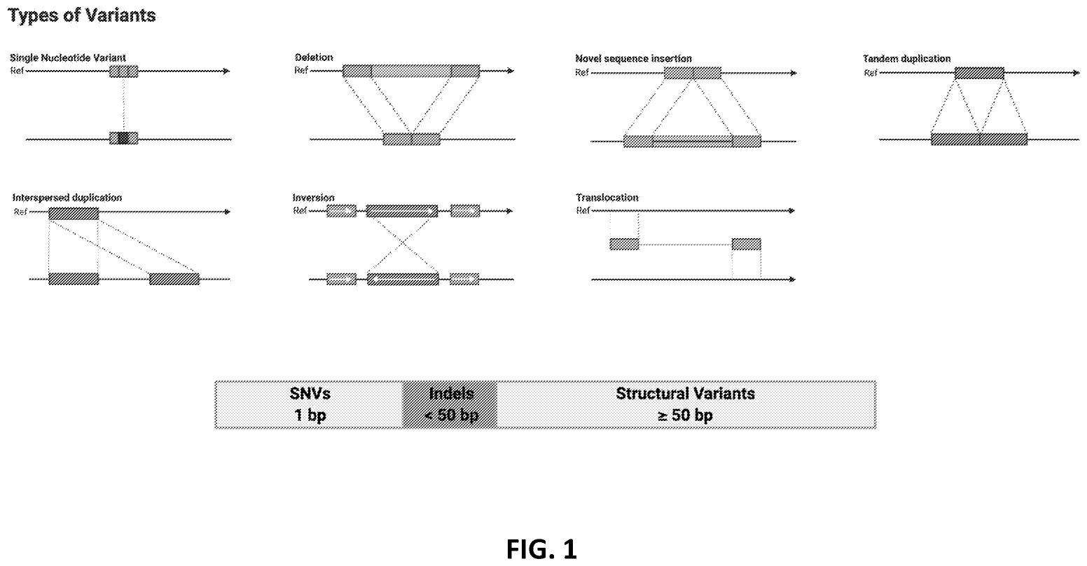

The human genome has many different types of genomic variations that range in size and type. FIG. 1 shows examples of genomic variations. A single nucleotide variant is a substitution of a single nucleotide at a specific position in the genome. A deletion is a loss of one or more nucleotides in the genome, ranging from a single base to an entire chromosome. In contrast, an insertion is the addition of one or more nucleotides to the genome. A tandem repeat consists of two or more adjacent copies of a sequence of at least two nucleotides in length. A tandem duplication occurs when a nucleotide sequence, which itself can contain a repeated sequence, is copied into two adjacent copies. Interspersed duplication differs from tandem duplication or repeat in that the repeated sequence is dispersed throughout the genome and is nonadjacent to the original copy. Inversion is a chromosome rearrangement in which a segment of a gene, structural element or chromosome is reversed end to end. Translocation is the unusual rearrangement of chromosomes. Copy number variants is a type of structural repetition in which one or more parts of the genome are repeated.

The types of genomic variants can be categorized based on the number of nucleotides involved. Single nucleotide variants (SNVs) affect a single nucleotide or base pair. Small insertions and deletions, commonly called indels, are shorter than 50 nucleotides in length. Structural variants are changes in the structure of chromosome and generally affect 50 or more nucleotides. The typical human genome has about 8 million bases that differ from a reference due to SNVs and indels. The typical human genome has about 20,000 structural variants that differ from the reference and affects about 10 million bases.

In one aspect, provided herein are systems and methods to detect one or more genomic variants in a sample, comprising (a) preparing a sample for sequencing using a non-amplification-based, gene-editing based approach, and (b) long-read sequencing, as described herein elsewhere.

In another aspect, provided herein are systems and methods for conducting a diagnostic assay for a genetic disorder, comprising (a) preparing a sample for sequencing using a non-amplification-based, gene-editing based approach, and (b) long-read sequencing, as described herein elsewhere.

In some cases, the one or more genomic variants comprise one or more structural variants. In some cases, the one or more genomic variants comprise at least one structural variant. In some cases, the structural variant is about 30 bp to about 1,000 bp. In some cases, the structural variant is about 30 bp to about 50 bp, about 30 bp to about 100 bp, about 30 bp to about 500 bp, about 30 bp to about 750 bp, about 30 bp to about 1,000 bp, about 50 bp to about 100 bp, about 50 bp to about 500 bp, about 50 bp to about 750 bp, about 50 bp to about 1,000 bp, about 100 bp to about 500 bp, about 100 bp to about 750 bp, about 100 bp to about 1,000 bp, about 500 bp to about 750 bp, about 500 bp to about 1,000 bp, or about 750 bp to about 1,000 bp. In some cases, the structural variant is about 30 bp, about 50 bp, about 100 bp, about 500 bp, about 750 bp, or about 1,000 bp. In some cases, the structural variant is at least about 30 bp, about 50 bp, about 100 bp, about 500 bp, or about 750 bp. In some cases, the structural variant is at most about 50 bp, about 100 bp, about 500 bp, about 750 bp, or about 1,000 bp. In some cases, the structural variant is about 1 Kbp to about 1,000 Kbp. In some cases, the structural variant is about 1 Kbp to about 50 Kbp, about 1 Kbp to about 100 Kbp, about 1 Kbp to about 250 Kbp, about 1 Kbp to about 500 Kbp, about 1 Kbp to about 750 Kbp, about 1 Kbp to about 1,000 Kbp, about 50 Kbp to about 100 Kbp, about 50 Kbp to about 250 Kbp, about 50 Kbp to about 500 Kbp, about 50 Kbp to about 750 Kbp, about 50 Kbp to about 1,000 Kbp, about 100 Kbp to about 250 Kbp, about 100 Kbp to about 500 Kbp, about 100 Kbp to about 750 Kbp, about 100 Kbp to about 1,000 Kbp, about 250 Kbp to about 500 Kbp, about 250 Kbp to about 750 Kbp, about 250 Kbp to about 1,000 Kbp, about 500 Kbp to about 750 Kbp, about 500 Kbp to about 1,000 Kbp, or about 750 Kbp to about 1,000 Kbp. In some cases, the structural variant is about 1 Kbp, about 50 Kbp, about 100 Kbp, about 250 Kbp, about 500 Kbp, about 750 Kbp, or about 1,000 Kbp. In some cases, the structural variant is at least about 1 Kbp, about 50 Kbp, about 100 Kbp, about 250 Kbp, about 500 Kbp, or about 750 Kbp. In some cases, the structural variant is at most about 50 Kbp, about 100 Kbp, about 250 Kbp, about 500 Kbp, about 750 Kbp, or about 1,000 Kbp. In some cases, the structural variant is about 1 Mbp to about 10 Mbp. In some cases, the structural variant is at least about 1 Mbp. In some cases, the structural variant is at most about 10 Mbp. In some cases, the structural variant is about 1 Mbp to about 2 Mbp, about 1 Mbp to about 3 Mbp, about 1 Mbp to about 4 Mbp, about 1 Mbp to about 5 Mbp, about 1 Mbp to about 6 Mbp, about 1 Mbp to about 7 Mbp, about 1 Mbp to about 8 Mbp, about 1 Mbp to about 9 Mbp, about 1 Mbp to about 10 Mbp, about 2 Mbp to about 3 Mbp, about 2 Mbp to about 4 Mbp, about 2 Mbp to about 5 Mbp, about 2 Mbp to about 6 Mbp, about 2 Mbp to about 7 Mbp, about 2 Mbp to about 8 Mbp, about 2 Mbp to about 9 Mbp, about 2 Mbp to about 10 Mbp, about 3 Mbp to about 4 Mbp, about 3 Mbp to about 5 Mbp, about 3 Mbp to about 6 Mbp, about 3 Mbp to about 7 Mbp, about 3 Mbp to about 8 Mbp, about 3 Mbp to about 9 Mbp, about 3 Mbp to about 10 Mbp, about 4 Mbp to about 5 Mbp, about 4 Mbp to about 6 Mbp, about 4 Mbp to about 7 Mbp, about 4 Mbp to about 8 Mbp, about 4 Mbp to about 9 Mbp, about 4 Mbp to about 10 Mbp, about 5 Mbp to about 6 Mbp, about 5 Mbp to about 7 Mbp, about 5 Mbp to about 8 Mbp, about 5 Mbp to about 9 Mbp, about 5 Mbp to about 10 Mbp, about 6 Mbp to about 7 Mbp, about 6 Mbp to about 8 Mbp, about 6 Mbp to about 9 Mbp, about 6 Mbp to about 10 Mbp, about 7 Mbp to about 8 Mbp, about 7 Mbp to about 9 Mbp, about 7 Mbp to about 10 Mbp, about 8 Mbp to about 9 Mbp, about 8 Mbp to about 10 Mbp, or about 9 Mbp to about 10 Mbp. In some cases, the structural variant is about 1 Mbp, about 2 Mbp, about 3 Mbp, about 4 Mbp, about 5 Mbp, about 6 Mbp, about 7 Mbp, about 8 Mbp, about 9 Mbp, or about 10 Mbp.

As described elsewhere, the one or more target genomic variants may comprise one or more structural variants. In some cases, the one or more target genomic variants may comprise at least one structural variant. In some cases, the sample comprises about 1 target genomic variant to about 100 target genomic variants.

In some embodiments, the sample comprises RNA transcripts. In some embodiments, the sample comprises genomic DNA (gDNA). In some embodiments, the sample comprises gDNA and RNA transcripts.

In some embodiments, the sample comprises one or more target genomic variants. In some cases, the sample comprises about 1 target genomic variant to about 2 target genomic variants, about 1 target genomic variant to about 4 target genomic variants, about 1 target genomic variant to about 6 target genomic variants, about 1 target genomic variant to about 8 target genomic variants, about 1 target genomic variant to about 10 target genomic variants, about 1 target genomic variant to about 20 target genomic variants, about 1 target genomic variant to about 30 target genomic variants, about 1 target genomic variant to about 40 target genomic variants, about 1 target genomic variant to about 50 target genomic variants, about 1 target genomic variant to about 75 target genomic variants, about 1 target genomic variant to about 100 target genomic variants, about 2 target genomic variants to about 4 target genomic variants, about 2 target genomic variants to about 6 target genomic variants, about 2 target genomic variants to about 8 target genomic variants, about 2 target genomic variants to about 10 target genomic variants, about 2 target genomic variants to about 20 target genomic variants, about 2 target genomic variants to about 30 target genomic variants, about 2 target genomic variants to about 40 target genomic variants, about 2 target genomic variants to about 50 target genomic variants, about 2 target genomic variants to about 75 target genomic variants, about 2 target genomic variants to about 100 target genomic variants, about 4 target genomic variants to about 6 target genomic variants, about 4 target genomic variants to about 8 target genomic variants, about 4 target genomic variants to about 10 target genomic variants, about 4 target genomic variants to about 20 target genomic variants, about 4 target genomic variants to about 30 target genomic variants, about 4 target genomic variants to about 40 target genomic variants, about 4 target genomic variants to about 50 target genomic variants, about 4 target genomic variants to about 75 target genomic variants, about 4 target genomic variants to about 100 target genomic variants, about 6 target genomic variants to about 8 target genomic variants, about 6 target genomic variants to about 10 target genomic variants, about 6 target genomic variants to about 20 target genomic variants, about 6 target genomic variants to about 30 target genomic variants, about 6 target genomic variants to about 40 target genomic variants, about 6 target genomic variants to about 50 target genomic variants, about 6 target genomic variants to about 75 target genomic variants, about 6 target genomic variants to about 100 target genomic variants, about 8 target genomic variants to about 10 target genomic variants, about 8 target genomic variants to about 20 target genomic variants, about 8 target genomic variants to about 30 target genomic variants, about 8 target genomic variants to about 40 target genomic variants, about 8 target genomic variants to about 50 target genomic variants, about 8 target genomic variants to about 75 target genomic variants, about 8 target genomic variants to about 100 target genomic variants, about 10 target genomic variants to about 20 target genomic variants, about 10 target genomic variants to about 30 target genomic variants, about 10 target genomic variants to about 40 target genomic variants, about 10 target genomic variants to about 50 target genomic variants, about 10 target genomic variants to about 75 target genomic variants, about 10 target genomic variants to about 100 target genomic variants, about 20 target genomic variants to about 30 target genomic variants, about 20 target genomic variants to about 40 target genomic variants, about 20 target genomic variants to about 50 target genomic variants, about 20 target genomic variants to about 75 target genomic variants, about 20 target genomic variants to about 100 target genomic variants, about 30 target genomic variants to about 40 target genomic variants, about 30 target genomic variants to about 50 target genomic variants, about 30 target genomic variants to about 75 target genomic variants, about 30 target genomic variants to about 100 target genomic variants, about 40 target genomic variants to about 50 target genomic variants, about 40 target genomic variants to about 75 target genomic variants, about 40 target genomic variants to about 100 target genomic variants, about 50 target genomic variants to about 75 target genomic variants, about 50 target genomic variants to about 100 target genomic variants, or about 75 target genomic variants to about 100 target genomic variants. In some cases, the sample comprises about 1 target genomic variant, about 2 target genomic variants, about 4 target genomic variants, about 6 target genomic variants, about 8 target genomic variants, about 10 target genomic variants, about 20 target genomic variants, about 30 target genomic variants, about 40 target genomic variants, about 50 target genomic variants, about 75 target genomic variants, or about 100 target genomic variants. In some cases, the sample comprises at least about 1 target genomic variant, about 2 target genomic variants, about 4 target genomic variants, about 6 target genomic variants, about 8 target genomic variants, about 10 target genomic variants, about 20 target genomic variants, about 30 target genomic variants, about 40 target genomic variants, about 50 target genomic variants, or about 75 target genomic variants. In some cases, the sample comprises at most about 2 target genomic variants, about 4 target genomic variants, about 6 target genomic variants, about 8 target genomic variants, about 10 target genomic variants, about 20 target genomic variants, about 30 target genomic variants, about 40 target genomic variants, about 50 target genomic variants, about 75 target genomic variants, or about 100 target genomic variants.

### Target Enrichment Sample Preparation for Sequencing

In some aspect, the target enrichment sample preparation approach describe herein may comprise one or more genome editing technologies. In some cases, the genome editing technology is an endonuclease-based genome editing technology. In some cases, the endonuclease-based genome editing technology comprises zinc-finger nucleases (ZFNs), homing nucleases, transcription activator-like effector nucleases (TALENs), and/or clustered regularly interspersed short palindromic repeats (CRISPR)-Cas systems. In some cases, the target enrichment sample preparation approach may further comprise DNA amplification. In some cases, the target enrichment sample preparation approach may not comprise DNA amplification.

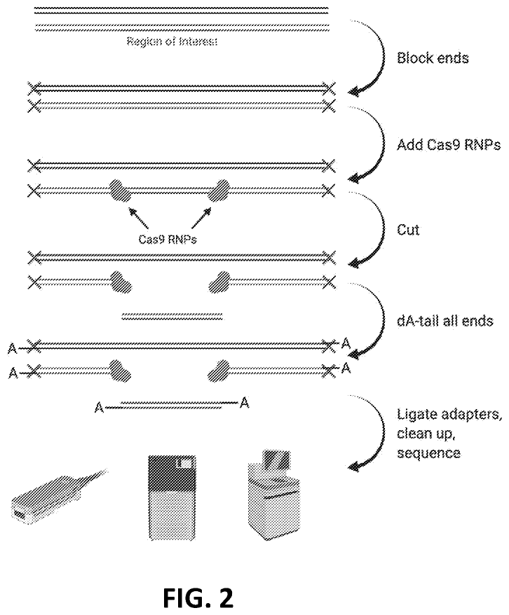

In some embodiments, the target enrichment sample preparation approach comprises preparing a sample for sequencing using a non-amplification-based, gene-editing based approach. In some case, the sample preparation comprises Cas-mediated PCR-free enrichment of said sample as shown in FIG. 2. Cas-mediated PCR-free enrichment of said sample may comprise extracting genomic DNA (gDNA) from said sample; dephosphorylating 5′ ends of the DNA to reduce ligation of sequencing adapters to non-target strands; adding Cas9 ribonucleoproteins (RNPs) comprising bound crRNA and tracrRNA to the gDNA to bind and cleave the region of interest (ROI); cleaving of gDNA by Cas9 to reveal blunt ends with ligatable 5′ phosphates; dA-tailing of gDNA in said sample to prepare blunt ends for sequencing adapter ligation; and ligating sequencing adapters to the Cas9 cut sides, wherein the Cas9 cut sides are 3′dA-tailed and 5′phosphorylated.

In some embodiments, a two RNP (ribonucleoprotein complex comprising Cas9-crRNA-tracrRNA) complexes, designed to excise a ROI, bind to sequences on the (+) and (−) strands, upstream and downstream of the ROI, respectively. The crRNAs confer specificity and ‘program’ the RNPs to bind to the specific sequences. Background DNA has been dephosphorylated (i.e. carries 5′-hydroxyl groups). Upon RNP binding, the duplex DNA is locally melted. crRNA hybridizes to the non-target DNA strand, which is complementary to the crRNA. Cas9 cleaves both of the DNA strands within the target site, 3 bp upstream of the PAM. Cleavage by Cas9 reveals 5′ phosphates at each end of the ROI. Existing ends of the same molecule, which carry 5′ hydroxyl groups, are considered non-target. The PAM-distal side is protected from ligation by Cas9 and/or the bound crRNA, whereas the PAM-proximal side is released for each RNP targeting the ROI. Because the RNPs here target the (+) strand and the (−) strand upstream and downstream of the ROI, the ROI is excised and both ends of the ROI are freed for dA-tailing and adapter ligation. Adapter ligation to the ROI results in directionality of the expected reads.

In some cases, an alternative to Cas9 may be used in the CRISPR-Cas system, wherein the alternative to Cas9 may be Cas3, Cas4, Cas5, Cas8a, Cas8b, Cas8c, Cas10, Cas10d, Cas13a, Cas13b, Cas13c, Cse1, Cse2, Csy1, Csy2, Csy3, Csm2, Cmr5, Csx10, Csx10, Csf1, Csn2, Cpf1, C2c1, or C2c3.

In some cases, the target enrichment sample preparation comprises preparing a sample for sequencing using the PacBio® sequencing system. In such cases, genomic DNA (gDNA) is dephosphorylated and then subjected to Cas-mediated PCR-free enrichment as described herein. Following the cleavage reaction, SMRTbell® adapters are ligated to the blunt template ends, forming SMRTbell® templates. In the final step, unligated DNA is eliminated by exonuclease digestion and then prepared for sequencing by annealing to the Sequencing Primers and binding to the polymerase.

In some cases, the target enrichment sample preparation comprises preparing a sample for sequencing using Illumina® sequencing system. In such embodiments, gDNA is dephosphorylated and then filled in using biotinylated nucleotides. The gDNA is then subjected to Cas-mediated PCR-free enrichment as described herein. After the cleavage reaction, non-target gDNA is removed using streptavidin beads. The target gDNA is then fragmented to the appropriate size, end-repaired, and dA-tailed. Illumina® adapters are ligated to the end-repaired, dA-tailed target gDNA, and is then ready for sequencing.

crRNA Probes

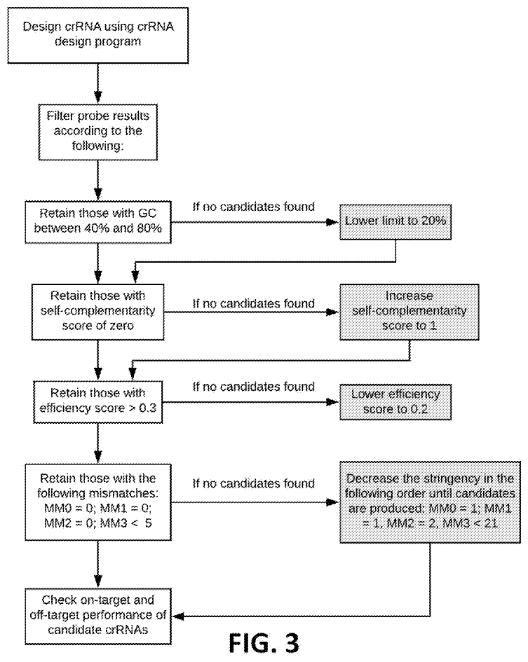

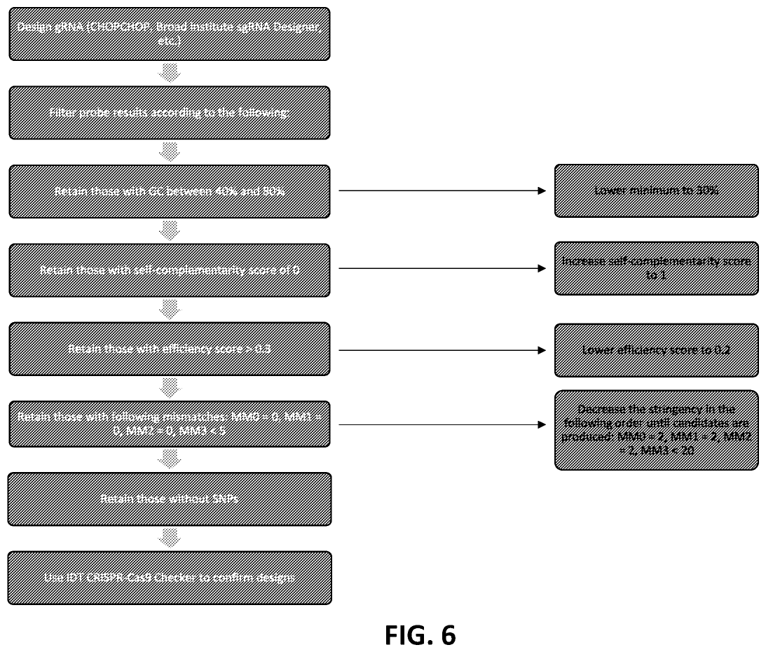

In one aspect, provided herein are systems and methods to design crRNAs for use with the systems and methods described herein. In some embodiments as shown in FIG. 3 and FIG. 6, preliminary crRNA probes are designed using available guide RNA (gRNA) tools. Exemplary gRNA design tools include CHOPCHOP program, based on ONT recommended design options, and Broad Institute sgRNA Designer. In some cases, the preliminary crRNA probes are designed from Benchling probe design tool and/or CRISPOR probe design tool.

The preliminary crRNA probes are filtered using one or more approaches as shown in FIG. 3 and FIG. 6. One filter approach is to retain preliminary crRNA probes with a GC content between about 40% and about 80%. If no candidates are obtained, the lower limit of the range is lowered to a GC content between about 20% and about 80%. Another filter approach is to retain preliminary crRNA probes with a self-complementarity score of zero. If no candidates are obtained, the self-complementarity score is increased to 1. Another filter approach is to retain preliminary crRNA probes with an efficiency score greater than 0.3. If no candidates are obtained, the efficiency score is lowered to greater than 0.2. Another filter approach is to retain preliminary crRNA probes with the following mismatches: MM0=0, MM1=0, MM2=0, and MM3<5. In no candidates are obtained, the stringency of the mismatches is decreased in the following order: MM0=1, MM1=1, MM2<2 and MM3<10, until candidates are produced. In another embodiment, the stringency of the mismatches is decreased in the following order: MM0<1, MM1<2, MM2<2 and MM3<21, until candidates are produced. In some cases, candidates are further filtered by retaining candidates without any single nucleotide polymorphisms (SNPs). In some cases, ambiguous bases are introduced at any position to increase on-target performance.

In some cases, two or more of the approaches are used. In some cases, three or more of the approaches are used. In some cases, four approaches are used. In some cases, the following approaches are used in the following order: GC content, self-complementarity score, efficiency score and mismatches. After filtering the preliminary crRNA probes using one or more of the filter approaches, the on-target and off-target performance of candidate crRNA probes are confirmed using a guide RNA check tool. Examples of guide RNA check tools include IDT CRISPR-Cas9 gRNA checker, Cas-OFFinder, Dharmacon's CRISPR specificity analysis tool, Synthego's CRISPR specificity analysis tool, or a combination thereof.

Candidate crRNA probes obtained using the methods provided herein are more likely to cut the target genomic region of interest than crRNA probes obtained using other methods. In some cases, the probability that a candidate crRNA probe will cut a target is about 60% to about 99.9%. In some cases, the probability that a candidate crRNA probe will cut a target is at least about 60%. In some cases, the probability that a candidate crRNA probe will cut a target is at most about 99.9%. In some cases, the probability that a candidate crRNA probe will cut a target is about 60% to about 65%, about 60% to about 70%, about 60% to about 75%, about 60% to about 80%, about 60% to about 85%, about 60% to about 90%, about 60% to about 95%, about 60% to about 99.9%, about 65% to about 70%, about 65% to about 75%, about 65% to about 80%, about 65% to about 85%, about 65% to about 90%, about 65% to about 95%, about 65% to about 99.9%, about 70% to about 75%, about 70% to about 80%, about 70% to about 85%, about 70% to about 90%, about 70% to about 95%, about 70% to about 99.9%, about 75% to about 80%, about 75% to about 85%, about 75% to about 90%, about 75% to about 95%, about 75% to about 99.9%, about 80% to about 85%, about 80% to about 90%, about 80% to about 95%, about 80% to about 99.9%, about 85% to about 90%, about 85% to about 95%, about 85% to about 99.9%, about 90% to about 95%, about 90% to about 99.9%, or about 95% to about 99.9%. In some cases, the probability that a candidate crRNA probe will cut a target is about 60%, about 65%, about 70%, about 75%, about 80%, about 85%, about 90%, about 95%, or about 99.9%.

Provided herein are exemplary target genomic sequences (e.g., a protospacer) to which crRNA probes may be hybridizable for use with the systems and methods described herein. A guide RNA can target a nucleic acid sequence of or of about 20 nucleotides. A target nucleic acid can be less than or less than about 20 nucleotides. A target nucleic acid can be at least or at least about 5, 10, 15, 16, 17, 18, 19, 20, 21, 22, 23, 24, 25, 30 or more nucleotides. A target nucleic acid can be at most or at most about 5, 10, 15, 16, 17, 18, 19, 20, 21, 22, 23, 24, 25, 30 or more nucleotides in length. A target nucleic acid sequence can be or can be about 20 bases immediately 5′ of the first nucleotide of the PAM. A guide RNA can target the nucleic acid sequence. A guiding polynucleic acid, such as a guide RNA, can bind to a genomic sequence with at least or at least about 50%, 60%, 65%, 70%, 75%, 80%, 85%, 90%, 95%, 98%, or up to about 100% sequence identity and/or sequence similarity to any of the sequences of the tables below.

TABLE 1
Target sequences for ABCD1 gene

| SEQ ID NOS | Target sequence |
|---|---|
| 1 | TCAGCAACAACGTGACCCAGTGG |
| 2 | GTCATGACGAAGCAGAACCCTGG |
| 3 | TCATGACGAAGCAGAACCCTGGG |
| 4 | GTTCTGTTGCAAAACCCACAAGG |
| 5 | TTGGAGGCCATTAGTTAGTGCGG |
| 6 | GAGGCCATTAGTTAGTGCGGAGG |
| 7 | AGGCCATTAGTTAGTGCGGAGGG |
| 8 | CAGGTCTCCTGATTTACCTCGGG |
| 9 | CTTGCCCCATCTCGCATACCCGG |
| 10 | TGAGGGGTAACCACCTGTGCCGG |
| 11 | CCAGAAACCCGAGGTAAATCAGG |
| 12 | AAGTGTTACAAAGGGTCTCCAGG |
| 13 | CGGGTATGCGAGATGGGGCAAGG |
| 14 | TCATGGGGCCCCTGCGCGCAGGG |
| 15 | CGCAGGGCCACATATGCTCAGGG |
| 16 | GCGCAGGGCCACATATGCTCAGG |
| 17 | TTACCCCTCACCGCTCGCAGCGG |
| 18 | CTGAGGTAAGCTAAAGACCACGG |
| 19 | TCCAGGTAGACAGCTGTTCAAGG |
| 20 | GGGGCACTAAAGTGTTACAAAGG |
| 21 | CGGGGTTTATGATCAAGCATGGG |
| 22 | ACGGGGTTTATGATCAAGCATGG |

TABLE 2
Target sequences for ACADM gene

| SEQ ID NOS | Target sequence |
|---|---|
| 23 | AAGGGGTTACAATAGGCATATGG |
| 24 | CGTGTTGTAATCATCATAGAAGG |
| 25 | TGTATGGAGGGATTGAACACAGG |
| 26 | GATTACAACACGTGACCTCAGGG |
| 27 | CTTAATCACATGGTCCTCGGGGG |
| 28 | GTTCAATCCCTCCATACAAGTGG |
| 29 | ACTTAATCACATGGTCCTCGGGG |
| 30 | TGATTACAACACGTGACCTCAGG |
| 31 | AACTTAATCACATGGTCCTCGGG |
| 32 | TAGAATGAGGCCCAGCAACCAGG |
| 33 | ACAGTCATTTATTGCTACTAGGG |
| 34 | GAGGGATGCCAAAATCTATCAGG |
| 35 | GATAGGACTCTAATCTCACAGGG |

TABLE 3
Target sequences for ACADVL gene

| SEQ ID NOS | Target sequence |
|---|---|
| 36 | GGGTCTTGCCAAACGGCCAG |
| 37 | GGGGTCTTGCCAAACGGCCA |
| 38 | AGCACACCCCGATTCTCAGG |
| 39 | CACACCCCGATTCTCAGGAG |
| 40 | GGGAGCACACCCCGATTCTC |
| 41 | GAGGCCCGCAAGTATGCCAG |
| 42 | TCCCGCACTAGGTCCTGCAC |
| 43 | GACGTCCACCCATGTGCTGC |

TABLE 4
Target sequences for AFF2 gene

| SEQ ID NOS | Target sequence |
|---|---|
| 44 | GTGATACCATGTATGCCACGTGG |
| 45 | GATGCTAAGTGTACACCACGAGG |
| 46 | CTAACGAAAGACACCAACTG |
| 47 | CCTTCCCTAAGTGAACCAAGGGG |
| 48 | TCGTATCTAACACTCCCCTGGGG |
| 49 | CTAGTATTATCGATACCCAGAGG |
| 50 | GTAGGTTTCATACCACAATGTGG |
| 51 | GGTCTCTATCAAGTTCAAGGTGG |
| 52 | TCTGCCACTAAAGCAACCAGCGG |
| 53 | GTTCTCATGATCTCGCAGAGGGG |
| 54 | TGAGCACAACTTCAACTGGGGGG |
| 55 | GTCATAATCACAGTACATCGTGG |
| 56 | ACCATACACCAAATGCCACGTGG |
| 57 | GTGCTACTGCCACCTCACGGTGG |
| 58 | TTACGCCAGCACAAAAACGTGGG |
| 59 | TGCCTGCGATAATTACAGAGTGG |
| 60 | GAACTGTAAATATAGATACGTGG |
| 61 | GAACTCATATGCAAACCTCGTGG |
| 62 | TCTAACTAAGGATCAGCACAGGG |
| 63 | CGACACATTGGATGAAACGTGGG |
| 64 | TATCAAAAATACCCACAGCGGGG |
| 65 | TGTTACCTGGAGTACTACGATGG |
| 66 | TAGATGTCCACCATACTCAGAGG |
| 67 | CAACTTGTTCAGTATGACGAGGG |
| 68 | CAAGTTCCCTCAGATCGCTGAGG |
| 69 | ACCACAGTCCCTAATTACCGTGG |
| 70 | AGTGGCTTGGTACAACAATGAGG |
| 71 | GCTGGAGTATATAATCCCCGGGG |
| 72 | ATATGTGATATACTACCCTGTGG |
| 73 | TGGGCGTAAGAAACTAATTGAGG |
| 74 | GCTGGTGACGAGGTTAGACGTGG |
| 75 | CGTCTTCGCAGTAATTCTGGAGG |
| 76 | CCTTACAATGTATGTCCCAGTGG |
| 77 | GATAATGCTCATATGTGACAGGG |
| 78 | TGGGACCTTTGCAATACACAGGG |
| 79 | CTTTTAGTGATACTTCCACGTGG |
| 80 | ACCATACTGTCACAACAAGTGGG |
| 81 | GGCTATGTAGATACCTGTGGAGG |
| 82 | GATCACTAATCGCCCACCCAGGG |
| 83 | GAAAGTCCTTAAAGACCCCGTGG |
| 84 | GAGCATTGTTAGTGAACTGGGGG |
| 85 | ACTGACATAAATGCCGTGGGTGG |
| 86 | GGTATTATAGTTCATCAGGGTGG |
| 87 | CTCTATTGTGACATGCAAGGTGG |
| 88 | TTTTCATCGAGTGTGCGTCGTGG |
| 89 | ATAGCGAAATCTATCTCACGAGG |
| 90 | GGCACTTGTACATTTAACGTGGG |
| 91 | ATGAAAAACTCAGCTTACGGTGG |
| 92 | GGTACCAAGATCTAAAATGGGGG |
| 93 | ATCGTTTATATAATACCCAGAGG |
| 94 | CCACCTTGAAGAAATACCGGGGG |
| 95 | TATGTGATATACTACCCTGTGGG |
| 96 | GCTTGTTATAAATTACACCAAGG |
| 97 | GGATTCACTCTGGTAAAGCAGGG |
| 98 | TGTGCGCAGATTCTCCGATGAGG |
| 99 | GGGAGTTACATACCCGTACAGGG |
| 100 | CTCAAGATACCCCATGACAGAGG |
| 101 | GATAAGAAGCAATCCACTGGGGG |
| 102 | TACGTAGAGAGTAAGTCCAGAGG |
| 103 | GAGACAACGTTACAAAAGCGCGG |
| 104 | ATATCTTATCTCCTACACCAAGG |
| 105 | GCTCCCCTGATGGTAAGACGTGG |
| 106 | CAGTTCCCAGCAAATAAACGAGG |
| 107 | TGACCACGTCTTACCATCAGGGG |
| 108 | ACGGGCAACTGAGGTAATGGGGG |
| 109 | TCTGAACACTAATGTCACAAGGG |
| 110 | TAAGTTTGACAGCTAACCAGTGG |
| 111 | AATAGTCGGTTTCTTCAATGTGG |
| 112 | GACACACTTAAATAAGCACGTGG |
| 113 | TAGGCACTTGTCCAGAAACGAGG |
| 114 | CACGCAATGAGATAATTCTG |
| 115 | TCATAGGAAATGGCTCGCTGCGG |
| 116 | GGTATAATAGGCCAGTCTTGCGG |
| 117 | GGTGCTTATCCACTATTCAGGGG |
| 118 | ATAACTCAGTAGATCAACCTGGG |
| 119 | GTCCTTGGAGAACCCTTCGGAGG |
| 120 | ATGCTCTATTGTGACATGCAAGG |
| 121 | CCGTTAGGCTTCATAAACCATGG |
| 122 | ATGTTTCTGATGAAGTGCCGTGG |
| 123 | CAGGGTATTATAGTTCATCAGGG |
| 124 | TATCTCAAGAAGGTAACTCGGGG |
| 125 | AGGTTCTCATGATCTCGCAGAGG |
| 126 | GGGAATGAACACCATACCGAAGG |
| 127 | TCTAACCTAAATGGTCTGTGTGG |
| 128 | ACCTGTCCTATGGAGTATGGTGG |
| 129 | TCACACCCTTGTTAGACCAGTGG |
| 130 | CATAATCCTCTACTACGATG |
| 131 | CTTCTCTATAAATCTCAGGGAGG |
| 132 | TTACCAAAAAGGCTTATCCGTGG |
| 133 | AAATGCGGGGATAATTCCAAAGG |
| 134 | GGAGTTATAACAATTGTCCG |
| 135 | GAGTTGTCAAACCATTATCGTGG |
| 136 | TAGTGCCATCGCTTTAAGGGTGG |
| 137 | GCTAAAACACTGGTAACTCAGGG |
| 138 | GATGTGCTACTTCTACCTGAAGG |
| 139 | TATGAATAACAATGGTACGAAGG |
| 140 | ATTCATAGAGTGGTTGTCAGGGG |
| 141 | ATGATGATAGAACCTTAAGCCGG |
| 142 | GCTTGACATTAGATAGACCATGG |
| 143 | TCTGGAAGTTGGAACTCCGTAGG |

TABLE 5
Target sequences for ALPL gene

| SEQ ID NOS | Target sequence |
|---|---|
| 144 | TGATACCATCTTAAGTCTCCTGG |
| 145 | CACTTAGGTGATTAAGGGCTTGG |
| 146 | CAACATGCACGTACTAGGCATGG |
| 147 | CTGCCCAAGGCTTAGCTAGGTGG |
| 148 | AGGCCACCTAGCTAAGCCTTGGG |
| 149 | AATTTCCCCATTGTGCGTCTTGG |
| 150 | TTTACAACCCTTTGACCACCAGG |
| 151 | TAGGTCCCTCTGCTAAACAGGGG |
| 152 | GTACCTGCAAGTCCTGTCACAGG |
| 153 | GCATGGAACCTGGTGGTCAAAGG |

TABLE 6
Target sequences for APC gene

| SEQ ID NOS | Target sequence |
|---|---|
| 154 | TATATCAGGCATTGTAACAC |
| 155 | TGTGTATGGCCCCACAAAGA |
| 156 | ATCTTTGTGGGGCCATACAC |
| 157 | TCGTTATAACACCAGTTCTG |
| 158 | CGTTATAACACCAGTTCTGT |
| 159 | AGATCTAGTTAGTTCTACAA |
| 160 | GTACAATCCAATGACATCTG |
| 161 | AGATGTCATTGGATTGTACC |
| 162 | CCAGTCATGTTTGATATACT |
| 163 | GTCTCCTGCACTACAAGACT |
| 164 | AAGTTCACAACTAACTGGTT |
| 165 | CTCTTGGAGGTTGTAAACTC |
| 166 | CCTAGTATATCAAACATGAC |
| 167 | ATTAGGGTTTAGTGTACTAA |
| 168 | TTAGTCCTCTACCTTACTGG |
| 169 | GCCTATTTTGTGATTGCCAA |
| 170 | CATGAGAATAACGACCTCAA |
| 171 | GGTGACCCCCAGTAAGGTAG |
| 172 | CGACCTCAAAGGATATCATG |
| 173 | TAAGTGTCCATCAACTAGGG |
| 174 | CCATGCAGTTAAGAGGTACC |
| 175 | AACCTTTGCTTACATGCCTA |
| 176 | GGTGGTACTTACCCTTCCAT |
| 177 | TTGTTAATAGAGCTTACTAC |
| 178 | TGTCAACTATATTACCCTAT |
| 179 | CATCCAGGCTTATAATCTCC |
| 180 | TTTGCATGGATGCACCATAT |
| 181 | TGCACCATATAGGTTCCATG |
| 182 | TTTACGATCAATGTCCATTT |
| 183 | TTGGCCTCATGGAACCTATA |
| 184 | GTAGATTAGTTAAGTGGCTC |
| 185 | AATGAGGGGGATTAGCCACA |
| 186 | CAGGTAGTATATTAGTCACC |
| 187 | CTGGTGACTAATATACTACC |
| 188 | AAGACATCCAGACTGTCGCA |
| 189 | AATGCTTGGTACTCATGATA |
| 190 | TCTAAACTCATTTGGCCCAC |
| 191 | GAATGCGTATCTAACAAGGG |
| 192 | ATGCGTATCTAACAAGGGTG |
| 193 | AATGCGTATCTAACAAGGGT |
| 194 | CTTAACTTAGACCTGGGATT |
| 195 | TTGTTAGATACGCATTCATC |
| 196 | CTCATTTGTAGCTATCAAGC |
| 197 | ATGGGTCATCTAATTAGAGT |
| 198 | TAGCTACAAATGAGGACCAC |
| 199 | ACCAGTGAGGGACGGGCAAT |
| 200 | TGGTTGGCACTCTTACTTAC |
| 201 | TGGTACAAATAGCCAAGGTC |
| 202 | CTCTAGGTCAGATACAACTC |

TABLE 7
Target sequences for ASPA gene

| SEQ ID NOS | Target sequence |
|---|---|
| 203 | CTATGTAAGTTCACATGATGTGG |
| 204 | AACCTGGCGTTACTAGTACATGG |
| 205 | CACTAACTACAGTTCTGAGTAGG |
| 206 | TTGGATCTGCCTTCTCAACCAGG |
| 207 | AAATTCTGAGTCCGTAATCCAGG |
| 208 | TTAGCTAAGTGACAGGTCTCAGG |
| 209 | ACTAAGTTCGCAGTCTCACATGG |

TABLE 8
Target sequences for ATM gene

| SEQ ID NOS | Target sequence |
|---|---|
| 210 | AATTGCGAGGACAACTGTCT |
| 211 | GAATTGCGAGGACAACTGTC |
| 212 | GATCACAACTGGGTAAGGGT |
| 213 | ATCACAACTGGGTAAGGGTA |
| 214 | CAGGTCCAATCTTCCTATGA |
| 215 | CTTCATAGGAAGATTGGACC |
| 216 | GATTCTGTGAGATTGAATCG |
| 217 | GTTAAACTGTCAGGTCACTT |
| 218 | CATCGTCAAGGAGTTGACAG |
| 219 | GCCATGATGAGTTGGTCCAA |
| 220 | AAAGGCTAGTATAAGCCCAA |
| 221 | ATGCATAAGTAGCTCCTAGA |
| 222 | AGTGATACTCTAGGGCAAAC |
| 223 | GATGCATAAGTAGCTCCTAG |
| 224 | GGGCAATACTCTCTTGGTAT |
| 225 | GCCTTTGGACCAACTCATCA |
| 226 | CCCTAGAGTATCACTTGTTA |
| 227 | GGAACTTTATTGGCTGGAAC |
| 228 | TAGTTAGGAACTTTATTGGC |
| 229 | ACCGAATTCACTCCTTTGAA |
| 230 | AGGAGTGAATTCGGTAGCCA |
| 231 | GTTCTAATTAGGGACTCACC |
| 232 | GACCCAACTTGCTACTCGCT |
| 233 | TTCAGGTTGAGTGGATAGTC |
| 234 | GCTCTACCTCCACATACACT |
| 235 | GGCTCTACCTCCACATACAC |
| 236 | CGAGTAGCAAGTTGGGTCCT |
| 237 | TGGCCTAGCGAGTAGCAAGT |
| 238 | GCGTAACACCCACATATTTA |
| 239 | TCCCATTAGGCATAACCTAA |
| 240 | GGACTCAACTAATTGGTGTT |
| 241 | ATATGTGGGTGTTACGCAAA |
| 242 | CCAAATCCCTAACAGAGTTA |
| 243 | CCTTAACTCTGTTAGGGATT |
| 244 | GCTTCAAGCTGACTTTAACC |
| 245 | CAGGTGATTTCTCCATCCCG |
| 246 | TAGATTTAGTGACCACGGGA |
| 247 | CACGGGATGGAGAAATCACC |
| 248 | TCAGCTTGAAGCTCTCGTGA |
| 249 | GTGTTTAGATTTAGTGACCA |
| 250 | CTATAATCTAGTAGGATCAC |
| 251 | ATCTAGTAGGATCACAGGAT |
| 252 | CTGTGATCCTACTAGATTAT |
| 253 | CATGCTTGAAGGCTCATTAT |
| 254 | ACAAGTGGACAAGTCAGATC |
| 255 | GCCAAGCTGTTTCTATCCAA |
| 256 | TACACGATTCCTGACATCAA |
| 257 | GCTACTTATGTGTAGAGCAC |
| 258 | CTTTGCAGTTACCATAGGAG |
| 259 | TAGAGTATCTAACCCAACGT |
| 260 | GGGCATGTAGAATACTTATT |
| 261 | GTGAATTTATATACCTACGT |
| 262 | ATCTTAATGAACCACTCATA |
| 263 | AGACAGTCACGGATATTATA |
| 264 | TGGAGTACAACCCATATGAG |
| 265 | AAAGATGCCTCGGTTCATAA |
| 266 | CGGTTCATAAAGGTGCACAC |
| 267 | GCCCCACCCTTATTGACCAC |
| 268 | AAGATGAGTAACAGTCCATC |
| 269 | CATTAAGCCTGTGGTCAATA |
| 270 | GGCATTAACCATTAAGCCTG |
| 271 | AAGCCTGTGGTCAATAAGGG |
| 272 | AATAGGTCCCAATAATACGT |
| 273 | GTGTGCACCTTTATGAACCG |
| 274 | GGAGGTTGGTTGCACACCAC |
| 275 | ACTCACCATTAGTAGTATAC |

TABLE 9
Target sequences for ATM gene

| SEQ ID NOS | Target sequence |
|---|---|
| 276 | AATGGGATCCCTTCCTAAGG |
| 277 | GTACCAAGACGTGGATATGG |
| 278 | GGTACCAAGACGTGGATATG |
| 279 | GATGTGTAGGTACCAAGACG |
| 280 | CTGGAACCTATGATCAGGCA |
| 281 | TAGGTACCAAGACGTGGATA |
| 282 | AGGTACCAAGACGTGGATAT |
| 283 | TGTCTCTGGAACCTATGATC |
| 284 | TGTCACAAGAGGTGCTTACA |
| 285 | ATGACTCTGAACTGCCCACC |

TABLE 10
Target sequences for ATXN1 gene

| SEQ ID NOS | Target sequence |
|---|---|
| 286 | GGCAACTCAGATACTCACGTGGG |
| 287 | CCCCAATAGAGATTGCCCTGTGG |
| 288 | ACATCAGAACATGAGCACCGGGG |
| 289 | CAGGTGAGCGTACTGCACGGGGG |
| 290 | CGGGTCAAACCCCATCACAGTGG |
| 291 | TAAGTTGTCGTTGATCACAGGGG |
| 292 | GAAACAGGTATGATGCATGGGGG |
| 293 | GTCCACTTTATAAATCCCAGAGG |
| 294 | CTAAAACTTCTCATGCAAGGCGG |
| 295 | TTGCGATCAGAAACACAAGGAGG |
| 296 | TATAAGTGTTAAGGGCACCGGGG |
| 297 | AAGGTTACTCGGGTTCACAGAGG |
| 298 | CGAGACCTGACCATACTGTGGGG |
| 299 | GTAGTTCGAACACCCAACCAGGG |
| 300 | CAGAGTTTCGTACAGCAGCGTGG |
| 301 | TTGTAAACCAAGCTCCACCGAGG |
| 302 | TGTCACTTTAGACCAACCCGAGG |
| 303 | CCTGATCCAGTAAGTCACGGAGG |
| 304 | TTTCTGATGAGAGATCGCGGGGG |
| 305 | GTGTTTATGAACTCGCCAGGAGG |
| 306 | GTAGAAGATAGAATTCATTGGGG |
| 307 | AGTCTCAGCACATGACAACGTGG |
| 308 | GGTATACGTTCCAACCTCAGAGG |
| 309 | TGTATCACTACAGTTAAACGGGG |
| 310 | TGTCGGTAAATATTGCAAAGTGG |
| 311 | CAACCCCACATATCAAACCGTGG |
| 312 | GGACTGGTGGAACAACCGGGAGG |
| 313 | TGATCGCTGTAAGACCAAAGAGG |
| 314 | ACCACGTTGCAATATCTGGGAGG |
| 315 | GCAATGTGATTCTACACCCGGGG |
| 316 | AATGATTTGTCACTTTACCGAGG |
| 317 | ACATATACCTTACCCCAGCGAGG |
| 318 | TGTAGTAGAGCACACCAAGGGGG |
| 319 | TGGCCGGTTCCTATTCCATGGGG |
| 320 | GTGAACGCACCTGATCCATGAGG |
| 321 | GATCAATTCCAGGAGTTACGGGG |
| 322 | TGGTACTCTTGAGGTAAACGGGG |
| 323 | GGGCGATGAGGTAATTTGAGCGG |
| 324 | TCCAGAGATAAACTCCTCGGGGG |
| 325 | ACTAAGATTCATCTACCACGTGG |
| 326 | CATCTGGGTAGAGTACGTGGTGG |
| 327 | GCTCATTGTATCAACCAGTGTGG |
| 328 | GTACACTTTAAGATGCCACATGG |
| 329 | CTGTGCGATTGCCCACAAGGAGG |
| 330 | TTAGAAAGCACGTCCCAACGTGG |
| 331 | AGGCTATTATCTCATAACCGGGG |
| 332 | GTATCACTACAGTTAAACGGGGG |
| 333 | TTGACCGCCAAAACCAACCAGGG |
| 334 | GCTCATCGTAACTAAACCAGTGG |
| 335 | GCCGCAAACCAAGACATGTGAGG |
| 336 | TCCACATTCACTATTCCGTGTGG |
| 337 | ATCCGTAATAGATTGCTGAGAGG |
| 338 | GCGCAGCACTGGAACCACGTAGG |
| 339 | ATTAAAGTAGACCCCCCCAAGGG |
| 340 | ACGTCCTCTGATGAAAAGGGCGG |
| 341 | ACCTCCCTCTTGACAAACGGGGG |
| 342 | GCATCCAGATGCGACCCCCGAGG |
| 343 | TACACACAGCAGAGATCACGGGG |
| 344 | TATATCCAGGAGTTTGTAGGGGG |
| 345 | GTGACATTGTGATACCCCAAAGG |
| 346 | ATCTCCGGGGTATAAGACATAGG |
| 347 | GAAACTCACAAATGGCTACGAGG |
| 348 | ACTATTCCGTGTGGTGACAGGGG |
| 349 | CTTCACTCTAATGAGATACGTGG |
| 350 | CGGTGCATAGACACTTAAGGTGG |
| 351 | GAGCGTGACAGGAACCCCAAGGG |
| 352 | AGTACATGCATCATCCCAAGAGG |
| 353 | GTGCACAGGTCGTCTCTCAGGGG |
| 354 | AAGTTCTACAATGACACAGGTGG |
| 355 | ATACGTGGAACAAATTACTGGGG |
| 356 | ATTGAGGAGACACCTACCAGTGG |
| 357 | TCTGTGACCCTAATCTACGTGGG |
| 358 | TTAGGAAGTTCGATCCAACAGGG |
| 359 | TCAGGAGGAGAATGGTCGCGGGG |
| 360 | GTTTAAGATGATGTAATCGAGGG |
| 361 | CAGGATACATAACCCACAGGAGG |
| 362 | CTGTATACCACGAGACATGGAGG |
| 363 | GATACTTTTGGTAAACAACGTGG |
| 364 | ACTAGTTAGGATAGATGACAAGG |
| 365 | GAACACCGTAATGATGGCAGTGG |
| 366 | GCGTATCAGCACCACCCCCGTGG |
| 367 | GGTTCTAAGCTCAACTCCAGAGG |
| 368 | CGTCAAGTATGAAACCGGTGGGG |
| 369 | GCCACGACCAGATATCAGCTGGG |
| 370 | ATGATGGCACCCGAAGACAGTGG |
| 371 | GGGTGAAACAATTCCTTACGGGG |
| 372 | TCGCAACTTCAGCATAACAACGG |
| 373 | CATAGTATGTCAAACTCACGAGG |
| 374 | TCATCATCTTTTTGTCAACGGGG |
| 375 | CTAAGATTCATCTACCACGTGGG |
| 376 | GAATGGCGGTGATAAGCTAGAGG |
| 377 | ATTCGTTTGAGGGTGTTGGGAGG |
| 378 | AACCTACTTCCTACCCAAGGAGG |
| 379 | TCTAAGACGTTGCAGCAGTGGGG |
| 380 | GCTACTACCATATGCCCGAGGGG |
| 381 | GAAACTCTATGATACCCCAAAGG |
| 382 | CAGGTCATATACAACATCCGTGG |
| 383 | GCTATTTGAAAATCCCACGTAGG |
| 384 | AAGTGACGTTTTGGTCCTGGAGG |
| 385 | GTAATTCCCAAGACGCATGGTGG |

TABLE 11
Target sequences for ATXN2 gene

| SEQ ID NOS | Target sequence |
|---|---|
| 386 | GGTAACTCCACAATTCTACGAGG |
| 387 | TATGTGGTTCTGTACTTACGTGG |
| 388 | ACGATGATCTGGTATCCTGGTGG |
| 389 | GATACGCACAAACCTAAGTGAGG |
| 390 | ACCGTGGGTAAAGTCCTCTGGGG |
| 391 | TACCCCTTTAACCGGCACGGGGG |
| 392 | GTCCAAGATAATGACCTGAGAGG |
| 393 | AGGCCAGGGATCTATCATCAGGG |
| 394 | TGATGACCACGTTCCCCCCGAGG |
| 395 | TGTATCCATCTTCACGAGGGTGG |
| 396 | AAGTTGTATAGGCACTGACTTGG |
| 397 | GAACTTGGTACAGAGACGCTGGG |
| 398 | GTAAGTATGAGGATCTCGAATGG |
| 399 | TGACGATCAGTTCAGCATCAGGG |
| 400 | CAGATCACGTGTATTTGAGAGGG |
| 401 | TGTTAAATAGTGCGCCAGTGAGG |
| 402 | GATGACCACGTTCCCCCCGAGGG |
| 403 | GTGCCGCCAGAGCTTACCAAGGG |
| 404 | CCAGATCACGTGTATTTGAGAGG |
| 405 | GGAATCATCAGGGTCTGTCGGGG |
| 406 | ATTGTTTTGAGATCGTGCCCAGG |
| 407 | TGCCCAGTACAAAGCTCGAGTGG |
| 408 | AGTTTAGGCCCAAAGCTCCACGG |
| 409 | GGGACTGAATATGCGTGCAAAGG |
| 410 | TATAGTTACTTATCAACTGGAGG |
| 411 | ATTGCGTGGAGTAAGCTGGTGGG |
| 412 | GTAGTGTTGTACGATATCATGGG |
| 413 | AATCAAACAGCGTGTAACAGAGG |
| 414 | ACGGGTAGACATAATAGTTGGGG |
| 415 | AATGTGCGAACTTTAGACCTTGG |
| 416 | ATATTCAACGATTCCAAGGTCGG |
| 417 | AACCCCTCCCAACACGCGTGGGG |
| 418 | TTTAGGTGTGAACGTTGGAGGGG |
| 419 | CGTCTGTGGAAACCCCGAGTCGG |
| 420 | CATATGTTTTAGTGGTATCGGGG |
| 421 | TTGCGTGGAGTAAGCTGGTGGGG |
| 422 | TCACAGCTCATATGACGTAAAGG |
| 423 | GTAAGGTAGATTCTTCACGTTGG |
| 424 | CGTGGAGTAAGCTGGTGGGGTGG |
| 425 | TATCGCATCGTCAGAACACATGG |
| 426 | TCTGACCCAGAATTTGACGATGG |
| 427 | AACTTGCGAGTATATTAACAAGG |
| 428 | ACCGTGAGTTTATTCTCCCAGGG |
| 429 | AGACAGAATTCACCGCGTATGGG |
| 430 | TCACAGGATTATATGTACCCTGG |
| 431 | AAGCATCCTAAGTGGTGTGTGGG |
| 432 | TAAATACCGGTAAACTTGCAAGG |
| 433 | CCACGTAAATGGTGTGCAGAGGG |
| 434 | TGGGGTGGGTTGGTATACGCCGG |
| 435 | GATATGACGTCTTCATGCCAGGG |
| 436 | GAACCCCTCCCAACACGCGTGGG |
| 437 | ATCTCGAGTGATTGAATCTGAGG |
| 438 | AGACGCATGCGATGTATGGTAGG |
| 439 | ACAGGCCCCGGTAGTCACTTCGG |
| 440 | TACTGAACCGCAATAAACAAAGG |
| 441 | ACATCATGTGCGTAACATTGTGG |
| 442 | CTAAGTATATCATATTGACCAGG |
| 443 | TAGGGTCTTAAGCACCACAAAGG |
| 444 | AGAACCCCTCCCAACACGCGTGG |
| 445 | CGTAATAAAAAGTTACCGCAAGG |
| 446 | AGTGCCGCCAGAGCTTACCAAGG |
| 447 | GTCGGCTCTGTCTCTACCGAGGG |
| 448 | GCAAGAGTAAACTTCCATAGAGG |
| 449 | TGTGGAACATCGGTGGGTGAGGG |
| 450 | AGGCCTGTCAAACTTCGTAAAGG |
| 451 | GCAATGGAGCAGGTCGTCAATGG |
| 452 | TTTTAGGGGAAGTTGTGCTAAGG |
| 453 | GGATCTATCATCAGGGCCGAAGG |
| 454 | GCGTCTGAACCAAAGATGTACGG |
| 455 | GTTACGCACATGATGTATAGAGG |
| 456 | CTACCCCTTTAACCGGCACGGGG |
| 457 | TATTGCGTGGAGTAAGCTGGTGG |
| 458 | GTCGGCAATATAAGTGAACGTGG |
| 459 | CTAACCTATAACCTCAGCATAGG |
| 460 | ATCCGGTCATATAATCATCTAGG |
| 461 | AGTGATCGTTTCCCCAAGTAGGG |
| 462 | GTAGGCGCTCCAGTGGCTCGGGG |
| 463 | GTTGATGACCCACCATAGATGGG |
| 464 | GTTAGGGGGATGGCCGATGTTGG |
| 465 | TATACAACCGTTCCTCTCAAAGG |
| 466 | ACGATTAACCTCTAACTGCCAGG |
| 467 | GAAAACCTAACAACCAAGCTTGG |
| 468 | TGATGGTGCTGCAAAGCGACAGG |
| 469 | GTTCACACCTAAACCGGGAGTGG |
| 470 | GATGAAACTGTTCCACCGGCCGG |
| 471 | TCGAGTCAAACCCAGTTAGCCGG |
| 472 | GATAACCTATAGTCAGGGCATGG |
| 473 | TACGCCGGCTGAACGTGAGAAGG |
| 474 | GACACGCAAAGTCAGCTACATGG |
| 475 | GTGATTTCGAGGATGTCGCTGGG |
| 476 | ATACCACCTGTGTAAACTGCAGG |
| 477 | GTGTTCAGTAACACGTTGCAAGG |
| 478 | GTCCTATTCTCATTAACCTACGG |
| 479 | TGCTTCACTACTTGATCTGAGGG |
| 480 | TGGTATGCCCCTATGGATCAAGG |
| 481 | CTGTAGTGCACTTTGAGCGAGGG |
| 482 | ATACACGTGATCTGGCCCTAAGG |
| 483 | TACTTATTGACCTACTAAGCTGG |
| 484 | CGTTTAGGCATAGTAGAGACAGG |
| 485 | CAACAGGTAGGGGTCATAGAAGG |

TABLE 12
Target sequences for BRCA1 gene

| SEQ ID NOS | Target sequence |
|---|---|
| 486 | TCAGGTAGCACTCTTAACCTGGG |
| 487 | CCTGTCACCTGTCTATGGGTCGG |
| 488 | ACATAGACCCCTCTGTTGATGGG |
| 489 | GTAGTCAGACTAGTTCAATGAGG |
| 490 | GTAGTTGACCTGCACTCTACAGG |
| 491 | GTTTGATGTTTATCCAGACTTGG |
| 492 | TACTCCACTATGTAAGACAAAGG |
| 493 | GCTTTAACTTGTTAGATGCAAGG |
| 494 | AGTGCTAGATACTTTCACACAGG |
| 495 | TGTAATTTGGATTCCCGTCTCGG |
| 496 | ACGTCATATTTAAGGCATTCAGG |
| 497 | GCTAAGATCTGAACCCGAGACGG |
| 498 | GTATCTGAAGAACCGTTACCCGG |
| 499 | TTCCAAATATCCATACCTGCTGG |
| 500 | GTATCTTTACCATCTACCTCTGG |
| 501 | CTTTCAGGCAATCACTCCATTGG |
| 502 | GTCAACCCTGACATATTGGCAGG |
| 503 | ATATGTCAACCCTGACATATTGG |
| 504 | GCTGCAGATTAGACTACAAGTGG |
| 505 | CCATCGCCACCAATTGTGAAAGG |
| 506 | GTCAGGGTTGACATATAACATGG |
| 507 | TTATGCTAAGTAACTACCTATGG |
| 508 | CACGTAGAGGTTAGATGTGATGG |
| 509 | AACTACTCACGAGTACCACGTGG |
| 510 | TTTATGTAATGGCCACGTAGAGG |
| 511 | GTATTTGGCCACTTACCTGCTGG |
| 512 | TACTCACGAGTACCACGTGGTGG |
| 513 | TATTTGGCCACTTACCTGCTGGG |
| 514 | GGTTGCCAAAAAGTCCAGTGGGG |
| 515 | CATCACATCTAACCTCTACGTGG |
| 516 | TGGTTGCCAAAAAGTCCAGTGGG |
| 517 | GGTAAGTGGCCAAATACATTAGG |
| 518 | GTTGCCAAAAAGTCCAGTGGGGG |
| 519 | GCAATGCCATTGCCACCACGTGG |
| 520 | ATTACTGGTGGACTTACTTCTGG |
| 521 | AAGTAAGTCCACCAGTAATTAGG |
| 522 | GCTTTGCTACAATCCAATTCTGG |
| 523 | GGTACTTGAAGCATCTATATCGG |
| 524 | TAGAAAGTAGCCCAGCGCAATGG |
| 525 | AAGGTACTTATGTACAGTGGAGG |
| 526 | GGTTATATTGGATCCAGAATTGG |
| 527 | GTAGAGTGGTTAGCCCAAGGTGG |
| 528 | ATGTTGGTACAAGTTATCTCAGG |
| 529 | GAGATAACTTGTACCAACATTGG |
| 530 | AACTACGAGTGCGCAGACATGGG |
| 531 | CTCGGTCCCTCAGAACACGAAGG |
| 532 | AAAACGACCACCCCATTGACTGG |

TABLE 13
Target sequences for BRCA2 gene

| SEQ ID NOS | Target sequence |
|---|---|
| 533 | CACGTAAGCACTCTCCCACC |
| 534 | GTACTTTACCATCATGCAAG |
| 535 | AGGCCCCTGATTTACACTCT |
| 536 | GTCACTGGTTAAAACTAAGG |
| 537 | TCACTGGTTAAAACTAAGGT |
| 538 | GTCACAAATTTGTCTGTCAC |
| 539 | CCTTAGTACTACTCACAAGG |
| 540 | CTTCCTTAGTACTACTCACA |
| 541 | CCACCTTGTGAGTAGTACTA |
| 542 | GGTGTCTCTCTGTAATACAT |
| 543 | CGGTGTCTCTCTGTAATACA |
| 544 | GCAAATAACACCTCCCATGA |
| 545 | CTCTCAAAGATGGCACGTAC |
| 546 | ATATACTACCCATTAATGGC |
| 547 | AATCCAGTTCATTAAACCCC |
| 548 | ACACTTTGGGTTAGATATCC |
| 549 | AACTTCCCCTCATCTACTTG |
| 550 | ACTTCCCCTCATCTACTTGA |
| 551 | GGGACCCTCAAGTAGATGAG |
| 552 | TTGGGACCCTCAAGTAGATG |
| 553 | TGGGACCCTCAAGTAGATGA |
| 554 | AGGATTATCAAGTACACTCC |
| 555 | GATGTGCCAGACGAGTGTGG |
| 556 | TTGATAATCCTCTACCCTAA |
| 557 | CTTGATAATCCTCTACCCTA |
| 558 | TAATCCTCCACCCACACATA |
| 559 | TAGAGCCTGCCCATATGTGT |
| 560 | CAGCAAGCTGTCATATGATC |
| 561 | TCTTGGAACAGACGTGAGGT |
| 562 | TAATTCTTGGAACAGACGTG |
| 563 | AATTAGGCACCCCAGGATAT |
| 564 | TGCAAGAAATTAGGCACCCC |

TABLE 14
Target sequences for C9orf72 gene

| SEQ ID NOS | Target sequence |
|---|---|
| 565 | ACACTCCGATGATTATCCACTGG |
| 566 | TCTAACTCATCGGGGTCAAGTGG |
| 567 | TAGACAAGATCCCTATCCCATGG |
| 568 | TACTCTCAACTAAAAGTTAGAGG |
| 569 | CAGTAACAGCAAGGTGAGTCAGG |
| 570 | ACATGCAATGAGGTAGTGACTGG |
| 571 | TGTTACTGACGTGGTATCACCGG |
| 572 | GTTACTGACGTGGTATCACCGGG |
| 573 | CGTGGTATCACCGGGAATCATGG |
| 574 | GAATGTCCGCGCTCCACAGATGG |

TABLE 15
Target sequences for CATSPER2 gene

| SEQ ID NOS | Target sequence |
|---|---|
| 575 | GAGACTTCCGGTCCAGAAAC |
| 576 | ATTGCCAGGTGAGCTTGACT |
| 577 | CATAACTCTCATGTCAGATG |
| 578 | TGTCAGATGTGGGCCAAACT |
| 579 | GGGATGTCTAGTCTGTAGAC |

TABLE 16
Target sequences for CDH1 gene

| SEQ ID NOS | Target sequence |
|---|---|
| 580 | TAGGTTTGGGTGAACTCTAA |
| 581 | CCCATTTACAATCAACCTTA |
| 582 | GTCCAATCTGCCGTAACCTC |
| 583 | TCCAATCTGCCGTAACCTCA |
| 584 | GCCTGGTGCTAACACACAAC |
| 585 | GGACGGAACATACATGCCAA |
| 586 | GACGGAACATACATGCCAAT |
| 587 | ACATGAATAAATCGCTATCT |
| 588 | CCCTTAAGGTTGATTGTAAA |
| 589 | ACCCCAGGTACATGAGTCAA |

TABLE 17
Target sequences for CDK4 gene

| SEQ ID NOS | Target sequence |
|---|---|
| 590 | AATCTCTAGGGTACTTCCGG |
| 591 | CTAACCTTTGGGAGTGCCTA |
| 592 | CAAGTCCTCTTGTATGGCCT |
| 593 | gattaagggggtttgtctga |
| 594 | TCTAACCTTTGGGAGTGCCT |
| 595 | GGGAATGAACTGAAGGCCGT |
| 596 | AAGAGCTGTGCAAGTGTCGG |
| 597 | TGCAAGTGTCGGAGGTGTGA |
| 598 | AAGTTGACTAGGTGTGTGTC |
| 599 | GTGCAAGTGTCGGAGGTGTG |
| 600 | TAAATGACGCAGGTGTACCA |
| 601 | GGTGAGTGGTTAAATGACGC |

TABLE 18
Target sequences for CDKN2A gene

| SEQ ID NOS | Target sequence |
|---|---|
| 602 | CAGTTAGGAAGGTTGTATCGCGG |
| 603 | CAAGATATACTGGGTCTACAAGG |
| 604 | GCTAATTGAGAGGTACCCCGAGG |
| 605 | GGTGATTTCGATTCTCGGTGGGG |
| 606 | AGGTGATTTCGATTCTCGGTGGG |
| 607 | CAGGTGATTTCGATTCTCGGTGG |
| 608 | GTACAGGTGATTTCGATTCTCGG |
| 609 | AGCCGTTTTACACGCAGGAGGGG |
| 610 | CAGCCGTTTTACACGCAGGAGGG |
| 611 | GGCTTAACACAGCTGTACCTGGG |

TABLE 19
Target sequences for CDKN2B gene

| SEQ ID NOS | Target sequence |
|---|---|
| 612 | ATCTGAGTTATGTGCAACATTGG |
| 613 | TATCAGACGCTGCTTGTCAGGGG |
| 614 | TGCGGCAATTGACAGCATAGGGG |
| 615 | TTGCGGCAATTGACAGCATAGGG |
| 616 | CTAGTAAGCGCGAATGCCCCCGG |
| 617 | GCTCAAACTAAAGCGCCGCCGGG |
| 618 | CTCAAACTAAAGCGCCGCCGGGG |
| 619 | TCGCTTCATGGTGAGTGTCGAGG |
| 620 | CGCTTCATGGTGAGTGTCGAGGG |
| 621 | GGCTTTCCGCCGCTCCCCGTTGG |

TABLE 20
Target sequences for CTFR gene

| SEQ ID NOS | Target sequence |
|---|---|
| 622 | GTATGCTTTTGCCCACGGAA |
| 623 | GACCCTTGCCTTAGATGTGT |
| 624 | TTGCCGACACATCTAAGGCA |
| 625 | CTTTATTGCCGACACATCTA |
| 626 | GTGTCGGCAATAAAGTAATC |
| 627 | TGCCGACACATCTAAGGCAA |
| 628 | CACAATAAGGCCAAACAAGT |
| 629 | GGTCACACTATGCCACAATA |
| 630 | ATGTAGAGTGCCCACTTGTT |
| 631 | TCCAAGCACCCTAGACTGTA |
| 632 | GATAGAATAGAGCACACCAT |
| 633 | AAAGTGATGGCACACCCACC |
| 634 | TGGGATAGTATGCACCAGGT |
| 635 | ATACTGGGATAGTATGCACC |
| 636 | CTGAAGACCTTGCATGATCA |
| 637 | AAACACGCTTTCCCCTTCAA |
| 638 | GGATAATTAATACGCCATGA |
| 639 | GGAACTAGCAGCACCTTTGA |
| 640 | CAACTCCTACTGATAACCAA |
| 641 | ATTGGTGAGTAAAGGATCCT |
| 642 | TATTGGTGAGTAAAGGATCC |
| 643 | CATGGAGCTGTTACCATTCA |
| 644 | GACTATGTCCTCTTCGGTTG |
| 645 | CAGTACTCTATTGTCCCTAG |
| 646 | CCATTGTAGGCCAATAAGTG |
| 647 | GGAGGGTTGTCCAACCACTA |
| 648 | AATCACGATCTCTAAACTGG |
| 649 | ATCACGATCTCTAAACTGGA |
| 650 | CGGGTGTAGAGATCAAATAA |
| 651 | GTAGAGATCAAATAAGGGGC |
| 652 | TGGAGGGTTGTCCAACCACT |
| 653 | TGCCTTAATCCAACATTGGA |
| 654 | AATGTGCCTTAATCCAACAT |
| 655 | TTGGATTAAGGCACATTAGT |
| 656 | TGCCAGGTTAAGTTGTTCTT |
| 657 | GCCAGGTTAAGTTGTTCTTA |
| 658 | ACCCTAAGAACAACTTAACC |
| 659 | TGTATTAGCAAGTGGACTCC |
| 660 | GGGTCAATTGTATTAGCAAG |
| 661 | ACACTAACACCTACCCTACC |
| 662 | AGATCCTGAACTGTCTAGCC |
| 663 | CAGCCTACAAGTTCTTTGAC |
| 664 | CTGAGCTAGAGGTACCCTTA |
| 665 | GTAATTTAGATCTTAGGACC |
| 666 | AGACTTACTTACCAGGGAGC |
| 667 | CATGTACATTGGACCCTAAC |
| 668 | GGACCCTAACAGGAGTTCCA |
| 669 | TGTCTTAGATGATTCTAGTC |
| 670 | GAGTTTGGGGGCACACGAAA |
| 671 | GCTATTACTAAAGGTTTCTC |
| 672 | ATGGCCTTCAAAGTTGGCTC |
| 673 | CCCTTGAATAAGAGATATCC |
| 674 | ATGGCCTACACGACCCTACA |
| 675 | GGTCGTGTAGGCCATCTTAA |
| 676 | GTAGACAGCACGATGATTTC |

TABLE 21
Target sequences for CHEK2 gene

| SEQ ID NOS | Target sequence |
|---|---|
| 677 | AACGCACTGAGCTGTGTAGGAGG |
| 678 | TATTACTTGCAAGCTGAAACAGG |
| 679 | GTCATATGGGGAACTTCTGTTGG |
| 680 | ACCTCGACGTGTCTCTCGCCCGG |
| 681 | TGTTGACACAATACTTCAGCAGG |
| 682 | TTGGCAAATCGTATCTATGCAGG |
| 683 | CAACGTATGTATGTAGTAGTGGG |
| 684 | TCAACGTATGTATGTAGTAGTGG |
| 685 | GCAGATGTTCTAAGCTCTTGTGG |
| 686 | AAGGTGACCCTTATTAAAGTAGG |

TABLE 22
Target sequences for CLN3 gene

| SEQ ID NOS | Target sequence |
|---|---|
| 687 | GGGCATCGATTAGGGGTACGAGG |
| 688 | GCCAGAAGGGGCATCGATTAGGG |
| 689 | TGGGCGCCCCCCATCAGCTCAGG |
| 690 | CTTTCTCGTGCGGTTTTCCCAGG |
| 691 | GCTGTGAGGAGCTTCTCGAGAGG |
| 692 | AGTCCGACGAAAAGAGGGCCGGG |
| 693 | GAGTCCGACGAAAAGAGGGCCGG |
| 694 | GCATCATGCCAGGGTGCGCGAGG |
| 695 | GTTATCCCCGCCCAGTTCTGAGG |
| 696 | CTCCGCTTCTCTTCGGGTAAGGG |

TABLE 23
Target sequences for CLN6 gene

| SEQ ID NOS | Target sequence |
|---|---|
| 697 | CATGCACTCCTCAACTGTCGTGG |
| 698 | ACCCTTGAGATACGATCTACTGG |
| 699 | TTTGGTACGACCTGGATGAAGGG |
| 700 | TTTTGGTACGACCTGGATGAAGG |
| 701 | AGGAAGTTTTTGGTACGACCTGG |
| 702 | GAGTGAGCGGTCATCTTGGAGGG |
| 703 | GGTACACACACCTCGTCACTCGG |
| 704 | TTCCCGCGTTCCAGCGACCCGGG |
| 705 | CTTCCCGCGTTCCAGCGACCCGG |
| 706 | GAGCGCCCGCCCGAAGTTTGGGG |

TABLE 24
Target sequences for CNBP gene

| SEQ ID NOS | Target sequence |
|---|---|
| 707 | TTAATAGGGAGGGTAGTTCCAGG |
| 708 | TGGGGTGTTCGATGATTCAAAGG |
| 709 | GTTGGCAGGTATTGCTCAACTGG |
| 710 | GCTTCAGAAGCAAATACGAGAGG |
| 711 | AGTACTTGATTAGATTGGATTGG |
| 712 | CAGTGCAGTATACGTACATCAGG |
| 713 | ACCACCTGATTCACTGCGATAGG |
| 714 | GTTCCCACATGTTAACCATATGG |
| 715 | ATGCGGGTCTTTCGGCGCCACGG |
| 716 | AGCTGGGTCGCCGAGCATGCGGG |

TABLE 25
Target sequences for COL3A1 gene

| SEQ ID NOS | Target sequence |
|---|---|
| 717 | TTGGTGTGAAGAGTAATAACAGG |
| 718 | TCTATACTGCAGGTAAAGCAAGG |
| 719 | CTCTCAACTATGATACTTACAGG |
| 720 | GACTACCATTAATCCCAGGAGGG |
| 721 | GGACTACCATTAATCCCAGGAGG |
| 722 | AAACTTACGCGTTCACCACGTGG |
| 723 | TATTATGTCATCGCAGAGAACGG |
| 724 | TACCGTAATTGTTATACCTGAGG |
| 725 | GAAACATTTGTACGTACAGCTGG |
| 726 | GCCAGTTTTAGG1AACAATGAGG |

TABLE 26
Target sequences for CRB1 gene

| SEQ ID NOS | Target sequence |
|---|---|
| 727 | CCTGGAAACGAACAGCACCAAGG |
| 728 | CAGTAACCTAACTTACAGGGTGG |
| 729 | GACTCAAAGATAGTGCCGGGAGG |
| 730 | TCATCAGTAGAGATCCTGGGAGG |
| 731 | GATAAGCTCTGGTAACAGGGTGG |
| 732 | GACATGTGTATTCTATACGGTGG |
| 733 | ACCTCCAGCATAACACAAGGAGG |
| 734 | TAGGGGCTAAAACCGACATGCGG |
| 735 | TGAATTGCAGGAACTCATCGCGG |
| 736 | CTATAAGTAGAACGTCTGGGAGG |

TABLE 27
Target sequences for CRX gene

| SEQ ID NOS | Target sequence |
|---|---|
| 737 | ACACATCTGTGGAGGGTCTTGGG |
| 738 | GGCGTAGGTCATGGCATAGGGGG |
| 739 | GGGGCGTAGGTCATGGCATAGGG |
| 740 | CGGGGCGTAGGTCATGGCATAGG |
| 741 | ATCCCGGGATCTAAACTGCAGGG |
| 742 | CATCCCGGGATCTAAACTGCAGG |
| 743 | GCGGTCACAATCGTGCCAGACGG |
| 744 | GAGCTCGTGGTGTACTTCAGCGG |
| 745 | CTTACCAGTTACTCACCATGGGG |
| 746 | CACTTACCAGTTACTCACCATGG |

TABLE 28
Target sequences for CTNS gene

| SEQ ID NOS | Target sequence |
|---|---|
| 747 | GATGCCACGTACAGTTACCGAGG |
| 748 | GGTCTTAGAAAACCATCGTGGGG |
| 749 | TTATGCGCTTCCTTACACGAGGG |
| 750 | GAATCACAGGAGATCGCTAGCGG |
| 751 | ACGATCAGTCTCCAGCATGTGGG |
| 752 | ATTGGTTACTTACTTCATCGGGG |
| 753 | AGCGCAGAGGAGATTCACGATGG |
| 754 | AGAATGCTCACCCACGCAGGAGG |
| 755 | CTTGACGCCGCAATCCTCCAGGG |
| 756 | GCCGATGTTGAATACACTGTAGG |

TABLE 29
Target sequences for CYPC1 gene

| SEQ ID NOS | Target sequence |
|---|---|
| 757 | AATCTCTGATAGTATAAGATAGG |
| 758 | CTCATCATTAAACGTCACTACGG |
| 759 | GGGGGGGTCTCCCTACAGTAAGG |
| 760 | CATAAGTGTGGTGGTATCATGGG |
| 761 | GACTGTAACGCTTGTGCGATAGG |
| 762 | GTACCGAGGGTCAAAGATGGTGG |
| 763 | GCAATCAGGAAACCTCGTGTAGG |
| 764 | TGTCCAAACAAGTAACTACCAGG |
| 765 | CAAGTAAGCTCAGTGATCCAAGG |
| 766 | ACACCGATCTTTATCCCCCTGGG |
| 767 | GACACCGATCTTTATCCCCCTGG |
| 768 | TCTCGCTATTGAAACATTGTTGG |
| 769 | TACTCTTATACCCCAAAGTGAGG |
| 770 | GAGGGTCCAGATCAATCCATTGG |
| 771 | TGAACATTCGACCTCCATTACGG |
| 772 | GCAAGAGGCATAATGTGGGCAGG |
| 773 | CAATTCTGAATCATGACAACAGG |
| 774 | GATTCAATGGGTAATCCCCTTGG |
| 775 | TTAGTTATACTCTACACATAAGG |
| 776 | GTCCTGAGTACTTGGATACTTGG |
| 777 | GTACTGCCCTTCTTTGGAACGGG |

TABLE 30
Target sequences for CYP2C19 gene

| SEQ ID NOS | Target sequence |
|---|---|
| 778 | GGAGAACTATTAGTCATTGCTGG |
| 779 | TAGTAGGCTATATTAAATAGAGG |
| 780 | GTTAAGGGTCATCACTTTCAGGG |
| 781 | CTAACGTTTAAATCTTTGGCCGG |
| 782 | AGTATTGTAATCTATATGGGAGG |
| 783 | GTCCCCTCAATATTAGTATTTGG |
| 784 | GGGGCGCACGCATGTGTGACAGG |
| 785 | TTGTTCTGGCTACTCTTAAGTGG |
| 786 | GTACTAAATCAGTGACCTCAGGG |
| 787 | CTTATGTCAAGGGAATCCACTGG |
| 788 | AACTCCTCACTCACCTCTATAGG |
| 789 | TTGCTAAAATGCCCACAATCAGG |
| 790 | ACTGTTCGGTGAATCATAGGAGG |
| 791 | AGAACTGTTCGGTGAATCATAGG |
| 792 | CTACATATACTGCAGTATTGAGG |
| 793 | TGAATATCCCAATATAGATCAGG |
| 794 | GCGAATATAATACGTTTTTGTGG |
| 795 | CTTTAGTCTGGTGGCCACATTGG |
| 796 | AATAGACCTGCTGAATATGTTGG |
| 797 | AATGGCCCTATCACACCCCTAGG |
| 798 | ACGAGGAGTATGTACAAGGGAGG |
| 799 | CAGGTTTGTCATCGTACCCCAGG |
| 800 | TTATCCGATTTTACAGTGTGTGG |
| 801 | AAAGGAGCACCGGGCTGTATGGG |
| 802 | TAGTTACACCCCCATTGGAAGGG |
| 803 | ATAGTTACACCCCCATTGGAAGG |
| 804 | CTTTATAGTTACACCCCCATTGG |
| 805 | GGCTCCACTCCTCAATCTTAGGG |
| 806 | GGCTACTCACCCTTCAACTGGGG |

TABLE 31
Target sequences for CYP2D6 gene

| SEQ ID NOS | Target sequence |
|---|---|
| 807 | TCCGGTGTCGAAGTGGGGGGCGG |
| 808 | GAATCCGGTGTCGAAGTGGGGGG |
| 809 | CGGCCCGAAACCCAGGATCTGGG |
| 810 | GACGAGATCTCCAAATGCCCAGG |
| 811 | CCCTCTACAGGTGGATTGTATGG |
| 812 | GCCATACAATCCACCTGTAGAGG |
| 813 | CGGGGTTGATAAGTCCGCTGGGG |
| 814 | GGGGTTGATAAGTCCGCTGGGGG |
| 815 | ATAAGTCCGCTGGGGGTGACGGG |
| 816 | GGCACAGGATTCACTTATTGAGG |
| 817 | GCAGTCCGGTGGAGTGCTGTCGG |
| 818 | CTTTCCGACATACACGCAATGGG |
| 819 | AATTGTTCCAATCTGCTCTTGGG |
| 820 | CGGCTGGAACCTGCTGATCTCGG |
| 821 | GGGCGATAATGTGGCAACTCCGG |
| 822 | GAAGCGAAGTCTTTGCCGAGTGG |
| 823 | AAGCGAAGTCTTTGCCGAGTGGG |
| 824 | CCGGGCGTGGCTTCAGTGCTCGG |
| 825 | ACCTCCGGTTGCTTCCTGAGGGG |
| 826 | GTCAAGACAAGTTCTCACAGAGG |
| 827 | AAGGCGAGGTCGTTAAAGAAAGG |
| 828 | AGGCGAGGTCGTTAAAGAAAGGG |
| 829 | CAGCCTCGTCACCTCACCACAGG |
| 830 | ACGTACCCCTGTCTCAAATGCGG |
| 831 | GCCTGGCCGCATTTGAGACAGGG |
| 832 | TCGAAATCTCTGACGTGGATAGG |

TABLE 32
Target sequences for CYP11B1 gene

| SEQ ID NOS | Target sequence |
|---|---|
| 833 | CGCGTGTTCCTCTACTCTCTGGG |
| 834 | GCGCGTGTTCCTCTACTCTCTGG |
| 835 | ACAGTACACCAGCATCGTGGCGG |
| 836 | TCAACAGTACACCAGCATCGTGG |
| 837 | CATGACGTGATCCCTCTCGAAGG |
| 838 | CAAGGCTCTTGGATAAGATAAGG |
| 839 | GCGTACCAGATGACGAGAGTGGG |
| 840 | AGCGTACCAGATGACGAGAGTGG |
| 841 | GCTGCTAAACCGGGTCAGGTGGG |
| 842 | ATGGTGAGGAGCGTACCATCTGG |
| 843 | GAGCCGGTACTGGGAGAACCTGG |
| 844 | AACACGCGCACCAATGTCTGCGG |
| 845 | GACCCCCCGAGCTGCGACACTGG |
| 846 | ACGATGCTGGTGTACTGTTGAGG |
| 847 | TGACCCACAGGGCTTATCAGTGG |
| 848 | CAATGCAGGCACACCCCATTTGG |
| 849 | ACGCCGGGGCATGGCTTCAAAGG |
| 850 | CTTCGAGAGGGATCACGTCATGG |
| 851 | TTCGAGAGGGATCACGTCATGGG |
| 852 | GGGGGTGCATGAGCGTAGACAGG |
| 853 | GGATTATTCATCTCCTTGCAAGG |
| 854 | CTTAGAGATTTTCAAGTCCGTGG |
| 855 | TCATGCCCACTCTCGTCATCTGG |
| 856 | ACTCTCGTCATCTGGTACGCTGG |
| 857 | ATCACCAAATGTATTAGTGCAGG |
| 858 | GCACCGTTCCCCCTTGATACTGG |
| 859 | CTGTCAGTTCGAGGTGAATCTGG |
| 860 | AGTAGTGCATTCTGAACTGAGGG |
| 861 | GGTAAAAGGCTCTTTGGGGGAGG |
| 862 | GGTTATTAAGGATTGCCACAAGG |
| 863 | ACCGGTGAGTCATTCCAGTCTGG |
| 864 | CCGGTGAGTCATTCCAGTCTGGG |
| 865 | TGGTATATATGAGTGCTGTAGGG |
| 866 | GGCTGGGTACACTCTCAAACTGG |
| 867 | ATCCGGCCGGCCCAGAGTTCAGG |
| 868 | GTATGGCCACACGAGGAGCCTGG |
| 869 | TCGGGAGTTCCATTTGTGCTGGG |
| 870 | CGGGAGTTCCATTTGTGCTGGGG |
| 871 | GCAGAGACGTGATTAGTTGATGG |

TABLE 33
Target sequences for CYP11B2 gene

| SEQ ID NOS | Target sequence |
|---|---|
| 872 | TGTGAGAACCCGCCCTGAAGAGG |
| 873 | AACCGCCCTCAACACTACACAGG |
| 874 | CTTGTTGTAAGCGGCGAGTTGGG |
| 875 | TCTTGTTGTAAGCGGCGAGTTGG |
| 876 | ACGTATCGAGATTCCTCACATGG |
| 877 | CAGAAAAGCTCGTCTATGTCAGG |
| 878 | GGCTCTATGAATCTGAACTACGG |
| 879 | ACCTCTTCACTGCGTCAGCACGG |
| 880 | TGCGGCCAGACCTATGGGCAGGG |
| 881 | GTGCGGCCAGACCTATGGGCAGG |
| 882 | GGGGGGTGCGGCCAGACCTATGG |
| 883 | CCTTGGGCGACAGCACATCTGGG |
| 884 | ATCCCCACCTAAACACTGTCGGG |
| 885 | CCCCACCTAAACACTGTCGGGGG |
| 886 | CAGTGCAGACGCGACCCCACAGG |
| 887 | ACAGTAACCGCACCCCCGCCTGG |
| 888 | CCCAACCCGTGAACATTACAAGG |
| 889 | CCAACCCGTGAACATTACAAGGG |
| 890 | CTCACATGTGGCACGCTACACGG |
| 891 | GTACTTCCCGAATTACCAAATGG |
| 892 | CTGAGTTGAGGGCCGTTCTCAGG |
| 893 | ACCAGGCACCGAACCTTGCAGGG |
| 894 | AATCCCGAGATCTGCTCCGCTGG |
| 895 | AAGGCACTCACTCCAAGTTGAGG |
| 896 | AAGTTGAGGGGGGCGGCACCTGG |
| 897 | CAGAAAGGGCCGACCGCGGTGGG |
| 898 | CACCCCTCCGCATTCTCGTCAGG |
| 899 | ACCCCTCCGCATTCTCGTCAGGG |
| 900 | CAGAGCTTGCCGGCTAACTTGGG |
| 901 | AGAGCTTGCCGGCTAACTTGGGG |
| 902 | CGTTTCAGCGGGTGATTGCTCGG |
| 903 | TTTCAGCGGGTGATTGCTCGGGG |
| 904 | CCCGAGTCAAGTCCTTCCAACGG |
| 905 | CAACCAACATCCGCCCGCACAGG |
| 906 | TCCCGCTGTCATGTCAGAGCTGG |
| 907 | GCAACTGTCTTCGAATAGGCTGG |
| 908 | CAATAACATTGGCCAACCTCTGG |
| 909 | ATGGATCATACTGTTGTTCCAGG |
| 910 | ATTAACCATGGATTGTACCATGG |
| 911 | TTATGACCAAAAGGCCCCCATGG |
| 912 | TAACCACGCAACTTAGGCTCAGG |
| 913 | AGCTACACTAAGGCATGAACTGG |
| 914 | ACTTAGATGAAGGTGTTCGGGGG |
| 915 | TCCCGTGCCGAAGAGACACCTGG |
| 916 | GCCCAAGGCAGGTTCACGTAGGG |
| 917 | CATGGCTCCGTATCAACCAGAGG |
| 918 | TTACCAAAGTGTGACCTCGATGG |
| 919 | TAATCGCTCTGAAAGTGAGGAGG |
| 920 | TCTCCATGTATGAGCACTCCCGG |
| 921 | ACGCCGACCTCAACCAACCAAGG |
| 922 | CATTGCGACCCAGCGAGTAGAGG |
| 923 | GAGGTTACCGGTATGGAGCCAGG |
| 924 | CCTGTACCAATGTCTGCGGACGG |

TABLE 34
Target sequences for DMD gene

| SEQ ID NOS | Target sequence |
|---|---|
| 925 | CACTCATGCATCCTCTTAGATGG |
| 926 | GTGGTGGTTGACTATGGTAAGGG |
| 927 | AAATCCGAATCCCCAGGCCAGGG |
| 928 | GTGGCCAAATCCGAATCCCCAGG |
| 929 | TGAAACTCGCATTCATAAGGAGG |
| 930 | GCATTCATAAGGAGGCACACAGG |
| 931 | GTATATAGCAGTGCATGCCAGGG |
| 932 | ATTGTAATAGAGGAGGCCATAGG |
| 933 | TCTTGCCATATATGATCCTATGG |
| 934 | GTCCTCAGGAATACTGCCATTGG |
| 935 | AAGTACCATCTACACAGATCAGG |
| 936 | GCATCGAATCTCAAGAAATATGG |
| 937 | GATCTCAACATAACGTCTTCCGG |
| 938 | GGAAATGTAGTGAAGATCGGGGG |
| 939 | GGGAAATGTAGTGAAGATCGGGG |
| 940 | TGGCTAGCTTTCCCTACCAAAGG |
| 941 | GATCAAGTGCTTAATAGAGGTGG |
| 942 | TTGGTAGGGAAAGCTAGCCAGGG |
| 943 | AGGGTAAACAGGAACGCTTCAGG |
| 944 | ATGGATGGCCCTGAAGTCACAGG |
| 945 | GTATGGTGGGTCCTAAACAATGG |
| 946 | TATGGTGGGTCCTAAACAATGGG |
| 947 | CATACCTGCTACACGTATATAGG |
| 948 | GTCCAAATTGCTCTTTGAGCTGG |
| 949 | AATTCCTATATACGTGTAGCAGG |
| 950 | GGGATCTCAAGGATGTAGGCAGG |
| 951 | ATAGATTTCATGACGTACTAAGG |
| 952 | CCATAAGATTGCCTCAACTCAGG |
| 953 | GCCTCAACTCAGGTGTACCTCGG |
| 954 | CTGAGTTGAGGCAATCTTATGGG |
| 955 | TGAGTTGAGGCAATCTTATGGGG |
| 956 | ACGTCATGAAATCTATATAGTGG |
| 957 | CATTCCACTCAGGTACCTAAAGG |
| 958 | GATCTATCTGGCTTTCAATCAGG |
| 959 | TTTAAGGGGATCATTGCCACTGG |
| 960 | GTGCATACATACAAGTTCTATGG |
| 961 | TAGTAGGTGCTGGTATCACAAGG |
| 962 | GCTGTGGATTAGGCCTAGATTGG |
| 963 | CACGTCTTCTGACAATGAGATGG |
| 964 | ACGTCTTCTGACAATGAGATGGG |
| 965 | CGTGTTAAATATCCCTGTGTTGG |
| 966 | TAACACGTTGATTGCTGTTAAGG |
| 967 | TACTGCAAACCAGCCAACACAGG |
| 968 | ACTTGAATTGGAGCAATGCCTGG |
| 969 | GTTAAGGCTAAGATGTAGTTAGG |
| 970 | ATCTGGCTTTAGAGCTGAATGGG |
| 971 | TAACCACCACTCCTTCGTCACGG |
| 972 | GCACTATTTTGGTGGAATGCTGG |
| 973 | TAGTGGGATCACATCCCTGTGGG |
| 974 | TGGAAATTAGCCCGGTGGCATGG |
| 975 | TTACATGGAAATTAGCCCGGTGG |
| 976 | GAGATGGCATTACCCTTAGATGG |
| 977 | GAATGTTCTTGGAGAAGCGTTGG |
| 978 | AATGTTCTTGGAGAAGCGTTGGG |
| 979 | TCGGTGAGGTGAAAGATTAAAGG |
| 980 | GTGCATTTTAGAAATCGGTGAGG |
| 981 | ATGATACCCTTAAGGTACTTGGG |
| 982 | TCATAGACCCAAGTACCTTAAGG |
| 983 | CATAGACCCAAGTACCTTAAGGG |
| 984 | AACTATAGGTCCCACCCAACAGG |
| 985 | AGTTTGATGTGCTTTTCGAAAGG |
| 986 | ATGTGCTTTTCGAAAGGTTATGG |
| 987 | GTTCCTCAGAGCCTATGCCAGGG |
| 988 | TTAGGCCTCTTTCGGAGAGAAGG |
| 989 | AACAGTTTGTGTCGGTATAGAGG |
| 990 | AGATTTCAGGAGCCTAATAGAGG |
| 991 | GGCTATATTGTTGTCACAGCAGG |
| 992 | GAAGACCCAATCTTGACACCAGG |
| 993 | CTATACTGTGCCCTAAGATGAGG |
| 994 | CTGACCCTGGTGTCAAGATTGGG |

TABLE 35
Target sequences for DMPK gene

| SEQ ID NOS | Target sequence |
|---|---|
| 995 | GGGCACTCAGTCTTCCAACGGGG |
| 996 | CTGGTCATGGAGTATTACGTGGG |
| 997 | GATGGCGCGCTTCTACCTGGCGG |
| 998 | CGTCATTGGCTGCTTCCTAGCGG |
| 999 | GCGGTTGATCGACAAGACCAAGG |
| 1000 | TGGGCAGACGCCCTTCTACGCGG |
| 1001 | CAACTCCCCGAGTGGCACAGTGG |
| 1002 | ATAAATACCGAGGAATGTCGGGG |
| 1003 | GAAGTAACCTCGTCTCTCCGTGG |
| 1004 | AGTCCCCCACGTATATGGCAGGG |
| 1005 | CGAAGTTCTGGTTGTCCGTGCGG |
| 1006 | GACATTCTACATGAGAACGTGGG |
| 1007 | CCTTCTTATGAAACCCTTGGGGG |
| 1008 | CCCCTCTTCTCGACGCTCGGTGG |
| 1009 | GCCTGACGTAGTAAAGATCGGGG |
| 1010 | GGAGAGCGGTACCACTTGTGGGG |
| 1011 | GCTCCCGTTCACCAGGATGGAGG |
| 1012 | GTCTCAGTGCATCCAAAACGTGG |
| 1013 | AACCGCATCGTGAAGCAGGACGG |
| 1014 | TTGCGAACCAACGATAGGTGGGG |
| 1015 | GTGGGGTTCGCACTCTTACGAGG |
| 1016 | CAGCGTGCCCCCCTTTACACCGG |
| 1017 | GGACATTCTACATGAGAACGTGG |
| 1018 | GTCCTTCACCGAGGGCCGCGTGG |
| 1019 | TACATGGGAAGGTGGATCCGTGG |
| 1020 | TGCGAACCAACGATAGGTGGGGG |
| 1021 | CCAGGCCGTTGATGATGACGGGG |
| 1022 | GGGCCACACCCGTCACGATGGGG |
| 1023 | CAAATGCGCAGCTAAGCGGGTGG |
| 1024 | CCGGCCCACAACGCAAACCGCGG |
| 1025 | AAGAGGCATAGGGCGCGTGGAGG |
| 1026 | TCTAAAGTCGCAAAGACGTAGGG |
| 1027 | CCCAATAGAGGCTAAAACGGTGG |
| 1028 | GAAGCTCCCGTTCACCAGGATGG |
| 1029 | GTCATGGAGTATTACGTGGGCGG |
| 1030 | CACTTAGTCCCCGCGCCCCGCGG |
| 1031 | AGGTTCACGTTTCACAACAAAGG |
| 1032 | TCGAGCTTGCGTCCCAGGAGCGG |
| 1033 | GTCAACCTCACCCCCTGCGGTGG |
| 1034 | AAATATCCAAACCGCCGAAGCGG |
| 1035 | TAGGGTTCAGGGAGCGCGGGCGG |
| 1036 | ATGAAATGCGGGGTGTCGGAAGG |
| 1037 | GGCGCTTCTCGTCCGGCGTGGGG |
| 1038 | AAGATCCGCCCTCCTGCCGTGGG |
| 1039 | AAGCCTGACGTAGTAAAGATCGG |
| 1040 | AGCAAATTTCCCGAGTAAGCAGG |
| 1041 | CGGCCGGCCGCAGAGAGAAGTGG |
| 1042 | GCGAGGTCAACACCCGGCATGGG |
| 1043 | TGCGTCTTCAGCACCAATGTCGG |
| 1044 | GCAGCGGTTCAGAATCAAGCTGG |

TABLE 36
Target sequences for EGFR gene

| SEQ ID NOS | Target sequence |
|---|---|
| 1045 | GGGCACTCAGTCTTCCAACGGGG |
| 1046 | CTGGTCATGGAGTATTACGTGGG |
| 1047 | GATGGCGCGCTTCTACCTGGCGG |
| 1048 | CGTCATTGGCTGCTTCCTAGCGG |
| 1049 | GCGGTTGATCGACAAGACCAAGG |
| 1050 | TGGGCAGACGCCCTTCTACGCGG |
| 1051 | CAACTCCCCGAGTGGCACAGTGG |
| 1052 | ATAAATACCGAGGAATGTCGGGG |
| 1053 | GAAGTAACCTCGTCTCTCCGTGG |
| 1054 | AGTCCCCCACGTATATGGCAGGG |

TABLE 37
Target sequences for EPCAM gene

| SEQ ID NOS | Target sequence |
|---|---|
| 1055 | AGCAAATGATTCAACACCGGGGG |
| 1056 | AGGCTTTATATATGCCCCTCTGG |
| 1057 | GAGGTCTCTAAATCTATCAAAGG |
| 1058 | AACGGCAGCAGCGAACCATTTGG |
| 1059 | TCTAGCTGCCATCCCACTGAGGG |
| 1060 | TTAGGGTACTTGGGATACGAAGG |
| 1061 | CAGTCCCCCTCGCTACCCATTGG |
| 1062 | AAGATGAAGTTCTCCCGATTAGG |
| 1063 | AAAGATCCCTAACGCCGCCATGG |
| 1064 | CGCCATGGAGACGAAGCACCTGG |
| 1065 | CAACGAGCACCAGCGGCCAGAGG |
| 1066 | GCGAGCGAGCACCTTCGACGCGG |
| 1067 | CGAGCACCTTCGACGCGGTCCGG |
| 1068 | GAGCACCTTCGACGCGGTCCGGG |
| 1069 | AGCACCTTCGACGCGGTCCGGGG |
| 1070 | CCCCGCAGGTCCTCGCGTTCGGG |
| 1071 | GTTCGGGCTTCTGCTTGCCGCGG |
| 1072 | GCTTCTGCTTGCCGCGGCGACGG |
| 1073 | GCCCTCCGCGCGGTAGGAAACGG |
| 1074 | GTTTCCTGCGGCCACCGAACCGG |
| 1075 | CCCTGGCGCACCCACGTCCTCGG |
| 1076 | GCGCACCCACGTCCTCGGTTCGG |
| 1077 | GCACCCACGTCCTCGGTTCGGGG |
| 1078 | CCCACGTCCTCGGTTCGGGGTGG |
| 1079 | GGCCGCTATGCACCTGCGCGCGG |
| 1080 | GCTATGCACCTGCGCGCGGCAGG |
| 1081 | TATAATATTGCCCCAGCAGGTGG |
| 1082 | ATAATATTGCCCCAGCAGGTGGG |
| 1083 | TGTGTAATACTGATGTTCCCAGG |
| 1084 | GATCACAACGCGTTATCAACTGG |
| 1085 | ACAGTAGTAGGAAAGGCGTTGGG |
| 1086 | TGTTGATACAAGCTGTGCACAGG |
| 1087 | ATATTCTTGCGTGAGTTCCATGG |
| 1088 | CCATTCTGTAGTAGGTCATCTGG |

TABLE 38
Target sequences for ERG gene

| SEQ ID NOS | Target sequence |
|---|---|
| 1089 | CGGCACTGAATACATCCCAGAGG |
| 1090 | GTATTACATTGAGAACCATGTGG |
| 1091 | GGAATCTGACGATATCCCTGTGG |
| 1092 | AAAGCTGGTTCGATGCAGTGGGG |
| 1093 | ATCAGAGTCTACTTACAGCGAGG |
| 1094 | ATAACGTGATCACAGCGTGGCGG |
| 1095 | TTAATAACGTGATCACAGCGTGG |
| 1096 | GCTCACGAACACCATCACATGGG |
| 1097 | GATGCACAGAACACGCACAAGGG |
| 1098 | CGGGGCACAGGAGTACACCAAGG |

TABLE 39
Target sequences for EVC gene

| SEQ ID NOS | Target sequence |
|---|---|
| 1099 | TAGGTGGAAGATCTGAACCAGGG |
| 1100 | CCACCACACTCTCAATACGGAGG |
| 1101 | ATGCCTGAATAAACCCACCGGGG |
| 1102 | GCGATGCCCTGTGAGCAACACGG |
| 1103 | ATTTGAGAGATCCATCCGTGTGG |
| 1104 | GTGTCATCCCAATAACAGCGGGG |
| 1105 | TGTGGCTTAGATACCCTGGTAGG |
| 1106 | GCGCCCAAACCGAATCAGAGCGG |
| 1107 | GTGATGTGAGATCGTCAGGGAGG |
| 1108 | AGAGCGAAACCAGAGCTCGGTGG |
| 1109 | ATAATACAAGCATACCATGGAGG |

TABLE 40
Target sequences for EVC2 gene

| SEQ ID NOS | Target sequence |
|---|---|
| 1110 | GTATAGAAGACGAACCCCAGAGG |
| 1111 | ACCTACAATGTACCGCACAGTGG |
| 1112 | CGTAAGTGAACCCACCACAGGGG |
| 1113 | GCCGAAGCGTTAGTGCACAGTGG |
| 1114 | GGCGTAATCAGCAAACAGCGGGG |
| 1115 | ACAGGCTATATAGTCCAGAGGGG |
| 1116 | CCACCACACTCTCAATACGGAGG |
| 1117 | ATTGCGAAAGAATGGCCCAGAGG |
| 1118 | TAATATCTTTGAGTGCTACGGGG |
| 1119 | GCGCCCAAACCGAATCAGAGCGG |

TABLE 41
Target sequences for F8 gene

| SEQ ID NOS | Target sequence |
|---|---|
| 1120 | ACTGTAGTAAGAACACAACGTGG |
| 1121 | GTACACAGAATGACGCCACGAGG |
| 1122 | GTTGTGGGAGTGGAACTACGTGG |
| 1123 | TTATGGGCAGACAACCACACAGG |
| 1124 | CCGATCTGAGATACCCATGAAGG |
| 1125 | TACGATGGTAGACACAAAGGAGG |
| 1126 | GGACACACCCCACTAAACGATGG |
| 1127 | TGTATCGAGCAATAATTGGAGGG |
| 1128 | TGTATGCACTACTTCTGGAGGGG |
| 1129 | TGTTACGATGGTAGACACAAAGG |

TABLE 42
Target sequences for FBN1 gene

| SEQ ID NOS | Target sequence |
|---|---|
| 1130 | GAAGTCCAAGTACTACACAGTGG |
| 1131 | ATAACAGAGTGATACCCACGAGG |
| 1132 | CATATGTTTAGTCCACATGGGGG |
| 1133 | ACACTCGTCATTCAGCACCAGGG |
| 1134 | GGTACATACAAACACCTCTGGGG |
| 1135 | TGCTCATACGAAGACAACCGAGG |
| 1136 | GAGTGTATCAGATCACCTAGAGG |
| 1137 | TTTCTCCTTACCGATACACGCGG |
| 1138 | TACCAATACACTCCCCACGGAGG |
| 1139 | ACATACCATCAGGTTCCGTGGGG |

TABLE 43
Target sequences for FGFRI gene

| SEQ ID NOS | Target sequence |
|---|---|
| 1140 | CATGTGTTAACAGTGCATTGCGG |
| 1141 | GAACACGCTTGATACACATGTGG |
| 1142 | TACTGATCCAACATACAGGGTGG |
| 1143 | CTAAATTACAGTGACGAGGTGGG |
| 1144 | TGAGGAATGATCCCATTCGGGGG |
| 1145 | GTTGCCCGCCAACAAAACAGTGG |
| 1146 | GCACTGTCAAGGCTACGTGGGGG |
| 1147 | GTGAGGAATGATCCCATTCGGGG |
| 1148 | CCTCGACGTCCATCCAACTGAGG |
| 1149 | ATGAGTCCAGAAGTTGCGGGGGG |

TABLE 44
Target sequences for FGFR2 gene

| SEQ ID NOS | Target sequence |
|---|---|
| 1150 | TGACCAAACGTATCCCCCTGCGG |
| 1151 | GTGCGTTGCTTGGATCAATGGGG |
| 1152 | CAACTGTTACCTCCCACCCGGGG |
| 1153 | AACCAGTGCACTAAACACGTGGG |
| 1154 | TCCAGGAGTACTATCCACCTGGG |
| 1155 | AGACCAATGAGATTCCACGTGGG |
| 1156 | GTTGCGTTGACGTAATGACAGGG |
| 1157 | ACTTTAAAGTCCCCGCCATGTGG |
| 1158 | ATGACGTTAACACCCAGCAGAGG |
| 1159 | GAGGCCCTTAGAGCGTTCCGAGG |

TABLE 45
Target sequences for FGFR3 gene

| SEQ ID NOS | Target sequence |
|---|---|
| 1160 | ATCGTGAACGTATTGCCAAGTGG |
| 1161 | GAATTGCCGCTCACACCACAGGG |
| 1162 | GAGATCGCATGGCTCCCAGGGGG |
| 1163 | TTTCCGTCATGACCGCCGTGTGG |
| 1164 | AGAAGCTCCGTACCCCCGGGAGG |
| 1165 | CATCGTGGCACAGACATGGGGGG |
| 1166 | GACCCCCAAGGTACAGATCGAGG |
| 1167 | GTTAGAATATACCTCGTGTGAGG |
| 1168 | GTGCGTAGTGGGCAGAACGGCGG |
| 1169 | CGTGCAGGTGAGGGTCATCGTGG |

TABLE 46
Target sequences for FMR1 gene

| SEQ ID NOS | Target sequence |
|---|---|
| 1170 | TACACTAACCATCATAGTAG |
| 1171 | GGCATACTCGGTAGCAAACTAGG |
| 1172 | AACAATCTGCTATCAGTAAC |
| 1173 | CTGGGTTTGAGCACATCAAT |
| 1174 | GTATGTTTGCAATACAACACTGG |
| 1175 | AAACTGCTGGAGTACCCCAA |
| 1176 | AAGAGGACTATAACGGCAAG |
| 1177 | GCTTAAATTAGAGTGGCCCTTGG |
| 1178 | GATTGGATATGTCTCATTGCCGG |
| 1179 | CTTAAATTAGAGTGGCCCTTGGG |
| 1180 | TGCCAGACTTGGAGTGCCAAAGG |
| 1181 | CAACTATTCTAATGGCACTTAGG |
| 1182 | GACTGCATCAACTATTCTAA |
| 1183 | TAATGGCACTTAGGTGCTGAGGG |

TABLE 47
Target sequences for FXN gene

| SEQ ID NOS | Target sequence |
|---|---|
| 1184 | GTAATCCAGATACACCCAAGAGG |
| 1185 | GGCCTAAAGTAAGACACCAGGGG |
| 1186 | CTGCTGTAAACCCATACCGGCGG |
| 1187 | ACTTAGGGCAAGGTTACACAGGG |
| 1188 | TATCAGAGTATAGGGCCAAGGGG |
| 1189 | TACCCTGAGAGGATCGCATGTGG |
| 1190 | TATCTGACCCAGTTACGCCACGG |
| 1191 | TTTCAGAGTTCGAACCAACGTGG |
| 1192 | GGGCCTAAAGTAAGACACCAGGG |
| 1193 | GAAGTCAAGAGGTACCCCAAAGG |

TABLE 48
Target sequences for G6PD gene

| SEQ ID NOS | Target sequence |
|---|---|
| 1194 | GGTTCTGCATCACGTCCCTGGGG |
| 1195 | GCCGTGAGTTGATGTGACATGGG |
| 1196 | GGGGATTCGGGAGCACTACGCGG |
| 1197 | CAATGACAATATGCGTGGAGCGG |
| 1198 | AAAAAACCCGGTAAATTGCGGGG |
| 1199 | GUTTTTGAAACGAGGGCCCAGGG |
| 1200 | ATAATGGGAGAGGATTGCGAGGG |
| 1201 | GCTTCATCTCAAATTACACGTGG |
| 1202 | CTCGGTAATGATAAGCACGCCGG |
| 1203 | AGTAGGCGCCCAGAGCTGAAGGG |

TABLE 49
Target sequences for GAA gene

| SEQ ID NOS | Target sequence |
|---|---|
| 1204 | AATAGCAACGAGACCTGAGGGGG |
| 1205 | AGTTGGCATCAGTTCCAACGAGG |
| 1206 | CCGCAGGCTGAACACGACGGTGG |
| 1207 | ACCGTCCCCACTCTACAGCGTGG |
| 1208 | CTTAACGCACGCCAGAAACGCGG |
| 1209 | TCCAGCTAACAGGCGCTACGAGG |
| 1210 | TTGATTATATTCTCTCACGTGGG |
| 1211 | CTCCTTGATAACCTACACTGCGG |
| 1212 | GGTCGTACCATGTGCCCAAGGGG |
| 1213 | GCGGTCATTATAAATCTGCGTGG |
| 1214 | AACGCGGTGCTGCTTCAACACGG |

TABLE 50
Target sequences for GALC gene

| SEQ ID NOS | Target sequence |
|---|---|
| 1215 | TACTATACACCCACAATTAAGGG |
| 1216 | ACGCCGCTTTCATGATGTTCTGG |
| 1217 | ATGGGGCGCTGTTTTCTATCAGG |
| 1218 | TACTACTCAAACCACTCCTAAGG |
| 1219 | AATACGAATGCTGGTCTGTCTGG |
| 1220 | ATGTATGGCCCACTACTTAGTGG |
| 1221 | GTCTTGGAAGTATAACGTAATGG |
| 1222 | CCTCCCTGGTTAGAGAATCAGGG |
| 1223 | TACAGAGTATATGGGTCTTGTGG |

TABLE 51
Target sequences for GALT gene

| SEQ ID NOS | Target sequence |
|---|---|
| 1224 | GAATGAGCTCAATACCCCCGAGG |
| 1225 | AGGCAGACCTTATCACCCTGGGG |
| 1226 | GCTTGTATCAACATTCCCCAAGG |
| 1227 | GATCCGCTGGAAAATCTGCAGGG |
| 1228 | GAAGTCGTTGTCAAACAGGAAGG |
| 1229 | TGGGGATTCACCTACCGACAAGG |
| 1230 | ATGTCTGCCAGCGTGAGAGTGGG |
| 1231 | gtataagcgctcgtgacagaggg |
| 1232 | CTAGGCAGACCTTATCACCCTGG |
| 1233 | GACAATTCACTAAGAACCCTGGG |

TABLE 52
Target sequences for GATA6 gene

| SEQ ID NOS | Target sequence |
|---|---|
| 1234 | GCCGAAATAAATCAACCCTGGGG |
| 1235 | TTTTTLLGCGAAGTGCACGGGGG |
| 1236 | GCCAATATAGGAGAACGCGGCGG |
| 1237 | ACCCGAGTTAAAGTTCCCAAAGG |
| 1238 | CCGGGGGAGACACTTTAGGGCGG |
| 1239 | TACTCCAAACAGTCCTACCCCGG |
| 1240 | CTlTTTATTCACCAGCAGCGCGG |
| 1241 | CTTATTGATCTCCACGCCCGGGG |
| 1242 | TCGAATCGCGAATAGTGGTGTGG |
| 1243 | TCGCGAATAGTGGTGTGGCGCGG |

TABLE 53
Target sequences for GBA Gene Cluster

| SEQ ID NOS | Target sequence |
|---|---|
| 1244 | AAGCCATGGACGTTAGTAGT |
| 1245 | TAGAAAAGAGGGCTTACGGT |
| 1246 | AGCCATGGACGTTAGTAGTA |
| 1247 | AGGGCTTACGGTGGGCAATG |
| 1248 | AAAGATGGTACTTAAAGCCA |
| 1249 | GTAGAAAAGAGGGCTTACGG |
| 1250 | ACCAGATATGCTGAGTTGGA |
| 1251 | AGTTGGATGGCGCTCAAGAG |
| 1252 | TCCAACCAGATATGCTGAGT |
| 1253 | CGCTCAAGAGAGGTCAAGGC |
| 1254 | ACCTCCACTCTTTCTATAGG |
| 1255 | CCTGCTGAACTGCTTAACAT |
| 1256 | AAACTTTCCAGTGACCACAG |
| 1257 | TTGAATTTGTCCCTTTGAAA |
| 1258 | CTGCATCTCACTTGACCTCG |
| 1259 | CTGAACTGCTTAACATTGGA |
| 1260 | GCTGAACTGCTTAACATTGG |
| 1261 | CTCCTCCTTTTCACAGCAAT |
| 1262 | GCTTAACATTGGAGGGCCCC |

TABLE 54
Target sequences for GCH1 gene

| SEQ ID NOS | Target sequence |
|---|---|
| 1263 | CGCGATAGATCCTGTGGTATTGG |
| 1264 | GGGGTTACTTCGTACTATAATGG |
| 1265 | TAGTCTAAAGTCAACTTGATTGG |
| 1266 | CTACTAAGCATTAAGACAACAGG |
| 1267 | ATGGCGATTGAGCTGGGCGCAGG |
| 1268 | CACTACACCACTTTTATTGGAGG |
| 1269 | ATTGATGAGGTCGAGGAGCCGGG |
| 1270 | GTTTGGCTAAATGTTCGCACTGG |
| 1271 | GAACTTGGCCAATCAATCTTCGG |
| 1272 | GTTCAGGTGCGTGGAAGCTATGG |
| 1273 | ACTAACTGGAAGTTTTGCCCTGG |
| 1274 | TGGCGATTGAGCTGGGCGCAGGG |
| 1275 | CACCATTATGACGTTACTAAAGG |
| 1276 | TCTGTGCTCGTTCAGGTGCGTGG |
| 1277 | AGTGCATTTTCACAGATCGTTGG |
| 1278 | TGTAAGGCGCTCCTGAACTGTGG |
| 1279 | ATACGCTTTGGTTAAAACGTTGG |
| 1280 | GGTCCCTGATAGAACCAGAATGG |
| 1281 | AGGCAACGCGATAGATCCTGTGG |
| 1282 | GTTACCAAGCACCTCCATGGAGG |

TABLE 55
Target sequences for GJB2 gene

| SEQ ID NOS | Target sequence |
|---|---|
| 1283 | GCACTGATGGAACCGTCCTGAGG |
| 1284 | CCAAGTACAGGAGAACCGTGAGG |
| 1285 | GGCTACGTGATATTGCATGTAGG |
| 1286 | ATTTAGAGCATTLTTLCCGGCGG |
| 1287 | ACGCTGCAGACGATCCTGGGGGG |
| 1288 | TTGTCAAAGACCAACCCGTGGGG |
| 1289 | GACATAGAAGACGTACATGAAGG |
| 1290 | GTTCGCGAAGAGGTGGTGTGCGG |
| 1291 | GTCTTCTATGTCATGTACGACGG |
| 1292 | GCTCACAGGAGATTATCCACTGG |

TABLE 56
Target sequences for GJB6 gene

| SEQ ID NOS | Target sequence |
|---|---|
| 1293 | ACCCACTCATCATACCACGAGGG |
| 1294 | ACACGCAGCAAATGAAACGGGGG |
| 1295 | ACCGAGTCTTGGAATCACAATGG |
| 1296 | GTACCAATCTATAAAAACCAAGG |
| 1297 | TATCTCTTGACACTTGCGAGGGG |
| 1298 | TATGGCATAAAGTCTACTTGAGG |
| 1299 | AAACCAGCGCAATGGATTGGGGG |
| 1300 | ACGCTGCACACTTTCATCGGGGG |
| 1301 | ACCCTCGTGGTATGATGAGTGGG |
| 1302 | TCGCAGAAGGATAGACCCAATGG |

TABLE 57
Target sequences for GLA gene

| SEQ ID NOS | Target sequence |
|---|---|
| 1303 | ACCGAGATCTCACATGACGTAGG |
| 1304 | ACGGCCATAAAACTACACTGAGG |
| 1305 | ACGAAACGTTGAAAGCTGCGGGG |
| 1306 | ATAGCCATGAGCTTTCGAGGGGG |
| 1307 | GCCACACATACTGTACCACAGGG |
| 1308 | AGTGGGTTCGAACTTCAGCTCGG |
| 1309 | TCAATAAGGAGGGTATAAGGGGG |
| 1310 | CGATGGCAGAGTTACCGGTGAGG |
| 1311 | ACTGCGATGGTATAAGAGCGAGG |
| 1312 | TTAAGGAATAGAGCGGTGCAGGG |

TABLE 58
Target sequences for HBA Gene Cluster

| SEQ ID NOS | Target sequence |
|---|---|
| 1313 | AGAGTTTCACTGCATTAGCG |
| 1314 | TCCCGAGTAGCTGAGTAGCT |
| 1315 | ACATCTACAACTACTGCCAC |
| 1316 | CTGCCATAGGTGTTTACCAA |
| 1317 | GGGAAGGACATCACAAACGC |
| 1318 | ACAGTTGATACTGTACCCAC |
| 1319 | GGAGAAGGGACCTTCTAGCC |
| 1320 | GCCTGATCTTGACAGCCCCA |
| 1321 | CCAGCCTCAGGGGAGCTGAG |
| 1322 | CTCTCCAGTCGCAATGGGAC |
| 1323 | GTTTACCAAGGGTGATTCAT |
| 1324 | TGTTTACCAAGGGTGATTCA |
| 1325 | CTGCCATAGGTGTTTACCAA |
| 1326 | CTCTCCTCTCCAGTCGCAAT |
| 1327 | TTCCTATCAGTTGAGGGCCA |
| 1328 | AACCCTCCCTCTGATACCCC |
| 1329 | TGAGCATTCTGGGGTGACCT |
| 1330 | GTCTGGTGTGTGAGCATTCT |
| 1331 | AAGATATTCCTATCAGTTGA |
| 1332 | TCTGGTGTGTGAGCATTCTG |
| 1333 | GTGAGCATTCTGGGGTGACC |
| 1334 | TGTCTGGTGTGTGAGCATTC |
| 1335 | ATTCCTATCAGTTGAGGGCC |

TABLE 59
Target sequences for HBB gene

| SEQ ID NOS | Target sequence |
|---|---|
| 1336 | TTGGTTCTTCTATGGCTATCTGG |
| 1337 | CGGTTTGTTTCTATGGGTTCTGG |
| 1338 | GTAGACCTTATGATCTTGATAGG |
| 1339 | TACCTGTCTCAACCCTCATCAGG |
| 1340 | TTGTCTCTCCACATGGGTATGGG |
| 1341 | TAGACCTTATGATCTTGATAGGG |
| 1342 | AACCATCTCGCCGTAAAACATGG |
| 1343 | ATATCCCCCAGTTTAGTAGTTGG |
| 1344 | TCACACTAAGTAACTACCATTGG |
| 1345 | CCTAATTGTGTAATCGATTGTGG |
| 1346 | GATTACTGGTGGTCTACCCTTGG |
| 1347 | TTACCTCTATAATCATACATAGG |
| 1348 | CTTTCCTTACTAAACCGACATGG |
| 1349 | GGAGTAGATTGGCCAACCCTAGG |
| 1350 | GGCCAAGAGATATATCTTAGAGG |
| 1351 | GCGAGCTTAGTGATACTTGTGGG |
| 1352 | TGGTTATCAGGAAACAGTCCAGG |
| 1353 | CGTAAATACACTTGCAAAGGAGG |
| 1354 | GGGTTGGCCAATCTACTCCCAGG |
| 1355 | GAGTAGATTGGCCAACCCTAGGG |
| 1356 | ATCTCGCCGTAAAACATGGAAGG |
| 1357 | GCTGGCCCGCAACTTTGGCAAGG |
| 1358 | TGTATGATTATAGAGGTAAGAGG |
| 1359 | ACCGACATGGGTTTCCAGGTAGG |
| 1360 | AGCGAGCTTAGTGATACTTGTGG |
| 1361 | CTATCTTACTTACACATGAGTGG |
| 1362 | ACTATCAATGGGGTAATCAGTGG |
| 1363 | ACCACCAGTAATCTGAGGGTAGG |
| 1364 | GCATTTATGAGGTCAGCGTAGGG |
| 1365 | GACGAATGATTGCATCAGTGTGG |
| 1366 | AAGTCCAACTACTAAACTGGGGG |
| 1367 | TAAGTCCAACTACTAAACTGGGG |

TABLE 60
Target sequences for HEXA gene

| SEQ ID NOS | Target sequence |
|---|---|
| 1368 | TGGTTGACCCCACCTACAGGAGG |
| 1369 | ATTTACCACAGGCCCGCGTGCGG |
| 1370 | AGGAGGTCATTGAATACGCACGG |
| 1371 | TCCTTCTACATCCAGACGTGAGG |
| 1372 | TAGAAGGAAATGTCTCGTCGTGG |
| 1373 | AACCTGACCAATCTCCTTAGGGG |
| 1374 | GTCTGTATTTGGTGTCCGAGAGG |
| 1375 | CATGAGCTTTAAGTACGTAATGG |
| 1376 | GTAACATGAAAGTTATGACCAGG |
| 1377 | ATTACCCAGAAGCTTGTAGGAGG |

TABLE 61
Target sequences for HLA-A gene

| SEQ ID NOS | Target sequence |
|---|---|
| 1378 | TCCCTTGTCCGTTGTGTGAGCGG |
| 1379 | CTCACCTTTACAAGCTGTGAGGG |

TABLE 62
Target sequences for HLA-C gene

| SEQ ID NOS | Target sequence |
|---|---|
| 1380 | AGGCTGAAAACTACACATCCCGG |
| 1381 | GTAAGCGATGACACTCTGAACGG |
| 1382 | CGAGGCTGATGCAGACATGTGGG |
| 1383 | CTATATGTGGAGGTGGCATCTGG |
| 1384 | CATGTGGGATCCTGGTGTTCTGG |
| 1385 | TTGGAGTGGCATTGTGTGCTTGG |
| 1386 | GATGCAGACATGTGGGATCCTGG |
| 1387 | TAAGAGGTCACACCACATAAAGG |
| 1388 | CACGGATGTACTCACCAGTTGGG |
| 1389 | ACCGCACAGCAGGTCACTAGTGG |
| 1390 | GCACGTCTGTTTATAGGCTCTGG |

TABLE 63
Target sequences for HTT gene

| SEQ ID NOS | Target sequence |
|---|---|
| 1391 | ATATACAGTACGTTAATACGTGG |
| 1392 | TAATTGCCGAGGGATGAATGAGG |
| 1393 | TTATTCCAACCCATCCAGGGAGG |
| 1394 | TTTTGCAGTGATACGTCTGGGGG |
| 1395 | TGTAATCGTTGATATACGTGAGG |
| 1396 | AGTAAAGTGGTGAACTTACGTGG |
| 1397 | CCTGTCCTGAATTCACCGAGGGG |
| 1398 | CTTAGAAATCTTTCACCGAGGGG |
| 1399 | AGTAGTGGTATTCCAGATGGGGG |
| 1400 | TGTATCGTCACACGTTCTGTGGG |

TABLE 64
Target sequences for IKBKG gene

| SEQ ID NOS | Target sequence |
|---|---|
| 1401 | AAGACGAGGAGGGTTAAACGAGG |
| 1402 | CGAGTCACTTACAAACAAAGTGG |
| 1403 | CGGGGGCTCATGAGTCACCGGGG |
| 1404 | GCCGTGAGTTGATGTGACATGGG |
| 1405 | GGGGATTCGGGAGCACTACGCGG |
| 1406 | AAGTGTACGACCGTTTCCGGGGG |
| 1407 | CCTAAGTGTCCACCCCATCGTGG |
| 1408 | AACCGAGTAAAATCCTTGTGGGG |
| 1409 | GCTTTTGAAACGAGGGCCCAGGG |
| 1410 | ATAATGGGAGAGGATTGCGAGGG |

TABLE 65
Target sequences for IKZF1 gene

| SEQ ID NOS | Target sequence |
|---|---|
| 1411 | GGGTGTCGTAAACAAAACAGAGG |
| 1412 | GGTTTAGAGAGACGTACCAGCGG |
| 1413 | TGACTTGAGCGTCAAACCTGCGG |
| 1414 | CTACGCAAAACTCAGCACAAAGG |
| 1415 | GGTTAACGAAGAATTCATCAAGG |
| 1416 | ATTGAACCCCGATATCAGTGAGG |
| 1417 | TGGCACTCACCAACCAACCGAGG |
| 1418 | GCGTCACCCCAAAGTTTGCGGGG |
| 1419 | GTAGTGCTAAAGGATTTCTGTGG |
| 1420 | CGGAAGCATAAACACTCTGGTGG |

TABLE 66
Target sequences for JAK2 gene

| SEQ ID NOS | Target sequence |
|---|---|
| 1421 | GTGCACTTACTCACATCACATGG |
| 1422 | GCGCCATCTCACACTTACTGAGG |
| 1423 | TTGCTTATACTTTCCCTACGTGG |
| 1424 | TGTGTAACGTATGTACAGACTGG |
| 1425 | AGACAGTTGAGCGTATATTGTGG |
| 1426 | TCGATAACTTATAAATCTGAGGG |
| 1427 | GCCCGGTCTCCTGCCATTCGGGG |
| 1428 | GGCAGCACAATAATTGGTAGGGG |
| 1429 | GGTTTGCTTTTCAGTGACGGAGG |
| 1430 | GGGGCAGCACAATAATTGGTAGG |

TABLE 67
Target sequences for KCNH2 gene

| SEQ ID NOS | Target sequence |
|---|---|
| 1431 | GGGGTATAAAGTCTCCACGGGGG |
| 1432 | CCCTCCACTGAAAAACGACGGGG |
| 1433 | GGCTCCATCGAGATCCTGCGGGG |
| 1434 | TCGCCCGGGATACCTGACAGGGG |
| 1435 | CCGATGCGTGAGTCCATGTGTGG |
| 1436 | AGTCAACAAACCCACCTCCGAGG |
| 1437 | GTCAACAAACCCACCTCCGAGGG |
| 1438 | ACTGGCACATTTGCTGACGTGGG |
| 1439 | CTCTAACTCCGTACTGCCGGGGG |
| 1440 | ACCATCGTGACATGGTTTGGGGG |

TABLE 68
Target sequences for KCNQ1 gene

| SEQ ID NOS | Target sequence |
|---|---|
| 1441 | GTGCTGTAGATGGAGACGCGCGG |
| 1442 | AGTTATCTTACTGCACCCAAGGG |
| 1443 | CGGGATAGATGACACGAGCGGGG |
| 1444 | GCTCGAGGAAGTTGTAGACGCGG |
| 1445 | TTTGGCTCCACACCTCCGGGAGG |
| 1446 | GGGTGCGTGTTAATCAACAATGG |
| 1447 | ACGAGCGGGGCTAAGCAGGTGGG |
| 1448 | CAGGCGGGGTAAATGCACACTGG |
| 1449 | CGGTCTTTATGAGCATGCGGGGG |
| 1450 | GAACCTTTGCATATAACGTGCGG |

TABLE 69
Target sequences for KLF5 gene

| SEQ ID NOS | Target sequence |
|---|---|
| 1451 | GAAGTTGTGTACAAACTGCGCGG |
| 1452 | ACCCGTACCTACATAAGACGGGG |
| 1453 | CCCCAAGGTTTCATACCCGGTGG |
| 1454 | TTTACTCTCAGCGAAACGCGGGG |
| 1455 | GTTTCGCTGAGAGTAAATGGGGG |
| 1456 | TGCGTCGTTTCTCCAAATCGGGG |
| 1457 | GTCAAGTGTCAGTAGTCGCGGGG |
| 1458 | TTAAGGTCTCGTGCATTACGTGG |
| 1459 | TGGTACTGATAACTTCACATTGG |
| 1460 | AATGGTACAGCACTACTAAGCGG |

TABLE 70
Target sequences for KRAS gene

| SEQ ID NOS | Target sequence |
|---|---|
| 1461 | GATTAGGTCAAATCCCTTTATGG |
| 1462 | AATACGCATCGTGTTATCTCTGG |
| 1463 | GCTTACTATTCAACTCTAACAGG |
| 1464 | AACTTTTTCGTTCCACGTACTGG |
| 1465 | CCTACTGTCGCTAATGGATTGGG |
| 1466 | TCCTACTGTCGCTAATGGATTGG |
| 1467 | TAGTTACTACTCAGTTGAACAGG |
| 1468 | TATACTTACGTAAAATCCATTGG |
| 1469 | GCAATGTCATGAGTGAATACTGG |
| 1470 | CACCTATCCTACCCACGAATTGG |
| 1471 | ATACGCATCGTGTTATCTCTGGG |
| 1472 | ACTTTTTCGTTCCACGTACTGGG |
| 1473 | ACGCACCCTGAAATTGGAAGTGG |
| 1474 | TGCCAATTCGTGGGTAGGATAGG |
| 1475 | CGCCGAATGGTGACAGCAAGAGG |
| 1476 | GGGCAATGTTCATGAGTGCTGGG |
| 1477 | AAGGCTGCCAATTCGTGGGTAGG |
| 1478 | ATGACTTAGGTTTGCCAATGTGG |
| 1479 | ATCTCTGGGTCGTATACCAAAGG |
| 1480 | AGTATTCCATATCCATTTCGGGG |
| 1481 | GTTTAAAGTGACCCCAACACAGG |
| 1482 | AGTAGAGTGTGTGCGCCGAATGG |
| 1483 | GCTTTTTAGATCTGTATACGTGG |
| 1484 | TATAACTATATCCCAGTACGTGG |

TABLE 71
Target sequences for LCA5 gene

| SEQ ID NOS | Target sequence |
|---|---|
| 1485 | GGGTCACTGGGAAACTTATAAGG |
| 1486 | gaataacttcagaccgagtttgg |
| 1487 | CAATGAGCAGGTGCAGTATATGG |
| 1488 | TACGGTAGTTTGATGTGATATGG |
| 1489 | TCCCCTAATGAGTTCGCATTTGG |
| 1490 | TATCGTCTGCATGTTTTAATCGG |
| 1491 | AGAACTCCATGTCGTAAAACAGG |
| 1492 | GCGAACTCATTAGGGGAGGCTGG |
| 1493 | ACCCCTGGCCCTATCCATAAAGG |
| 1494 | ACGGGTTCAGTGACATAAGAAGG |
| 1495 | CGAGTAGTACTTTAGAATAGTGG |
| 1496 | CTCTATGGAATACCTCCGTATGG |
| 1497 | CCGATACGTTGTTTTCTTTGGGG |
| 1498 | TTGATGACCTTGGATCATGCTGG |
| 1499 | GAAAACGTTAGTTACTGTACAGG |
| 1500 | TATTCTAAAGTACTACTCGTTGG |
| 1501 | CGGGGATTCCTTAACTACCATGG |
| 1502 | TATGGCAAGTTTAACTGCACTGG |
| 1503 | GTAGCCCTCTTGTGTATGGTTGG |
| 1504 | TTCTAAAGTACTACTCGTTGGGG |
| 1505 | GCGTGGAGAGTAAACCAGACAGG |
| 1506 | AGGTCTGTTCTATACGAAGTGGG |
| 1507 | ATGCAACCCAAAAGTTCCGTGGG |
| 1508 | TATGCAACCCAAAAGTTCCGTGG |
| 1509 | TGCTTTACACATTATGACCGGGG |
| 1510 | CAGGTCTGTTCTATACGAAGTGG |
| 1511 | TGATGACCTTGGATCATGCTGGG |
| 1512 | AAAACGTTAGTTACTGTACAGGG |
| 1513 | AAATGCGAACTCATTAGGGGAGG |
| 1514 | GGTTACCCATGAGATTACACAGG |
| 1515 | ACTCCATGTCGTAAAACAGGAGG |

TABLE 72
Target sequences for LRRK2 gene

| SEQ ID NOS | Target sequence |
|---|---|
| 1516 | CAACGTCTGTTCAGCTTACGTGG |
| 1517 | CGTCTGTTCAGCTTACGTGGAGG |
| 1518 | CTGTTCAGCTTACGTGGAGGAGG |
| 1519 | TCATCCGTTCTTATACAATCTGG |
| 1520 | CTATAGAACTATACTTGACATGG |
| 1521 | TTCATCTCCGGTTTGAAATCAGG |
| 1522 | ACATGCCAATTGTCTAAATAAGG |
| 1523 | GGTACAATGCAAAGCTTAATGGG |
| 1524 | GCTTGATACACCCAGATATAAGG |
| 1525 | GGCTCCCCGCTTTCATCCTAGGG |
| 1526 | GCTCCCCGCTTTCATCCTAGGGG |
| 1527 | ACCCAATATCCAGGTTGAGTAGG |
| 1528 | CCAGTTCCCAGACCTTCCGTGGG |
| 1529 | TTCTGCGCGGCCCGTCGCCTCGG |
| 1530 | GGCCCCTGAGCTCGTTTTTGGGG |
| 1531 | GGCCCCAAAAACGAGCTCAGGGG |
| 1532 | TCCTCATAAACAGGCGGGCGTGG |
| 1533 | GTGTTCACGTACTCCGAGCGCGG |
| 1534 | TTTCAAGTGATTACCGCGCTCGG |
| 1535 | GAGTCCAAGACGATCAACAGAGG |
| 1536 | GAGAGTCGCGAGTGTGCAGCAGG |
| 1537 | CGCGAGTGTGCAGCAGGTAAAGG |
| 1538 | ACAAGGTATACTACAACTAAAGG |
| 1539 | GGGTAGGCGTTTTGGTCTGCAGG |
| 1540 | AGTGCTATACTTGACAACCCAGG |
| 1541 | GTGCTATACTTGACAACCCAGGG |

TABLE 73
Target sequences for MDM4 gene

| SEQ ID NOS | Target sequence |
|---|---|
| 1542 | GGGATATTATCGTTAAATATAGG |
| 1543 | ACTCCAACAACTTACTCATTGGG |
| 1544 | ATAGGGCCAGTTAGGGAGCGTGG |
| 1545 | AGTTAGGGAGCGTGGTTCATTGG |
| 1546 | AGATAGGGAATACAAGCGGTTGG |
| 1547 | GATAGGGAATACAAGCGGTTGGG |
| 1548 | TATAGGAACCTTAAGTCAGCGGG |
| 1549 | ATAGGAACCTTAAGTCAGCGGGG |
| 1550 | GGTGCATCCGTTACTATTATGGG |
| 1551 | CTCGTGTGAGGCCGTGTGGGAGG |
| 1552 | CGGCCGTACCGCCAGTTGTGCGG |
| 1553 | GGCCGTACCGCCAGTTGTGCGGG |
| 1554 | GTGAAGTAACTTTGGCCAACAGG |
| 1555 | AAACCTAAAGTCGACGTAGTTGG |
| 1556 | CACGTCAATGTCATTCTACCCGG |
| 1557 | ACCTACAGACAGTATCGAGATGG |
| 1558 | CCTACAGACAGTATCGAGATGGG |
| 1559 | CTACAGACAGTATCGAGATGGGG |
| 1560 | CGGGTGTTGCTTTTAAACTGTGG |
| 1561 | GTAACTTGCAGTTAGTAGGTAGG |

TABLE 74
Target sequences for MET gene

| SEQ ID NOS | Target sequence |
|---|---|
| 1562 | ATAACTGTTTGATAAGACCGTGG |
| 1563 | CCATAACATTCTCCTAACAGTGG |
| 1564 | CAATTTTTTGACAACCTACGAGG |
| 1565 | AACTCTTCATCAGCTAACCAAGG |
| 1566 | AGGTCGTTTTGGTATCAGAAAGG |
| 1567 | CAAATCTCTCTAAACCCGGGTGG |
| 1568 | CAAAGCTCGCGCCCTTCCCGGGG |
| 1569 | TGTCAGTTCCTATTGGCACGTGG |
| 1570 | CTATTATGTAGATCTGCAGAAGG |
| 1571 | GGTAGAGTATCATATGTGCTAGG |

TABLE 75
Target sequences for MLH1 gene

| SEQ ID NOS | Target sequence |
|---|---|
| 1572 | TCTTGTACTACAAAGCCTTA |
| 1573 | CAGTTTGGACGGCTGGTACT |
| 1574 | TTGTGATCAGTTTGGACGGC |
| 1575 | AGTTGTGGCAACCCGAAACA |
| 1576 | CAGTTGTGGCAACCCGAAAC |
| 1577 | ACCGGGCTCCATTTCAGTTG |
| 1578 | CTCACAAGGTCATCCCAACC |
| 1579 | TCTCACAAGGTCATCCCAAC |
| 1580 | TGGCAACCCGAAACAGGGCT |
| 1581 | TTAATTGTGATCAGTTTGGA |
| 1582 | TACCTATAAGAATACTCATC |
| 1583 | ACCTTAAACAAGGCCAGACG |
| 1584 | TGGGTAGAAAGATATCCAAC |
| 1585 | CTAGATAGGACTATATTTAC |
| 1586 | GTAGCCATTAAAACCTAGAT |
| 1587 | ACTCATCAGGACCTTAAACA |
| 1588 | TAGATAGGACTATATTTACT |
| 1589 | TCAGGAGTTCAAGACCAGCC |
| 1590 | GTCCTGATGAGTATTCTTAT |
| 1591 | CAGTAAATATAGTCCTATCT |
| 1592 | CTTGGCCTTGCAAAGTGCTG |
| 1593 | ACCACGTCTGGCCTTGTTTA |
| 1594 | ATGGTGATTTTTACATGCAG |
| 1595 | TGCAGAGGGGAGCAACTATG |
| 1596 | TGGTGATTTTTACATGCAGA |
| 1597 | CAACACGAATCTAGTCTTTA |
| 1598 | ATCCATATACCTCCCATATA |
| 1599 | TATAAAGTCCTGAGACCGCT |
| 1600 | AGACCGCTAGGAATCTATGA |
| 1601 | TGATTCACGCCACAGAATCT |
| 1602 | CACAAAGCCTGGAATATGAG |
| 1603 | AACACGAATCTAGTCTTTAA |
| 1604 | CGAATCTAGTCTTTAAGGGC |
| 1605 | CTAACTTCTAGCACAAAGCC |
| 1606 | GAGACCCTTCCATATATGGG |
| 1607 | TACTGAGACCCTTCCATATA |
| 1608 | GTGAATCATGTGTTCTTTCA |
| 1609 | GGGGAAAAGTGCTTGCATTA |
| 1610 | TTTCCATCATAGATTCCTAG |
| 1611 | ACTGAGACCCTTCCATATAT |
| 1612 | TTAATTGCCTCTCATATTCC |
| 1613 | CAGGCTTTGTGCTAGAAGTT |
| 1614 | AGGCTTTGTGCTAGAAGTTA |
| 1615 | CCAACCCCTTGGACCTCAAC |
| 1616 | ATGATCTCTGGCCAACCCCT |
| 1617 | CAACCCCTTGGACCTCAACT |
| 1618 | CCATTCTGATATTGCAACCA |
| 1619 | TACTCAACTATTAGTGAATG |
| 1620 | CATACTCAACTATTAGTGAA |
| 1621 | GAGCAAATGGCAATCACTCT |
| 1622 | TCCATTCTGATATTGCAACC |
| 1623 | ATACTCAACTATTAGTGAAT |
| 1624 | CACTTCTCCCAAATCACTGT |
| 1625 | AGAGCAAATGGCAATCACTC |
| 1626 | CTGAGAGGTTCTTCTCCCCA |
| 1627 | TCCATCTTCATTTCACACTT |
| 1628 | TATGTAGATTTGCTAGGACC |
| 1629 | GTTAGGGCAAGTGGCGGTGA |
| 1630 | TCTGTCCCAGTTGAGGTCCA |
| 1631 | CCAGTTGAGGTCCAAGGGGT |
| 1632 | CTGTCCCAGTTGAGGTCCAA |
| 1633 | CAAATAGAGAGGTTTTCATC |
| 1634 | CTACAGTGATTTGGGAGAAG |
| 1635 | TGTCCCAGTTGAGGTCCAAG |
| 1636 | AAGTGGCGGTGATGGAGTTG |
| 1637 | AAGGGGTTGGCCAGAGATCA |
| 1638 | GTTGAGTATGTAGATTTGCT |
| 1639 | CATTTGCTCTGTCCCAGTTG |
| 1640 | GGATCTGTTAGGGCAAGTGG |
| 1641 | GTCTGAGTATGGATCTGTTA |
| 1642 | CCCTGGTTGCAATATCAGAA |
| 1643 | GTTGCAATATCAGAATGGAT |
| 1644 | TATGGATCTGTTAGGGCAAG |
| 1645 | GGTCTGAGTATGGATCTGTT |
| 1646 | TGGTGAGGGACTTGAGACAC |
| 1647 | GATGGAAGCCTACAGTGATT |
| 1648 | CATGGTGTTCTTTAAGGCAG |
| 1649 | TGAAATTAAGTGTGGATATC |
| 1650 | CTGGAGGCAAAAAACGTTAA |
| 1651 | TCACTTCCTACTTCTGAGCT |
| 1652 | ACCCTGGCTTTCTGCTGAAC |
| 1653 | CCATGGTGTTCTTTAAGGCA |
| 1654 | TCTGGAGGCAAAAAACGTTA |
| 1655 | ACCATGGTGTTCTTTAAGGC |
| 1656 | TGTCACCTAGTGACAAACCA |
| 1657 | GGCCAGTTCAGCAGAAAGCC |
| 1658 | TGCTCTTTGGTGAACAGTCC |
| 1659 | GCAAAGGCACTGGCATACAG |
| 1660 | TATATCCATGGTTTGTCACT |
| 1661 | GCTCTTTGGTGAACAGTCCT |
| 1662 | GGTCTGAATGTATATATCCA |
| 1663 | AAGGGTGTCTTGATCATCTC |
| 1664 | ATGGTTTGTCACTAGGTGAC |
| 1665 | AATAATCAAAAGTAGACCTA |
| 1666 | GAGAAGTAATCCCTGAAACA |
| 1667 | ATGTTCTGTCCCTACCTGTC |
| 1668 | TTCCCAACGTCTTCAACCAG |
| 1669 | GATTCCCAACGTCTTCAACC |
| 1670 | ATTCCCAACGTCTTCAACCA |
| 1671 | TGCTTGAGATACAACCAGTT |
| 1672 | CTGGCCAGCCTCTAACAGAC |
| 1673 | TCTTCAACCAGGGGTCTGAC |
| 1674 | TAGAGCACTAAGACCAAGTC |
| 1675 | TGTGAATGGTTTTCCAGTAA |
| 1676 | GAATAAGTCAGCTACTCAAT |
| 1677 | GAAGTCTTTAAGCAAGTCTA |
| 1678 | TGAATGGTTTTCCAGTAAGG |
| 1679 | GTTTAAGGGAATGACCTCCA |
| 1680 | CGGGTTCAGAGTTCAATATC |
| 1681 | GTGAATGGTTTTCCAGTAAG |
| 1682 | AGTCTTTAAGCAAGTCTATG |
| 1683 | CACCAAAATGCAGACATAGA |
| 1684 | ATGTGAATGGTTTTCCAGTA |
| 1685 | CAGCTGTTAATAAATGTGAA |
| 1686 | GGGGAAAATCTAGTGACTAA |
| 1687 | TCACCCAGGCTGGAGTGCAG |
| 1688 | TCTTCTGCCTCAGCCTCCCG |
| 1689 | TTTCGCTCAGTCACCCAGGC |
| 1690 | GGAGTGCAGTGGCACGATCT |
| 1691 | AATAAAACTAGACTTTAAAA |

TABLE 76
Target sequences for MSH2 gene

| SEQ ID NOS | Target sequence |
|---|---|
| 1692 | AAGACCCATTATGTGTGGGC |
| 1693 | GGTATTTCAACGTTTGGCCT |
| 1694 | GTCTGTGGTATTTCAACGTT |
| 1695 | ACACTCAAGCTATAGGTCAT |
| 1696 | GGTAAACTAACAATCGAAGG |
| 1697 | TAATTTAACGACCCACTACT |
| 1698 | TGAGTCATCTGTAATGCCTA |
| 1699 | GCAAGGTGTGACCCAGTAGT |
| 1700 | TGCAAGGTGTGACCCAGTAG |
| 1701 | TTAGGGAGTTCCTAATGACC |
| 1702 | TAGGGAGTTCCTAATGACCA |
| 1703 | GGTCATTAGGAACTCCCTAA |
| 1704 | GTTGAATTTTAGGTGTACCC |
| 1705 | TTAGGTGTACCCTGGTCATT |
| 1706 | GCCATGGCAATTTGTTCCCG |
| 1707 | TCCACGGGAACAAATTGCCA |
| 1708 | TACCTACAGTATACTTACCT |
| 1709 | TGCCTAGGTAAGTATACTGT |
| 1710 | CCAAGACATTAGTACGTTGT |
| 1711 | GTTACAGTAGGACACATAAC |
| 1712 | CTACAACGTACTAATGTCTT |
| 1713 | CCTACAACGTACTAATGTCT |
| 1714 | GATTCCACTTGGATATACGT |
| 1715 | CACTTGGATATACGTTGGAG |
| 1716 | CGTTGGAGTGGAATTGTCTG |

TABLE 77
Target sequences for MSH6 gene

| SEQ ID NOS | Target sequence |
|---|---|
| 1717 | TAGTTCAACCTAGTATAAGG |
| 1718 | ACATAGTTCAACCTAGTATA |
| 1719 | GGGTGGTTGTAAACCAGACA |
| 1720 | GTAAACCAGACAAGGCCACC |
| 1721 | GTTTACAACCACCCCTTTGA |
| 1722 | TTGTATAGGTGCTACTAATT |
| 1723 | TCGAGCCTTTTCATGGTCAA |
| 1724 | GGACTTATTACTCCCAAAGC |
| 1725 | CGTGTTTAAGACTGTAACTG |
| 1726 | ATCCCATGCATGATTTCTAC |

TABLE 78
Target sequences for MUTYH gene

| SEQ ID NOS | Target sequence |
|---|---|
| 1727 | CAACTCCGGACGATCAGCCC |
| 1728 | TGAGCCGGACTCCCCAACTC |
| 1729 | TAAACCGAACTTTGGCCAGA |
| 1730 | TCACAGGTATTGTGTACCTC |
| 1731 | GCTGAACTCAAGAAGCCGCA |
| 1732 | ATTTCCTCACCATTTCCGGA |

TABLE 79
Target sequences for MYC gene

| SEQ ID NOS | Target sequence |
|---|---|
| 1733 | CTTCGGGGAGACAACGACGGCGG |
| 1734 | GCCGTATTTCTACTGCGACGAGG |
| 1735 | ACCCCTCCATAAATACAAGGGGG |
| 1736 | TCCGTATTGAGTGCGAAGGGAGG |
| 1737 | TAAGTGATCAGACACCGTCAGGG |
| 1738 | GCGCGCGTAGTTAATTCATGCGG |
| 1739 | GGCGGGTTGGAATCGCCGCGGGG |
| 1740 | TGCGTAGTTGTGCTGATGTGTGG |
| 1741 | GTCAAACAGTACTGCTACGGAGG |
| 1742 | CGAGGGGTCGATGCACTCTGAGG |

TABLE 80
Target sequences for MYCN gene

| SEQ ID NOS | Target sequence |
|---|---|
| 1743 | AAGCGAGTTAAACAACCCTGTGG |
| 1744 | ACAACACGCAGTCAAAGCGGGGG |
| 1745 | ACATACGAGCACTAACAAAGGGG |
| 1746 | GCTCCCCAACTGGTACAACGAGG |
| 1747 | TCGCACACCCTTGAGATACGAGG |
| 1748 | CTCCCCAACTGGTACAACGAGGG |
| 1749 | AGAAATCGACGTGGTCACTGTGG |
| 1750 | CTTTCTGCTCAGTCTCCGCGAGG |
| 1751 | TCCATGACAGCGCTAAACGTTGG |
| 1752 | TCACCAACCTCGTATCTCAAGGG |

TABLE 81
Target sequences for MYH11 gene

| SEQ ID NOS | Target sequence |
|---|---|
| 1753 | TTGTGTTGCACTAACCCAAGCGG |
| 1754 | TTATACGTGTTAATCCAAGGTGG |
| 1755 | GATGCTCAAATTCAGCGCAGAGG |
| 1756 | GATTTCCTACTTCCTACAAGCGG |
| 1757 | TGTTGCACTAACCCAAGCGGAGG |
| 1758 | TACACTCAAGATGATTCCCGAGG |
| 1759 | GTGGGATTTCCAACGCACCATGG |
| 1760 | AACTTTGAGACCTTTACACGTGG |
| 1761 | TGTGACGAAGAGAGCTGTGTGGG |
| 1762 | CCTTGTCAAAGACGTGAACGTGG |

TABLE 82
Target sequences for NPC1 gene

| SEQ ID NOS | Target sequence |
|---|---|
| 1763 | GTGGTAGGTCATGAAGTACGTGG |
| 1764 | CGTCCGTTCTGTCCACGATGTGG |
| 1765 | TGCCGAGCAGAGTTATGCGATGG |
| 1766 | AGCGAAACCAGCGTTTGCGAGGG |
| 1767 | TTATGCTCTGGAACTCACCGAGG |
| 1768 | GAGAAATATTAATCCGTGAGTGG |
| 1769 | CCTGTAAGGAAATACTCGGTAGG |
| 1770 | GTACAGTAAGATTGGTGTGATGG |
| 1771 | CCAACCGCACATCACACGCTGGG |
| 1772 | GGGTTATCCGAAAGGAACATGGG |

TABLE 83
Target sequences for NPC2 gene

| SEQ ID NOS | Target sequence |
|---|---|
| 1773 | AGCTGCCAGGAAACGCATCGCGG |
| 1774 | AACCCCGACGACAGGCAAGGAGG |
| 1775 | CAGATGCACCGAACTCAATGAGG |
| 1776 | TACCACTTAACACTGAACAGAGG |
| 1777 | TGCGCGGTCGGGTTTCATGGAGG |
| 1778 | GGCTTTTGGAAATCACCGAAGGG |
| 1779 | ATCAACCCCGACGACAGGCAAGG |
| 1780 | GGGTTCCCTAAATCTTAAGGAGG |
| 1781 | TAGTCGGTAGAAAGTCAGGCCGG |
| 1782 | CGGTCACAAGACAAACCTGTCGG |

TABLE 84
Target sequences for OTOA gene

| SEQ ID NOS | Target sequence |
|---|---|
| 1783 | AAGTTGGCAATTCCAGTAGAGGG |
| 1784 | AACTGGGTATCCCTGATATGAGG |
| 1785 | GTACCCATTGGTGTTATCTTAGG |
| 1786 | GTACAAAGTCCTAACACCCCTGG |
| 1787 | TCAACTGAAGCTCCCACGTGTGG |
| 1788 | AGCACAAGCGTGTTGATAGGTGG |
| 1789 | AGCGTGTTGATAGGTGGCAAAGG |
| 1790 | CAGTCATGATACTACCCACAAGG |
| 1791 | AATGGGGGAATCGGGCTGGCTGG |
| 1792 | CCTAAAAGGGGATGTGCGCCCGG |
| 1793 | TTAAATGTTGGCGGCTAATGAGG |
| 1794 | TCTCCCAACACCCCAAATACAGG |
| 1795 | CTCATACGACACAGTGATGCTGG |
| 1796 | ATGTATCAGCTACCCTAATCAGG |
| 1797 | CGTGATTCAGAACAGGTGACTGG |
| 1798 | TACTATGATCGATAAGAAATAGG |
| 1799 | CTATGATCGATAAGAAATAGGGG |
| 1800 | TCGATAAGAAATAGGGGTCTTGG |
| 1801 | CGATAAGAAATAGGGGTCTTGGG |

TABLE 85
Target sequences for PAH gene

| SEQ ID NOS | Target sequence |
|---|---|
| 1802 | GCAGCTTATAGGTTCACCAGAGG |
| 1803 | CTGTGATGTAGAAGGAATCGGGG |
| 1804 | TCCGTTTTGATATGCAACCTGGG |
| 1805 | ATCCGTTTTGATATGCAACCTGG |
| 1806 | TAAGTAATTTACACCTTACGAGG |
| 1807 | GCTACGACCCATACACCCAAAGG |
| 1808 | TATTATGGCCCTTGTGACCATGG |
| 1809 | TGATTTACCCCTACCCTACTAGG |
| 1810 | TCATTTTAGGCCACACCAAGTGG |
| 1811 | AAGTATTACAGACGCACTGGTGG |
| 1812 | ACTTGGTGGTTGCGTTGAACAGG |
| 1813 | CTTGGTGGTTGCGTTGAACAGGG |
| 1814 | AACTCTCTGCCACGTAATAGAGG |
| 1815 | ACTCCGTGACAGTGTAATTTTGG |
| 1816 | CGTGACAGTGTAATTTTGGATGG |
| 1817 | AGCTCATTAGGCACAACAGTGGG |
| 1818 | TCAGTACTGGCAACAAATGTGGG |
| 1819 | GTTCTACTCCAATATATGGCAGG |
| 1820 | ATATGGCAGGGTGGGTCTTAGGG |
| 1821 | AGGGTGCATACACACTTTACTGG |
| 1822 | CCCAGCTGGCATATATAAGCAGG |
| 1823 | TAACACCCCATCAGTGGATCAGG |
| 1824 | TCGATTACTGAGAAACCGAGTGG |
| 1825 | ACCTCAATCCTTTGGGTGTATGG |

TABLE 86
Target sequences for PCCA gene

| SEQ ID NOS | Target sequence |
|---|---|
| 1826 | GTTACCTAATGAGACCATGGGGG |
| 1827 | CTTATCGACATGGAAGTGAGTGG |
| 1828 | TTTGTGTCCAATTCAGCGTGCGG |
| 1829 | AGTACCACATCGAACTGGAAAGG |
| 1830 | CGAGTCTGTCGTTAATTCTGGGG |
| 1831 | TGTTCCTCGCGGGGATCCTGCGG |
| 1832 | GAAGTTCACTATCACTCTAGGGG |
| 1833 | TCCCCTTTCCGCAAGTTAGGGGG |
| 1834 | CGTTGCAGCTGTTCCTCGCGGGG |
| 1835 | GCCAGTAGTTGTACTAACAAGGG |

TABLE 87
Target sequences for PCCB gene

| SEQ ID NOS | Target sequence |
|---|---|
| 1836 | CGTGCCCCATGAAAGAGTGATGG |
| 1837 | ACATGCGTACTCAGGTGCGCCGG |
| 1838 | TGTGCGCGTGCAGGAACTTGTGG |
| 1839 | GTACTCAGGTGCGCCGGTAGGGG |
| 1840 | GCGACCTATCACTGCGTGCCCGG |
| 1841 | AACGCATCGAAAACAAGCGCCGG |
| 1842 | CGCATTTGACAAGGGTCCAAAGG |
| 1843 | AAGGTCAAGAGTACCCATTACGG |
| 1844 | GCGTACTCAGGTGCGCCGGTAGG |
| 1845 | GTCACAGATACCAGGATACTGGG |
| 1846 | CGGCACAGCAAAAATGGCGGCGG |
| 1847 | GTACTTGCATTGAGATCAACGGG |
| 1848 | TCCGTAGATTTTCCCAGAAGAGG |
| 1849 | TTGCCCAGTGTGTCCGTGACTGG |
| 1850 | TGAATGACCCTGTGTTATCCAGG |
| 1851 | TGGTATTAAAGGGCAATTACTGG |
| 1852 | GGCTACTCTCGATGTTTGGCTGG |
| 1853 | CGTACTCAGGTGCGCCGGTAGGG |
| 1854 | TACTTCTAACCTACTCTGTTAGG |
| 1855 | TGGAGCATAGTGGTATTAAAGGG |
| 1856 | CGCAGGCTACTCTCGATGTTTGG |
| 1857 | CGGGGCAAGGCTCAGCGTTCTGG |

TABLE 88
Target sequences for PHEX gene

| SEQ ID NOS | Target sequence |
|---|---|
| 1858 | TGGGTGTAAGTGGCTTCGAGTGG |
| 1859 | ATCGGTTGAAAGATTCTCCGCGG |
| 1860 | TATCTTGCGTATGTTTCCGAGGG |
| 1861 | CCTGTCGGTAAGTGATGGGTAGG |
| 1862 | AGGGTCGTCGTCTCTTCAAGGGG |
| 1863 | TATATCGTTAGTGAAAGGCCTGG |
| 1864 | CTAAACCATCCATACAGATACGG |
| 1865 | AATTCCTGTCGGTAAGTGATGGG |
| 1866 | TTTATCTAACGATGAGCAGAAGG |
| 1867 | TTTCCGTGTTACTTTAAGTGTGG |

TABLE 89
Target sequences for PIK3CA gene

| SEQ ID NOS | Target sequence |
|---|---|
| 1868 | AGCAAGCACATCCACAGCGTAGG |
| 1869 | GTAAAGGGAGCGCAACAAGAGGG |
| 1870 | AACTGTACATAAACTTCGGGCGG |
| 1871 | CCCCGAGCGTGAGTAGAGCGCGG |
| 1872 | GTAAACACCAGACGTTCAGCCGG |
| 1873 | AAGGTATAGGTACTCAGGAGAGG |
| 1874 | GGGTGTCATGTATAATACAGAGG |
| 1875 | GTGTCATGCATTCAAGTACCAGG |
| 1876 | CGATCACGAATCAGAAAACACGG |
| 1877 | CGAGTATTATGAGATTACCTGGG |

TABLE 90
Target sequences for PKD1 gene

| SEQ ID NOS | Target sequence |
|---|---|
| 1878 | GCTGCCGTCAGAAATCCCCGCGG |
| 1879 | CGGCAGAAAGTAATACTGAGCGG |
| 1880 | GACCGGGCATATCAGCATGGTGG |
| 1881 | ACGCAACACTCACGCCCGGGGGG |
| 1882 | CGGCGGTGTTAAGAGGGCAAAGG |
| 1883 | CCGATATCTACCCCTCCAAGTGG |
| 1884 | CACGCAACACTCACGCCCGGGGG |
| 1885 | CCGAAGCACTGTCCGAGCAAGGG |
| 1886 | GGCAGCGAAGACACGTTGAGGGG |
| 1887 | GGGCGTACCGAGGTGAGCAGAGG |

TABLE 91:
Target sequences for PLP1 gene

| SEQ ID NOS | Target sequence |
|---|---|
| 1888 | ACTTAAATCTAAATGCACCGGGG |
| 1889 | GTGCACACTATGAGGAATCGGGG |
| 1890 | CAATGGTGCTCATTTCATGGGGG |
| 1891 | CGAATTGATTCATTAACCAGGGG |
| 1892 | GCACAGTTCGAGGTCCCAGAGGG |
| 1893 | GCACGATTGAGGATGCACATTGG |
| 1894 | TCCATAGATGACATACTGGAAGG |
| 1895 | GGTTATCCATGCTTTGAGTGAGG |
| 1896 | AACAAGGCTTCTTTGTCCGGGGG |
| 1897 | CGTAGAATCTGTGTAGACGAAGG |

TABLE 92
Target sequences for PMP22 gene

| SEQ ID NOS | Target sequence |
|---|---|
| 1898 | GCGCGTAAAGCTTCACACAGAGG |
| 1899 | CAGGATGTAGGCGAAACCGTAGG |
| 1900 | TGTCAGGAGCGAAATCATTGCGG |
| 1901 | TATAAATCCAGTATGCCGTGTGG |
| 1902 | CTTCTTTAAGGCTCAACACGAGG |
| 1903 | GCCAGGTTTTCCCAAAACGTGGG |
| 1904 | TCCGACCGTAAGAAAAATGTGGG |
| 1905 | ACACACAACAAAAGGTCGACGGG |
| 1906 | ACAGACAGCGTCCCCCCACAAGG |
| 1907 | TGTCACACGATAAGGGAACCAGG |

TABLE 93
Target sequences for PMS2 gene

| SEQ ID NOS | Target sequence |
|---|---|
| 1908 | CTCCTGTGTCTACGGTGAGC |
| 1909 | ACTAGTAAAAACTGGACCTT |
| 1910 | AAGGTCCAGTTTTTACTAGT |
| 1911 | TCTTTTTGACGAGCATAGAT |
| 1912 | CTATGCTCGTCAAAAAGACG |
| 1913 | TCGTCAAAAAGACGTGGATG |
| 1914 | GTGGTGCATTGGTTGACTGT |
| 1915 | TTAGACTTCATTGACAAACC |
| 1916 | TGAGATATAAGCGTCCTACC |
| 1917 | GACGCTTATATCTCATGTCT |
| 1918 | AGGATCACTATTGCAGTTCA |
| 1919 | GGATCACTATTGCAGTTCAC |
| 1920 | ACAGTCAACCAATGCACCAC |
| 1921 | GAGACCCACCCCAGGGATAC |
| 1922 | AGGATGGTCAAAGTGCAACG |
| 1923 | CCAATAAAGAGAACGGGGAC |
| 1924 | GTCCTCAAGTTAGAGAAGTC |

TABLE 94
Target sequences for PRSS1 gene

| SEQ ID NOS | Target sequence |
|---|---|
| 1925 | TAGTAAGTTATGTGCTATATAGG |
| 1926 | GCCCITTCCCGCAAGGATGCTGG |
| 1927 | AACGCCCTGCAGGCTTGTTAAGG |
| 1928 | CGGGGTTGGCACATGACATATGG |
| 1929 | TGACCTTGCCCGACACTGACTGG |
| 1930 | CCATAAACTAATCGACAGTCAGG |
| 1931 | CACGGTTCCACGTGAGTACATGG |
| 1932 | GTATCTACAGTTGTTAGAGCAGG |
| 1933 | AGAGGCACGTCATCACCAACAGG |
| 1934 | TCTTCCTGTCGTATTGGGGGTGG |
| 1935 | GAGTCTTCCTGTCGTATTGGGGG |
| 1936 | GGCGTTGATTACTGCACGTGAGG |
| 1937 | AAGAGTCTTAGTGGCCCAGGTGG |
| 1938 | TAGGAGCTTAGTGCATCTGGAGG |

TABLE 95
Target sequences for PTCH1 gene

| SEQ ID NOS | Target sequence |
|---|---|
| 1939 | ATTTCAAAAGCGTCTCTGCGCGG |
| 1940 | TTGAAAGAGCACTAATGACGGGG |
| 1941 | GGAGGTCTATAATTACCAAGAGG |
| 1942 | CGAGGAGCTTCGGCACTACGAGG |
| 1943 | CCCATGTGACCAATTCGCTGTGG |
| 1944 | TAAGAGATGCCGTAGACACGAGG |
| 1945 | AGTGCCGAAGCTCCTCGCTGAGG |
| 1946 | GAAGCACGTACCCTAAACACTGG |
| 1947 | GTCCAATTATGCATCTCAAGGGG |
| 1948 | TATTACTGCTACCCAAGATGGGG |

TABLE 96
Target sequences for PTEN gene

| SEQ ID NOS | Target sequence |
|---|---|
| 1949 | GTAGTCCCGGAGTTAGGTAA |
| 1950 | CCAGGTTTAATTAGTAGTCC |
| 1951 | CCTATGGAAGAACGTATATG |
| 1952 | AGGTTAGACTAACCTTAAAT |
| 1953 | CCACATATACGTTCTTCCAT |
| 1954 | CATATACGTTCTTCCATAGG |
| 1955 | ATTTAAGTTGCCCAACCAAC |
| 1956 | TAGCGAGAGCAAAACTGTAG |
| 1957 | GGTTATAGCTACCAATACTC |
| 1958 | ATTGGTAGCTATAACCACTT |
| 1959 | TTGGTAGCTATAACCACTTT |
| 1960 | GGTATGAGTACTAATCTGGC |
| 1961 | GTATGAGTACTAATCTGGCT |
| 1962 | GGTGAAGTTATTGCAATCTA |
| 1963 | TTTTGGTATGAGTACTAATC |
| 1964 | TGGTGTGCTAGTTTTTACGT |
| 1965 | CCCGATTAATATTTAGCCAG |
| 1966 | CAATGGTTGGTACTAACAGG |
| 1967 | GAACAATGGTTGGTACTAAC |
| 1968 | ACGTGATATCTTTTTGTAAC |
| 1969 | AGTTTAAACCATAGACGCAA |
| 1970 | GGGAACATACTACCACTGTT |
| 1971 | CTTTGTAGGAGAGGTTTATC |
| 1972 | TCCTACAAAGAGCCTTGTTG |
| 1973 | CGGATACCATAGTGTTTCTT |
| 1974 | AGGGTTAGACTATCAGAACT |
| 1975 | GGGTTAGACTATCAGAACTG |
| 1976 | CCATTAAACTGAGTCACTTC |
| 1977 | CCTATTTCACAACACCCTAC |
| 1978 | GGGATATTCCAACCTATGCA |
| 1979 | GATGAAATCGTAAGTCCTGT |
| 1980 | ATGAAATCGTAAGTCCTGTA |
| 1981 | CTATCACTCAATAACTCTTC |
| 1982 | GCCCTACCCACAACATAAAC |
| 1983 | AATTCATTTGTCATACGCTG |
| 1984 | TGGCACTTCTTAACCTCCTA |
| 1985 | GTAGTAGGTGTTTACTAAAC |
| 1986 | GCTCATATTACAACGTACAA |

TABLE 97
Target sequences for REEP1 gene

| SEQ ID NOS | Target sequence |
|---|---|
| 1987 | CATCTGGTCCAATCACCGTGAGG |
| 1988 | TCCCCATATAAGTCTCACAAGGG |
| 1989 | ATTGGCGTTTTCTGACGACGAGG |
| 1990 | GGCGTTTTCTGACGACGAGGAGG |
| 1991 | TGATCTGTGTATCCCATGGAAGG |
| 1992 | GTTGGCTCATCTCACTCACGTGG |
| 1993 | AGGCAGATTACTATAAAGGTGGG |
| 1994 | CACTTAACATCTAACACACCAGG |
| 1995 | CAGATGTTAATTAAGCTGGATGG |
| 1996 | GGTTTTAGAAGATTGCGAGTTGG |

TABLE 98
Target sequences for RPGR gene

| SEQ ID NOS | Target sequence |
|---|---|
| 1997 | TCGCTTGTCAGAGATCCCAGAGG |
| 1998 | ATATTGACCCTACGACAACAAGG |
| 1999 | AGGTTTCTCTCAGAACATCGTGG |
| 2000 | TGCCAACTCAGTAAACCGAAGGG |
| 2001 | AATGGCACCAAGTAACCAGTGGG |
| 2002 | GGTAGCAACTAATAATGACCAGG |
| 2003 | GTTCTTAACGAGCAAACCAGAGG |
| 2004 | AATACAGGTATGATGCGTGATGG |
| 2005 | GGACTCTATCAGCACGTATGCGG |
| 2006 | TAAACTAATTCGTACCAGAAAGG |

TABLE 99
Target sequences for SBDS gene

| SEQ ID NOS | Target sequence |
|---|---|
| 2007 | TGGCGAAAGTAAATACGCCAAGG |
| 2008 | GCTGTATCAAATGGTGCACATGG |
| 2009 | GCGGTACCAGTGCGAATCATCGG |
| 2010 | CGGTACCAGTGCGAATCATCGGG |
| 2011 | ATCCTGGTGGTATCTTGTCGTGG |
| 2012 | TGCGAATCATCGGGCTATCCAGG |
| 2013 | CACTCGGTACGCCGCTAACGCGG |

TABLE 100
Target sequences for SCNIA gene

| SEQ ID NOS | Target sequence |
|---|---|
| 2014 | TCCCGATGCAACTCAGTTCATGG |
| 2015 | GACCCTAATAAAGTTAACCCTGG |
| 2016 | AAACTTGTACCTATACTGTTGGG |
| 2017 | CATTTTGICACGCATCAATCTGG |
| 2018 | TGATATGTGTTGCATACCTCTGG |
| 2019 | ATGGTTGCCAAGTAATATCAGGG |
| 2020 | ACTCACTAAGCATAAGGTCTTGG |
| 2021 | TTCGCCACTCACTAAGCATAAGG |
| 2022 | GCTTAGTGAGTGGCGAAATTTGG |
| 2023 | ATCAGAAGTTATCCCATTATAGG |
| 2024 | TGAACAATTGAATTGCTCCGTGG |
| 2025 | TTTATATAGTTCGAGTGTCTGGG |
| 2026 | GGTAGTATAAAAAGTCTGCAGGG |
| 2027 | GTCAGTCCATTGTACAAGGATGG |
| 2028 | CCAATTGGGAGCTCAAAGGTAGG |
| 2029 | TCCAGTGACATATCTACCCTAGG |
| 2030 | ACCTTCAATTCAGTTAGTGCAGG |
| 2031 | TGGGGGGTATGGCAACCACATGG |
| 2032 | TTTGTCACCCGGTCATAGGAAGG |
| 2033 | GTCACCCGGTCATAGGAAGGTGG |

TABLE 101
Target sequences for SDHB gene

| SEQ ID NOS | Target sequence |
|---|---|
| 2034 | GCGTCTCTGGGAAGAAACCGGGG |
| 2035 | TGAAATTTCCAGTCCCACGTGGG |
| 2036 | TTTGGTACAGGAACACACGTTGG |
| 2037 | GTGTGTATAATTAAGCACCCGGG |
| 2038 | GGCCGATCATGAAACTGGAAGGG |
| 2039 | CTCTCGGTGTGTGGTCATCGAGG |
| 2040 | GCCACTGCCAATCCTGACGGAGG |
| 2041 | GGTCCCCACAGGGTCAGTAAGGG |
| 2042 | CTTCCAATCCCGCGGCTGAGGGG |
| 2043 | CGAGTTAATCACATGACCATAGG |

TABLE 102
Target sequences for SDHC gene

| SEQ ID NOS | Target sequence |
|---|---|
| 2044 | TATTATCACTGGTCTCCCCGAGG |
| 2045 | ATACATTCACCACATCGCGGTGG |
| 2046 | TAGTATCTCACCTTGGAACGGGG |
| 2047 | GTGGTTCCATCAATATCCTGAGG |
| 2048 | AAACAACGCACTTCACAACGTGG |
| 2049 | ATTGTGGGGTCTAATCGAGGTGG |
| 2050 | ATTGTCTGACCAACGCTGGGGGG |
| 2051 | TGGTCCGCAAGGTCTTCTCGAGG |
| 2052 | GTGCGCTCCGTAGGGCTTCGGGG |
| 2053 | TTGATGGATATGTACGACAGTGG |

TABLE 103
Target sequences for SDHD gene

| SEQ ID NOS | Target sequence |
|---|---|
| 2054 | CTGAGCACTACCGGTCACCAGGG |
| 2055 | AAAACTCTGAATCGGTCGAGGGG |
| 2056 | TAGGTGGGTTAATAAGCTAGAGG |
| 2057 | GCGTTAGAACCATGTCCGAAGGG |
| 2058 | TAGCATTACTACAGTACCTGAGG |
| 2059 | TATTCCCAGCAGAACCACGAGGG |
| 2060 | GCTGGATCCAATAGTGACCTGGG |
| 2061 | GAGATTCCTTGAACATGCCAGGG |
| 2062 | GTTCGAAAATCATTTAACCTGGG |
| 2063 | GCGATGGAGAGAACATACAATGG |

TABLE 104
Target sequences for SHOX gene

| SEQ ID NOS | Target sequence |
|---|---|
| 2064 | TTGGCAACGAAAAACGTGTGGGG |
| 2065 | CCCAAGATCGTGCGTCCCCGGGG |
| 2066 | AATCAATAAACAGCGTCGGAGGG |
| 2067 | CGTCAATCAATAAACAGCGTCGG |
| 2068 | AACCCCTGCGCTCACCCGCGGGG |
| 2069 | TTGCAGCTCCCGTCTCGCCAGGG |
| 2070 | ATTGGCAACGAAAAACGTGTGGG |
| 2071 | CGGCGCATCTTCCTCCCCGGCGG |
| 2072 | GCTTTTTCTCCGAGGCCGAGGGG |
| 2073 | CCAAATCACCTGGCCACACGGGG |

TABLE 105
Target sequences for SLC6A4 gene

| SEQ ID NOS | Target sequence |
|---|---|
| 2074 | GTAGTAAAAAGGGGCAAACGTGG |
| 2075 | CTACTGCACCCATAAATATGAGG |
| 2076 | TAAGGGGAGTTGCTTTACAGTGG |
| 2077 | GAACGTATTTGTGAACCGATAGG |
| 2078 | AGGAGCTCGTAGAATTGTCATGG |
| 2079 | TGAAAGTTCTGCCCCCGAGAGGG |
| 2080 | CTTTCCAGCAACAGCACGAGCGG |
| 2081 | GTGCAGGCCACGAGACCCGAAGG |
| 2082 | CACGCTGCAAGGTAAGATGTTGG |
| 2083 | TGAGAGCGCTATAAAGGCAGCGG |

TABLE 106
Target sequences for SLC6A5 gene

| SEQ ID NOS | Target sequence |
|---|---|
| 2084 | CTTGCTTAACCTCCGCACTGCGG |
| 2085 | GTTTAGGCAGAAACACTCGTAGG |
| 2086 | GCTACCCCCATACAACCGAGTGG |
| 2087 | TACCTCTTCTGTACCCACCGAGG |
| 2088 | ATTCAGACCGAATGGCTGCGCGG |
| 2089 | CGGTCCGGTTGAGAAGATGTGGG |
| 2090 | ACGCGCGGCAGTCTCCACGCCGG |
| 2091 | CGAGTTGCTCTGGGTCCTAGAGG |
| 2092 | AGGCTTGAGTGCATAACCAGAGG |
| 2093 | GGGATCTGCGAAGAGCGGCGGGG |

TABLE 107
Target sequences for SLC6A8 gene

| SEQ ID NOS | Target sequence |
|---|---|
| 2094 | ACTCTCCAAGCACATTACAGGGG |
| 2095 | ATAGGTCTATGTGGTCCGGGTGG |
| 2096 | GGTCTGATCAGGTCTTGAAGGGG |
| 2097 | GATGAGGCGCTTCACCCCCGTGG |
| 2098 | AGGCAAGCGAGTCCTCTACCCGG |
| 2099 | CGCGTCCAGGTCTCGCGCGGCGG |
| 2100 | GGTCATCCTGCAAACCTTCGGGG |
| 2101 | ACCGCAGCATTCTGGTCCGTAGG |
| 2102 | GCAGACAAACGAGGCGCCCAGGG |
| 2103 | CCGCAGCATTCTGGTCCGTAGGG |

TABLE 108
Target sequences for SLC22A5 gene

| SEQ ID NOS | Target sequence |
|---|---|
| 2104 | GCTAATTCCCCAGTACCCCAGGG |
| 2105 | TGGCTTGGGAACGCTTCACGAGG |
| 2106 | GCATCCAACCCCTAATCAGGAGG |
| 2107 | GGGCCATAGAGCATCGCCCAGGG |
| 2108 | GAGTTGTCAAGGGCGGTCAGTGG |
| 2109 | GTCCCTCTTATAAGATTAGGCGG |
| 2110 | GTATTATAGAAGGGTTTTCGGGG |
| 2111 | TGAGGTAAGGGATGTGCTCGGGG |
| 2112 | GTGGGTAAGTATCCCTGCAGTGG |
| 2113 | TTACATAGGGCGCACGACCAGGG |

TABLE 109
Target sequences for SMAD4 gene

| SEQ ID NOS | Target sequence |
|---|---|
| 2114 | CTTCGGGAAGAAACAGACGCTGG |
| 2115 | TCTTATAACCACCTACCACTAGG |
| 2116 | TAGTAGAATCATTACATGCGAGG |
| 2117 | GTCATACCAAAAGGCCACATTGG |
| 2118 | TGAGTGGCGAAGGCGTACGGTGG |
| 2119 | ATTCTCCCACGAGCTGCAAGCGG |
| 2120 | GTTTGAGGGAGTGGTCGCCGGGG |
| 2121 | GCAATTCAACCATGTGAGGGTGG |
| 2122 | TTAATGGGGTAAGCTAAGCCAGG |
| 2123 | TTTTGCACCGTAGTTTAAGGTGG |

TABLE 110
Target sequences for SMARCE1 gene

| SEQ ID NOS | Target sequence |
|---|---|
| 2124 | GTAGTGCTATGGATTAAACGAGG |
| 2125 | GTGTAGGAATCATATCACCTGGG |
| 2126 | CAGAACCATGACGACCTTAGGGG |
| 2127 | AGCTATTGTCCCAGAATACGTGG |
| 2128 | GCATCGTTGCAAGAAGTGGGAGG |
| 2129 | AAGTAACTACTCTAACTATGGGG |
| 2130 | CAGTGAGGGCCATAGTTCGTTGG |
| 2131 | GTGCCTATACCACAAATCCCAGG |
| 2132 | GGTAGATTTAGGCATGGTGTAGG |
| 2133 | AGTCCTCTCCATATAGGCACAGG |

TABLE 111
Target sequences for SMN1 gene

| SEQ ID NOS | Target sequence |
|---|---|
| 2134 | CCGGGTGTAAGGGGGCCATTAGG |
| 2135 | TTCAAATAATGTCGGGGTGGTGG |
| 2136 | CTTCATATCACTGTACCTACTGG |
| 2137 | GCCGAGTTCCGGGTGTAAGGGGG |
| 2138 | ACACACTGGAGTTCGAGACGAGG |
| 2139 | GAAGGATGGCCAGCTCTTATTGG |
| 2140 | AAGGATGGCCAGCTCTTATTGGG |
| 2141 | TACATGAGTGGCTATCATACTGG |
| 2142 | GTTGTTGCGCAATAGATCTTCGG |
| 2143 | CATATCTTATACAGGTGACATGG |
| 2144 | TCATCTCGTTTTGATCAGTGGGG |
| 2145 | GGTGTAGATTAGTAATGAAGTGG |
| 2146 | GCATGGCAGCGCACTGTTAAAGG |
| 2147 | GCAGTCCTGGTGGTCCGTTCTGG |
| 2148 | CACATCTATGATACGTGAATGGG |
| 2149 | TCATACACAATCTTGCTGTCTGG |
| 2150 | AAACCCGCGGGTGCGCAGCGTGG |
| 2151 | ACGAATCTGCCAAAACTTAGTGG |
| 2152 | CTTCTCACGCTTTCTACGAGTGG |
| 2153 | GCGTTTGGAGCATATTGTGTAGG |
| 2154 | AGTTTCAAATAATGTCGGGGTGG |
| 2155 | CGCACGAAAACTGCCCAGCACGG |
| 2156 | CGTGCTGGGCAGTTTTCGTGCGG |
| 2157 | TGCCGCACCCAGCTGTAAACTGG |
| 2158 | CTATAGGGTAGAGTTGGATTTGG |
| 2159 | CAGGAAACTTACCTGGTTAGAGG |
| 2160 | TTCCCTGGTCATATCTTGGTTGG |
| 2161 | ATCATCTCGTTTTGATCAGTGGG |
| 2162 | AAGTTGGTGTCTATGCCATAAGG |
| 2163 | ATATCTTATACAGGTGACATGGG |
| 2164 | GTGTAGATTAGTAATGAAGTGGG |
| 2165 | AGAGCTCAATTCATTAAGCGTGG |
| 2166 | ACATCGGTAGGCATATTTCAAGG |
| 2167 | GATTCGTGGTCATGAGTTGAAGG |
| 2168 | CGTCACTCTTAAGAAGGGACGGG |
| 2169 | GCTATGGCGATGAGCAGCGGCGG |
| 2170 | GAGCCCAAACTGCTCGAGGAAGG |
| 2171 | GATTCCGTGCTGTTCCGGCGCGG |
| 2172 | CCGCTATTCACAACAAATATGGG |
| 2173 | TCTACTCATGGTATGTGGATAGG |
| 2174 | TAGGCATTCCCAATAAGAGCTGG |
| 2175 | GATTGAAATGGGGCTCGATGTGG |
| 2176 | CAGAAGTAATGAAACCGTTGGGG |
| 2177 | CACGTTACTAAGAGCAACTCTGG |
| 2178 | AACCCGCGGGTGCGCAGCGTGGG |
| 2179 | GGCCGAGTTCCGGGTGTAAGGGG |
| 2180 | GTTACTACAAGCGGTCCTCCCGG |
| 2181 | GTTTTCGTGCGGCTGTCTCGTGG |
| 2182 | CCCGCTATTCACAACAAATATGG |
| 2183 | GAAGCGTTATAGAAGATAACTGG |
| 2184 | CGTGAGCTTAGAGCATAGACTGG |
| 2185 | TAGGCCGAGTTCCGGGTGTAAGG |
| 2186 | GAACTGCGATGTAAACATTAAGG |
| 2187 | TGTCTTTATATAGATCAAGCAGG |
| 2188 | CGATAGTTAGACAGAGTCCTCGG |
| 2189 | TCAGATAGATTCGATAACGGAGG |
| 2190 | CTTAAGGTTACATTCGCACTTGG |
| 2191 | ATAGCAATGTAGGGCCCCAACGG |
| 2192 | AATAAGGTATAAGCGGGCTCAGG |
| 2193 | AGGCCGAGTTCCGGGTGTAAGGG |
| 2194 | CATCAAGTCGATCCGCTCACTGG |
| 2195 | CGATCCGCTCACTGGAGTTGTGG |
| 2196 | AGGTTACATTCGCACTTGGAAGG |
| 2197 | GGTTACATTCGCACTTGGAAGGG |
| 2198 | GTTGTCAGTTTGATCCACCGAGG |

TABLE 112
Target sequences for SMN2 gene

| SEQ ID NOS | Target sequence |
|---|---|
| 2199 | TCATCTCGTTTTGATCAGTGGGG |
| 2200 | GTTGTCAGTTTGATCCACCGAGG |
| 2201 | GATTCCGTGCTGTTCCGGCGCGG |
| 2202 | ATATCTTATACAGGTGACATGGG |
| 2203 | ACACACTGGAGTTCGAGACGAGG |
| 2204 | CAGAAGTAATGAAACCGTTGGGG |
| 2205 | CGTGCTGGGCAGTTTTCGTGCGG |
| 2206 | ATAGCAATGTAGGGCCCCAACGG |
| 2207 | AGAGCTCAATTCATTAAGCGTGG |
| 2208 | TCAGATAGATTCGATAACGGAGG |
| 2209 | CGCACGAAAACTGCCCAGCACGG |
| 2210 | AGTTTCAAATAATGTCGGGGTGG |
| 2211 | GTGTAGATTAGTAATGAAGTGGG |
| 2212 | GAGCCCAAACTGCTCGAGGAAGG |
| 2213 | GCCGAGTTCCGGGTGTAAGGGGG |
| 2214 | GGTTACATTCGCACTTGGAAGGG |
| 2215 | GGTGTAGATTAGTAATGAAGTGG |
| 2216 | GCGTTTGGAGCATATTGTGTAGG |
| 2217 | CGATAGTTAGACAGAGTCCTCGG |
| 2218 | GTTTTCGTGCGGCTGTCTCGTGG |
| 2219 | GATTGAAATGGGGCTCGATGTGG |
| 2220 | TGTCTTTATATAGATCAAGCAGG |
| 2221 | GCTATGGCGATGAGCAGCGGCGG |
| 2222 | CGATCCGCTCACTGGAGTTGTGG |
| 2223 | TAGGCATTCCCAATAAGAGCTGG |
| 2224 | GTTACTACAAGCGGTCCTCCCGG |
| 2225 | AGGTTACATTCGCACTTGGAAGG |
| 2226 | CATATCTTATACAGGTGACATGG |
| 2227 | CGTCACTCTTAAGAAGGGACGGG |
| 2228 | CATCAAGTCGATCCGCTCACTGG |
| 2229 | CTTCTCACGCTTTCTACGAGTGG |
| 2230 | CGTGAGCTTAGAGCATAGACTGG |
| 2231 | AAGTTGGTGTCTATGCCATAAGG |
| 2232 | CTTCATATCACTGTACCTACTGG |
| 2233 | GTTGTTGCGCAATAGATCTTCGG |
| 2234 | ATCATCTCGTTTTGATCAGTGGG |
| 2235 | GATTCGTGGTCATGAGTTGAAGG |
| 2236 | ACGAATCTGCCAAAACTTAGTGG |
| 2237 | GGCCGAGTTCCGGGTGTAAGGGG |
| 2238 | TACATGAGTGGCTATCATACTGG |
| 2239 | CTTAAGGTTACATTCGCACTTGG |
| 2240 | AAACCCGCGGGTGCGCAGCGTGG |
| 2241 | ACATCGGTAGGCATATTTCAAGG |
| 2242 | TCATACACAATCTTGCTGTCTGG |
| 2243 | AATAAGGTATAAGCGGGCTCAGG |
| 2244 | TTCAAATAATGTCGGGGTGGTGG |
| 2245 | TCTACTCATGGTATGTGGATAGG |
| 2246 | GAACTGCGATGTAAACATTAAGG |
| 2247 | CACATCTATGATACGTGAATGGG |
| 2248 | TTCCCTGGTCATATCTTGGTTGG |
| 2249 | CAGGAAACTTACCTGGTTAGAGG |
| 2250 | AGGCCGAGTTCCGGGTGTAAGGG |
| 2251 | CTATAGGGTAGAGTTGGATTTGG |
| 2252 | GAAGCGTTATAGAAGATAACTGG |
| 2253 | TAGGCCGAGTTCCGGGTGTAAGG |
| 2254 | CACGTTACTAAGAGCAACTCTGG |
| 2255 | CCGCTATTCACAACAAATATGGG |
| 2256 | TGCCGCACCCAGCTGTAAACTGG |
| 2257 | CCGGGTGTAAGGGGGCCATTAGG |
| 2258 | GCAGTCCTGGTGGTCCGTTCTGG |
| 2259 | AAGGATGGCCAGCTCTTATTGGG |
| 2260 | AACCCGCGGGTGCGCAGCGTGGG |
| 2261 | GCATGGCAGCGCACTGTTAAAGG |
| 2262 | GAAGGATGGCCAGCTCTTATTGG |
| 2263 | CCCGCTATTCACAACAAATATGG |

TABLE 113
Target sequences for STK11 gene

| SEQ ID NOS | Target sequence |
|---|---|
| 2264 | GGACTCTTCTGTCAATTTCG |
| 2265 | ACACCCAGGCCTATTTGTCG |
| 2266 | GGGCACAAACAGAGGCCTCG |
| 2267 | GCGAAAATCCTCTTTACCAT |
| 2268 | CAGATGCTGGAACCCCATAA |
| 2269 | TGCTTGGACCTATGGTAAAG |
| 2270 | TTGGCAGATGCTTGGACCTA |
| 2271 | GTAGGTCTTTACATCCCAGG |
| 2272 | GATACCTGGACGCTCCTAAG |
| 2273 | ATACCTGGACGCTCCTAAGG |
| 2274 | GTGATACCTGGACGCTCCTA |
| 2275 | TGATACCTGGACGCTCCTAA |
| 2276 | TCCACTCCTGGGACATGCCG |
| 2277 | GAGCGTCCAGGTATCACCCA |
| 2278 | GGAGCGTCCAGGTATCACCC |
| 2279 | AAGCCCAGGGCCCACGTCGG |
| 2280 | CGGCTCCCACGTCCACTGGG |
| 2281 | CACGTCGGTGGGATGGGAAT |

TABLE 114
Target sequences for TGFBR1 gene

| SEQ ID NOS | Target sequence |
|---|---|
| 2282 | TGGGTTTTTAGTGACACCTCAGG |
| 2283 | TTTCCAACCTGGATCGGGAAGGG |
| 2284 | TCACAACGATCAGGTAAATTAGG |
| 2285 | ACACTATCTTCACAACGATCAGG |
| 2286 | GTCATGGTTGCTGATGTTACAGG |
| 2287 | GTGTCAGCTTTACTATCTCCTGG |
| 2288 | CTGAAGTCCTAGCTTGTATCTGG |
| 2289 | TTTTATTCGTAGGCCACCAAAGG |
| 2290 | GTAGTAGAAAGGTCCTAAACAGG |
| 2291 | GTAGGAGTCTAAACCAAATCAGG |
| 2292 | CATGTCTTAACCTTTCAGTCTGG |
| 2293 | TAAACCAAATCAGGTCCACCTGG |
| 2294 | GTCTTAACCTTTCAGTCTGGAGG |
| 2295 | AGTTGCGTAGGTTTCACTCGTGG |
| 2296 | CCTTCCCCACTTATCACATCAGG |
| 2297 | AGTGACCTGATGTGATAAGTGGG |
| 2298 | TCAATAAGTCAGCTCCATGGTGG |
| 2299 | AGTGATACCTCTAACACATGGGG |
| 2300 | TAGGTTAAATTAGATTGTCGTGG |
| 2301 | ACTATGTTCTGATACACTAAAGG |
| 2302 | AACACTGTAATAGGTCTCTCAGG |
| 2303 | GATGCTTCAGTGGTTACTCCAGG |
| 2304 | AAGAGTGTGCATTCTGTTCGTGG |
| 2305 | AGTGTGCATTCTGTTCGTGGTGG |

TABLE 115
Target sequences for TGFBR2 gene

| SEQ ID NOS | Target sequence |
|---|---|
| 2306 | CACCACTATCACTTCGTGATAGG |
| 2307 | TACCCCGTTTGCACATGAGAGGG |
| 2308 | TTCCATTGAGATCACAAGACAGG |
| 2309 | TTCCAACACCCATGCTATAATGG |
| 2310 | ACTACTTGTCCATTATAGCATGG |
| 2311 | GTCCATTATAGCATGGGTGTTGG |
| 2312 | CATGGGTGTTGGAAGACTAGAGG |
| 2313 | ACAGTCCTAATCAAGCCCACTGG |
| 2314 | GGATTCCATAGCAAGTCTTCTGG |
| 2315 | TGAGATACAGGCCACATAACAGG |
| 2316 | TTGTTAGAAACCAAGCGCCTTGG |
| 2317 | TCCCAAATATGGTAGTACTCTGG |
| 2318 | TATACAACTTATGCTGCTGAGGG |
| 2319 | ATAGAAATTCTTCTCCGTGCTGG |
| 2320 | AACCCAGACCTATAGTTAGTTGG |
| 2321 | TTTCCAACTAACTATAGGTCTGG |
| 2322 | TCACTATTCTCACGTTTCTAAGG |
| 2323 | TTCCAACTAACTATAGGTCTGGG |
| 2324 | CAATGCTAGTAAACATGCCTGGG |
| 2325 | TTGATAAATGGCCTGCAAGTTGG |
| 2326 | TCTCTGACAGTAGAATACCCAGG |
| 2327 | TCTAGTCAATTAACTGGTGGAGG |
| 2328 | CGGGCACACTTAGAATAACGAGG |
| 2329 | CTTGCCATCCCCCACGGACAGGG |
| 2330 | ACTGAGTGTTATCTAAGCTCAGG |
| 2331 | ACGGACAGGGAACTCCATGCTGG |
| 2332 | GTTGTACTGAATTGTTACCTAGG |
| 2333 | ATGGAGTTCCCTGTCCGTGGGGG |
| 2334 | AGCAACTTGACAATACACTAAGG |
| 2335 | ACGTGTCAGCTTCTATTCAAAGG |
| 2336 | GGGACAGCAATGGTATTCCTCGG |
| 2337 | GTGTTACTGTTCTACGAAAAAGG |
| 2338 | ACGGGTAGTCTGAAAGGTGCTGG |
| 2339 | GAGGTCACGGGTAGTCTGAAAGG |
| 2340 | GTTACATGAGGTCTCATCCTAGG |
| 2341 | AGGTTGAAATACCCTGGTGCAGG |

TABLE 116
Target sequences for VHL gene

| SEQ ID NOS | Target sequence |
|---|---|
| 2342 | ACCATAGGTGGTACATAGTAGGG |
| 2343 | CACCATAGGTGGTACATAGTAGG |
| 2344 | TATTGAAGTGCAGTGAAGGCAGG |
| 2345 | TCAACACTTATCACCATAGGTGG |
| 2346 | TAGTAATTTCACCTTGAAATGGG |
| 2347 | GGCCCCCTATGGACACCTCATGG |
| 2348 | ATTTCACCTTGAAATGGGCTGGG |
| 2349 | CAGTACAAGGAACGAACAAGAGG |
| 2350 | CTCAGGCGATCTACTGACGTTGG |
| 2351 | GTATAAAAGCAGAAGTCAGCAGG |
| 2352 | CACCATGAGGTGTCCATAGGGGG |
| 2353 | TCAAGGTGAAATTACTACAGAGG |
| 2354 | TCTAGCCCATGCCCTCACTGTGG |
| 2355 | GCCAATGACTAGCAGAGCGTGGG |
| 2356 | ACTAGCAGAGCGTGGGACTGAGG |

TABLE 117
Target sequences for WT1 gene

| SEQ ID NOS | Target sequence |
|---|---|
| 2357 | AATCTTGTCTAACATTCCCGAGG |
| 2358 | GTTCCCAACTTACTCAACAAGGG |
| 2359 | TGGTATGGTTTCTCACCTTGGGG |
| 2360 | TTGATCGTCCTAACTGTACAGGG |
| 2361 | TGTAGCGAGGATCTACAGGGTGG |
| 2362 | GAATGCTACTAACACTGGTGGGG |
| 2363 | GTCCTGAGCTCATAATTCGGTGG |
| 2364 | GTAGCGAGGATCTACAGGGTGGG |
| 2365 | TACTCCTTACAACTGCCCGTAGG |
| 2366 | CTCCTTACAACTGCCCGTAGGGG |

### Sequencing

Following target enrichment of the sample using the methods and systems described elsewhere, the highly fragmented gDNA samples can be sequenced to detect genomic variations. In some embodiments, short-read sequencing is used. In some embodiments, long-read sequencing. In some cases, the sample contains high fragmented RNA samples. In some case the sample contains full-length RNA transcripts.

In some embodiments, the long-read sequencing platform may be single molecule real time sequencing (SMRT) (e.g. Pacific Biosciences long-read sequencing technology), or a variation thereof. Single-molecule real-time sequencing (SMRT) is a parallelized single molecule DNA sequencing method. Single-molecule real-time sequencing utilizes a zero-mode waveguide (ZMW). A single DNA polymerase enzyme is affixed at the bottom of a ZMW with a single molecule of DNA as a template. The ZMW is a structure that creates an illuminated observation volume that is small enough to observe only a single nucleotide of DNA being incorporated by DNA polymerase. Each of the four DNA bases is attached to one of four different fluorescent dyes. When a nucleotide is incorporated by the DNA polymerase, the fluorescent tag is cleaved off and diffuses out of the observation area of the ZMW where its fluorescence is no longer observable. A detector detects the fluorescent signal of the nucleotide incorporation, and the base call is made according to the corresponding fluorescence of the dye.

In other embodiments, the long-read sequencing platform may be nanopore sequencing (e.g. Oxford Nanopore long-read sequencing technology), or a variation thereof. Nanopore sequencing uses electrophoresis to transport an unknown sample through an orifice of about 10⁻⁹ meters in diameter. A nanopore system can contains an electrolytic solution; when a constant electric field is applied, an electric current can be observed in the system. The magnitude of the electric current density across a nanopore surface depends on the nanopore's dimensions and the composition of DNA or RNA molecule that is occupying the nanopore. Sequencing is made possible because, while traversing through the nanopore, samples cause characteristic changes in electric current density across nanopore surfaces. The total charge flowing through a nanopore channel is equal to the surface integral of electric current density flux across the nanopore unit normal surfaces between times t₁ and t₂.

In some cases, long-read sequencing requires application of the sample. In other cases, long-read sequencing does not require application of the sample.

### Clinical Applications

The systems and methods described herein can be used in clinical settings to detect and diagnose genetic diseases or disorders. In some embodiments, the systems and methods described herein can be used can be used in the detection, treatment and/or monitoring of hereditary breast cancer-related disorders by detecting genetic variations in relevant genes such as BRCA1, BRCA2, MLH1, MSH2, and STK11. In some embodiments, the systems and methods described herein can be used can be used in the detection, treatment and/or monitoring of hereditary colon cancer-related disorders by detecting genetic variations in relevant genes such as MLH1, MSH2, EPCAM, SMAD4, and STK11. In some embodiments, the systems and methods described herein can be used can be used in the detection, treatment and/or monitoring of hereditary neuroendocrine tumor disorders by detecting genetic variations in relevant genes such as SDHB, SHDC, SDHD, and VHL. In some embodiments, the systems and methods described herein can be used can be used in the detection, treatment and/or monitoring of Cowden Syndrome by detecting genetic variations in relevant genes such as PTEN.

In some embodiments, the systems and methods described herein can be used can be used in the detection, treatment and/or monitoring of neuromuscular disorders such as Duchenne Muscular Dystrophy and Spinal Muscular Atrophy by detecting genetic variations in relevant genes such as DMD, SMN1, and SMN2.

In some embodiments, the systems and methods described herein can be used can be used in the detection, treatment and/or monitoring of Fragile X Syndrome by detecting genetic variations in relevant genes such as FMR1.

In some embodiments, the systems and methods described herein can be used can be used in the detection, treatment and/or monitoring of cardiovascular disorders such as aortic dysfunction and dilation, and cardiac ion channelopathies, by detecting genetic variations in relevant genes such as TGFBR1, TFRBR2, MYH11, COL3A1, KCNH2 and KCNQ1.

In some embodiments, the systems and methods described herein can be used can be used in the detection, treatment and/or monitoring of movement disorders such as Parkinson Disease, Hereditary Ataxia, and Dystonia 5, by detecting genetic variations in relevant genes such as SCNA, PARK2, PARK7, PINK1, SCA1 (ATXN1), SCA10 (ATXN10), SCA17 (TBP), SCA2 (ATXN2), SCA3 (MJD/ATXN3), SCA6 (CACNA1A), SCAT (ATXN7), SCAB (ATXN8OS) and GCH1.

In some embodiments, the systems and methods described herein can be used can be used in the detection, treatment and/or monitoring of renal disorders (e.g. Alport Syndrome and Polycystic Kidney Disease) by detecting genetic variations in relevant genes such as COL4A5, PKD1 and PKD2.

In some embodiments, the systems and methods described herein can be used can be used in the detection, treatment and/or monitoring of adrenal disorders (e.g. Congenital Adrenal Hyperplasia) by detecting genetic variations in relevant genes such as CYP21A2.

In some embodiments, the systems and methods described herein can be used can be used in the detection, treatment and/or monitoring of neurodevelopmental disorders (e.g. Rett Syndrome) by detecting genetic variations in relevant genes such as FOXG1, and MeCP2.

In some embodiments, the systems and methods described herein can be used can be used in the detection, treatment and/or monitoring of cerebrovascular disorders (e.g. Cerebral Cavernous Malformations) by detecting genetic variations in relevant genes such as KRIT1 and PDCD10.

In some embodiments, the systems and methods described herein can be used can be used in the detection, treatment and/or monitoring of neuro-oncology (e.g. Neurofibromatosis Type 1 and Neurofibromatosis Type 2) by detecting genetic variations in relevant genes such as NF1 and NF2.

In some embodiments, the systems and methods described herein can be used can be used in the detection, treatment and/or monitoring of epilepsy (e.g. Unverricht-Lundborg disease) by detecting genetic variations in relevant genes such as CSTB.

In some embodiments, the systems and methods described herein can be used can be used in the detection, treatment and/or monitoring of peripheral neuropathy by detecting genetic variations in relevant genes such as GJB1 and PMP22.

Use systems and methods described herein with existing clinical sequencing methods. In some cases, a sample can be analyzed using short-read sequencing to detect SNVs and indels, and long-read sequencing to detect SVs.

### Kits

In some embodiment, a kit Is described herein. The kit may comprise a plurality of crRNA probes disclosed herein. Further, the kit may comprise a plurality of tracerRNA molecules. The kit may comprise reagents that can be used to performing dA tailing and adapter ligation. Moreover, the kit may comprise any buffer that can be used in performing needed experiments. The kit may comprise instructions for performing any experiments and procedures described herein.

### Computer Systems

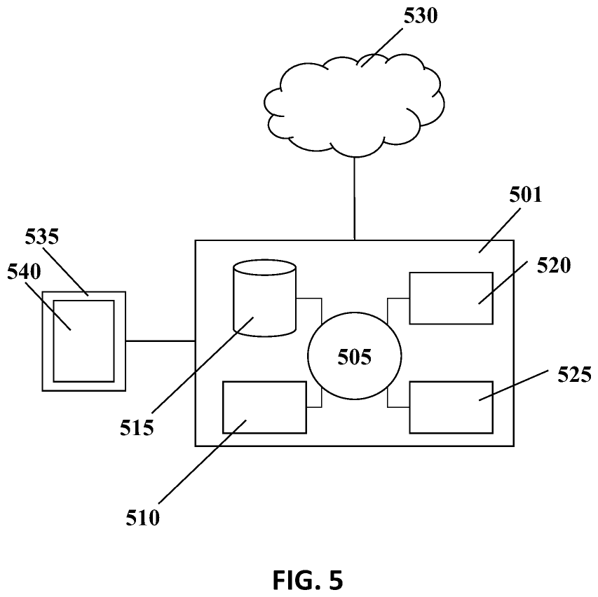

The present disclosure provides computer systems that are programmed to implement methods of the disclosure. FIG. 5 shows an example computer system 501 that can be programmed or otherwise configured to, for example, process and/or analyze a metabolite, control addition of reagents to reaction mixtures, control partition generation, control of reagent addition to partitions, provide conditions sufficient to conduct reactions, obtain and process sequencing data, output sequencing results to a user, provide an interface for user input to control devices coupled to the computer processor. The computer system 501 can regulate various aspects of the present disclosure, such as, for example, regulating fluid flow, delivery of reagents, partition generation, modulate reactions conditions, etc. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, monitoring of sample preparation, monitoring of reactions and/or reaction conditions, monitoring of sequencing, results of sequencing, and permitting user inputs for sample preparation, reactions, sequencing and/or sequencing analysis. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, implement sample preparation protocols, reaction protocols, sequencing protocols, data analysis protocols and system or device operation protocols.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a cell. For example, a biological particle or analyte carrier (e.g., a cell or cell bead) is partitioned in a partition (e.g., droplet), and multiple analytes from the biological particle or analyte carrier are processed for subsequent processing. The multiple analytes may be from the cell. This may enable, for example, simultaneous proteomic, transcriptomic and genomic analysis of the cell.

### EXAMPLES

### Example 1: Target Enrichment Protocol

An exemplary target enrichment protocol begins with preparing the Cas9 ribonucleoprotein complexes (RNPs). Prior to guide RNA assembly, all crRNAs are pooled into an equimolar mix, with a total concentration of 50-100 μM. The crRNA mix and tracrRNA are then combined such that the tracrRNA concentration and the total crRNA concentration are both 5-10 μM. The gRNA duplexes are formed by denaturation at 95° C. and then cooling to room temperature. Ribonucleoprotein complexes (RNPs) are constructed by combining the gRNA duplexes with Cas9 nucleases and then incubating at room temperature.

The next stage comprises dephosphorylating the genomic DNA. Between one to four genomic DNA samples can be pooled into the dephosphorylation reaction, for a total of 1-5 μg of gDNA in each phosphorylation reaction. The input DNA is dephosphorylated using Calf Intestinal Phosphatase or Shrimp Alkaline Phosphatase.

The next stage comprises cleaving and dA-tailing target DNA. RNPs are added to the dephosphorylated gDNA along with dATP and Taq DNA polymerase. The sample is then incubated at 37° C. for Cas9 cleavage followed by 72° C. for dA-tailing. The reaction is then cleaned up using SPRI beads. Next is barcode ligation. Barcodes are ligated to the dA-tailed ends of the gDNA using ligase. The reaction is incubated at room temperature and then cleaned up using SPRI beads.

Next stage is sequencing adapter ligation and clean-up. All the barcoded DNA are pooled together at an equimolar amount. Sequencing adapters are ligated to the pool of barcoded DNA using ligase. The DNA is then cleaned up using SPRI beads, and then eluted in elution buffer.

The next stage is priming and loading the Flow Cell. Libraries were prepared for sequencing by adding the following to the eluate: Sequencing Buffer, Loading Beads, and Flush Tether. The sequencing libraries are then loaded onto the flow cell for sequencing.

### Example 2: BRCA1 crRNA Probe Design

In the case of BRCA1, the CHOPCHOP design program yielded a total of 5567 possible crRNA probes along the entire length of the BRCA1 genomic locus. These crRNA sequences were then filtered using the filtering scheme described in [0041], reducing the number to 233 crRNA probes. The crRNA sequences were then checked using a second design checker tool, e.g. IDT CRISPR-Cas9 guide RNA design checker tool. The number of candidate crRNA probes was reduced to 86 probes. The final set of crRNA probes was chosen based upon the location of the target sites.

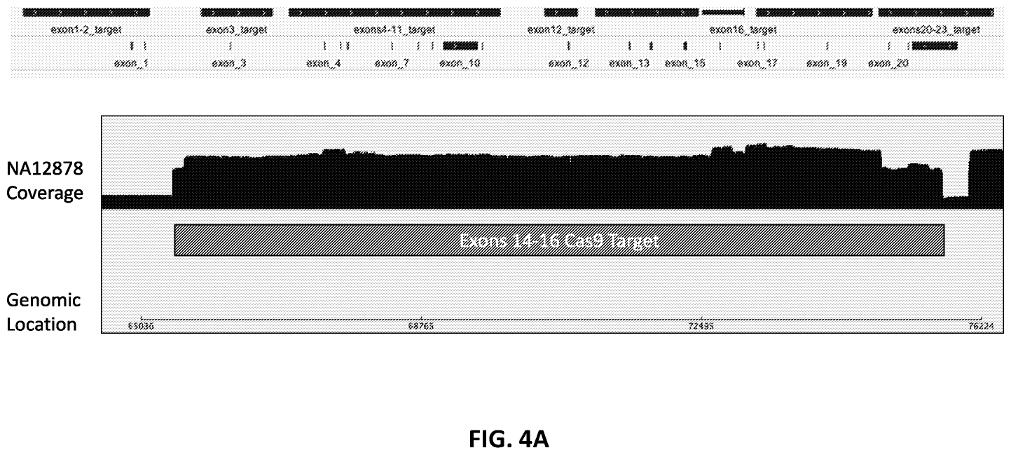

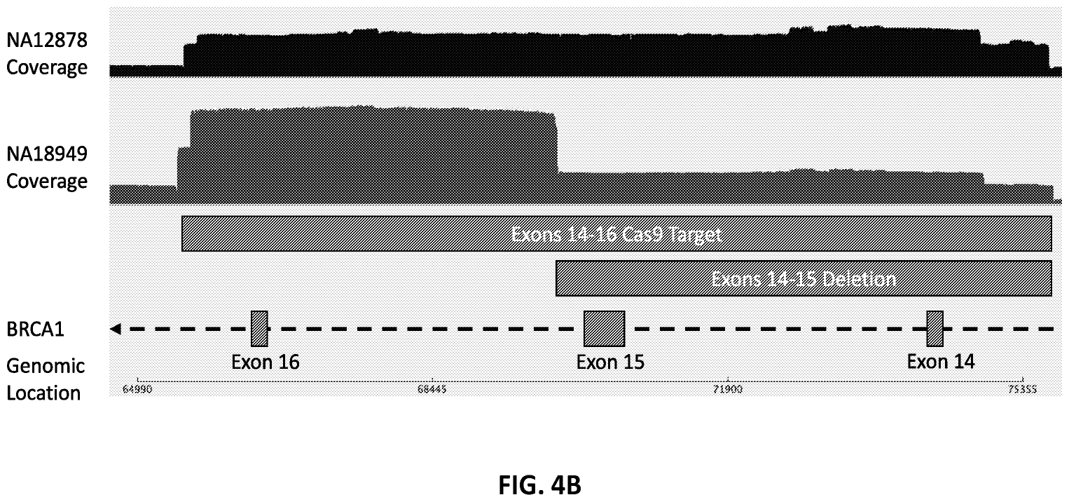

As shown in FIG. 4A, successful Cas9 cleavage and sequencing results in increased sequencing coverage of the target region with little or no sequencing coverage of non-target regions. In a sample with a known deletion of exons 15 and 16, a sharp drop in sequencing coverage is observed where the deletion occurs (FIG. 4B).

While preferred embodiments of the present invention have been shown and described herein, it will be obvious to those skilled in the art that such embodiments are provided by way of example only. It is not intended that the inventions be limited by the specific examples provided within the specification. While the invention has been described with reference to aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the inventions are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for identifying a set of guide RNAs (gRNAs) that are hybridizable to a genomic region of interest in a genome comprising designing a plurality of gRNAs, wherein:
at least one gRNA is hybridizable to a target site within the genomic region of interest and is configured to produce a genomic variant that comprises at least 1000 bp; and
said plurality of gRNAs comprises a plurality of CRISPR RNAs (crRNAs), wherein said plurality of crRNAs comprises a GC of at least about 40% to about 80%.

2. A method for identifying a set of guide RNAs (gRNAs) that are hybridizable to a genomic region of interest in a genome comprising designing a plurality of gRNAs, wherein:
at least one gRNA is hybridizable to a target site within the genomic region of interest and is configured to produce a genomic variant that comprises at least 1000 bp; and
said plurality of gRNAs comprises a plurality of CRISPR RNAs (crRNAs), wherein said plurality of crRNAs comprises a self-complementarity score of zero.

3. A method for identifying a set of guide RNAs (gRNAs) that are hybridizable to a genomic region of interest in a genome comprising designing a plurality of gRNAs, wherein:
at least one gRNA is hybridizable to a target site within the genomic region of interest and is configured to produce a genomic variant that comprises at least 1000 bp; and
said plurality of gRNAs comprises a plurality of CRISPR RNAs (crRNAs), wherein said plurality of crRNAs comprises an efficiency score of about 0.2.

4. A method for identifying a set of guide RNAs (gRNAs) that are hybridizable to a genomic region of interest in a genome comprising designing a plurality of gRNAs, wherein:
at least one gRNA is hybridizable to a target site within the genomic region of interest and is configured to produce a genomic variant that comprises at least 1000 bp; and
said plurality of gRNAs comprises a plurality of CRISPR RNAs (crRNAs), wherein said plurality of crRNAs comprises a mismatch profile of MM0=0 or MM0=1, MM1=0 or MM1=1, MM2=0 or MM2=2, and MM3<21.

5. The method of claim 4, wherein said plurality of crRNAs comprises a mismatch profile of MM3<5.

6. A method of detecting a genomic variant in a sample, the method comprising:
(a) enriching said sample for a genomic region of interest comprising said genomic variant using a gene-editing based approach; and
(b) sequencing said enriched sample comprising said genomic region of interest using long-read sequencing.

7. The method of any of claims 1-6, wherein said genomic variant comprises a structural variant.

8. The method of claim 5, wherein said genomic variant comprises at least 50 bp.

9. The method of claim 5, wherein said genomic variant comprises at least 1000 bp.

10. The method of any of claims 1-5, wherein said gene-editing based approach comprises use of a clustered regularly interspersed short palindromic repeats (CRISPR)-Cas system.

11. The method of claim 9, wherein said CRISPR-Cas system comprises Cas 9.

12. The method of claim 5, wherein step (a) of enriching said sample further comprises an amplification of said genomic region of interest.

13. The method of claim 5, wherein step (a) of enriching said sample does not require an amplification of said genomic region of interest.

14. The method of claim 5, wherein step (a) of enriching said sample further comprises coupling a sequence of dAMPs to said genomic variant.

15. The method of claim 5, wherein step (a) of enriching said sample further comprises coupling a plurality of barcode molecules to said genomic variant.

16. The method of claim 5, wherein step (a) of enriching said sample further comprises coupling said genomic variant to a magnetic bead.

17. The method of any of claims 1-5, wherein said long-read sequencing comprises nanopore sequencing.

18. The method of any of claims 1-5, wherein said long-read sequencing comprises single molecule, real-time (SMRT) sequencing.

19. The method of any of claims 1-5, wherein said CRISPR-Cas system further comprises a crRNA that is hybridizable to a sequence listed in Tables 1-117.

20. The method of any of claims 1-5, wherein said genomic region of interest comprises two or more repeat regions.

21. The method of any of claims 1-5, wherein said sample comprises at least 10 genomic regions of interest.

22. The method of any of claims 1-5, wherein said genomic variant is associated with a disorder and drug response (pharmacogenomics).

23. The method of claim 22, wherein said disorder is selected from the group consisting of acute lymphoblastic leukemia (ALL), alpha-thalassemia, ataxia-telangiectasia (AT), autosomal recessive deafness 16, autosomal recessive deafness 22, beta-thalassemia, breast cancer, Canavan disease, cancer, celiac disease, chronic myeloid leukemia (CML), cystic fibrosis, cystinosis, deafness infertility syndrome (DIS), Duchenne muscular dystrophy, Ehlers-Danlos syndrome type III and IV, Ellis-van Creveld syndrome, Fabry disease, familial adenomatous polyposis (FAP), familiar cutaneous melanoma, Fragile X, gastric cancer (including hereditary diffuse gastric cancer), Gaucher disease, hereditary predisposition to develop cancer, Huntington disease, hypophosphatasia (HPP), incontinentia pigmenti, Krabbe disease, Leber congenital amaurosis (LCA), Loeys-Dietz syndrome, Long QT syndrome, Lynch syndrome, Marfan syndrome, mental disorder, medium-chain acyl-coenzyme A dehydrogenase (MCAD) deficiency, MUTYH-associated polyposis, neuroblastoma, neuronal ceroid-lipofuscinoses (NCLs), Niemann-Pick Type C disease, pancreatic cancer syndromes, papillary renal carcinoma, Parkinson disease, phenylketonuria, Pompe disease, propiopnic acidemia, rheumatoid arthritis, solid tumors, spinal muscular atrophy, spinocerebellar ataxia, susceptibility to breast cancer, Tay-Sachs disease, very long-chain acyl-coenzyme A dehydrogenase deficiency, Von Hippel-Lindau syndrome, Wilms tumor, Wilson disease, Wolfram syndrome type 1, X-linked creatine deficiency syndrome, X-linked hemophilia A, and X-linked retinitis pigmentosa.

24. A method of designing a probe to target a genomic region of interest, the method comprising:
(c) designing a plurality of nucleic acid probe options to target said genomic region of interest;
(d) selecting a first set of candidates from said plurality of nucleic acid probe options with a GC content of at least 20%;
(e) selecting a second set of candidates from said first set of candidates with a self-complementarity score of zero or a complementarity score of 1;
(f) selecting a third set of candidates from said second set of candidates with an efficiency greater than or equal to 0.2; and
(g) selecting a fourth set of candidates from said third set of candidates with a mismatch profile of MM0=0 or MM0=1, MM1=0 or MM1=1 or MM1=2, MM2=0 or MM2=1 or MM2=2, and MM3<21, wherein said fourth set of candidates comprises said probe to target a genomic region of interest.

25. The method of claim 24, wherein fourth set of candidates comprises a mismatch profile of MM3<5.

26. The method of claim 24, wherein said designing comprises using CHOPCHOP.

27. The method of claim 24, wherein said first set of candidates have a GC content of about 40% to about 80%.

28. The method of claim 24, wherein said nucleic acid probe of interest comprises a crRNA.

29. The method of claim 27, wherein a probability of said crRNA cutting said genomic region of interest is greater than or equal to 80%.

30. The method of claim 21, further comprising estimating on-target value of said crRNA.

31. The method of claim 29, further comprising estimating off-target value of said crRNA.

32. A kit comprising a set of guide RNAs (gRNAs) that are hybridizable to a genomic region of interest in a genome comprising designing a plurality of gRNAs, wherein:
at least one gRNA is hybridizable to a target site within the genomic region of interest and is configured to produce a genomic variant that comprises at least 1000 bp; and
said plurality of gRNAs comprises a plurality of CRISPR RNAs (crRNAs), wherein said plurality of crRNAs comprises a GC of at least about 40% to about 80%.

33. A kit comprising a set of guide RNAs (gRNAs) that are hybridizable to a genomic region of interest in a genome comprising designing a plurality of gRNAs, wherein:
at least one gRNA is hybridizable to a target site within the genomic region of interest and is configured to produce a genomic variant that comprises at least 1000 bp; and
said plurality of gRNAs comprises a plurality of CRISPR RNAs (crRNAs), wherein said plurality of crRNAs comprises a self-complementarity score of zero.

34. A kit comprising a set of guide RNAs (gRNAs) that are hybridizable to a genomic region of interest in a genome comprising designing a plurality of gRNAs, wherein:
at least one gRNA is hybridizable to a target site within the genomic region of interest and is configured to produce a genomic variant that comprises at least 1000 bp; and
said plurality of gRNAs comprises a plurality of CRISPR RNAs (crRNAs), wherein said plurality of crRNAs comprises an efficiency score of about 0.2.

35. A kit comprising a set of guide RNAs (gRNAs) that are hybridizable to a genomic region of interest in a genome comprising designing a plurality of gRNAs, wherein:
at least one gRNA is hybridizable to a target site within the genomic region of interest and is configured to produce a genomic variant that comprises at least 1000 bp; and
said plurality of gRNAs comprises a plurality of CRISPR RNAs (crRNAs), wherein said plurality of crRNAs comprises a mismatch profile of MM0=0 or MM0=1, MM1=0 or MM1=1, MM2=0 or MM2=2, and MM3<21.
